(19) United States
(12) Patent Application Publication
(10) Pub. No.: US 2025/0110117 A1
(43) Pub. Date: Apr. 3, 2025

(54) SYSTEMS OF SINGLE-CELL LABELING AND METHODS OF USING THE SAME

(71) Applicant: The Wistar Institute of Anatomy and Biology, Philadelphia, PA (US)

(72) Inventor: Amelia ESCOLANO, Philadelphia, PA (US)

(73) Assignee: The Wistar Institute of Anatomy and Biology, Philadelphia, PA (US)

(21) Appl. No.: 18/902,306

(22) Filed: Sep. 30, 2024

Related U.S. Application Data
(60) Provisional application No. 63/586,215, filed on Sep. 28, 2023.

(51) Int. Cl. G01N 33/50; C12N 9/12; C12N 15/86; C12Q 1/6869; G01N 33/68

(52) U.S. Cl. CPC G01N 33/5052; C12N 9/1241; C12N 15/86; C12Q 1/6869; G01N 33/6818; C12N 2740/16122; C12N 2750/14122; C12N 2750/14143

(57) ABSTRACT

The disclosure provides materials and methods for permanently labeling cells by transducing cells with viruses comprising a barcode. The disclosure is based on the idea that tracking, selecting and isolating lymphocytes labeled with the barcode can facilitate identification of clonal cells responsible for antibody or antibody fragment production.

### CROSS-REFERENCE TO RELATED APPLICATIONS

This application claims the benefit of U.S. Provisional Application No. 63/586,215, filed on Sep. 28, 2023, the contents of which are hereby incorporated by reference in its entirety.

### STATEMENT REGARDING FEDERALLY SPONSORED RESEARCH

This disclosure was made with government support under grant number AI175470 awarded by the National Institutes of Health. The government has certain rights in this disclosure.

### SEQUENCE LISTING

The instant application contains a Sequence Listing which has been submitted electronically in XML format and is hereby incorporated by reference in its entirety. Said XML copy, created on Sep. 30, 2024, is named WIST-012_SL.xml and is 32,760 bytes in size.

### FIELD

The disclosure relates to compositions comprising cells comprising an exogenous DNA label and endogenous nucleic acid sequences that encode immune-associated molecules. In some embodiments, the composition comprises cells such as B cells that comprise an exogenous nucleic acid sequence barcode integrated within in the cell's endogenous DNA, and methods of making the same using gene-editing enzymes.

### BACKGROUND

Infectious diseases are serious and recurrent health threats. Efficacious vaccines to prevent infection by current or newly arising pathogens are highly desirable. Particularly concerning are viruses with the capacity to rapidly mutate. HIV-1, influenza or SARS-CoV2 have the ability to mutate to adapt to new hosts and environments, escaping from the pressure exerted by the host immune system. This ability to mutate results in a large diversity of circulating viral variants, characterized by individual antigenic and infectivity properties. Efficacious vaccines to broadly protect against all these variants should induce antibodies against their conserved viral epitopes; however, directing antibody responses to conserved epitopes by vaccination is incredibly challenging. Failure to do so results in polyclonal antibody responses to different non-conserved viral epitopes, which severely interfere with the development of broadly protective responses. Antibodies mature in the germinal centers, the anatomical sites where B cells proliferate upon encountering cognate antigen, and their B cell receptors (BCRs) undergo somatic hypermutation and affinity-based selection in competition with multiple other B cell clones. Interestingly, previous studies demonstrated that repeated immunization with the same priming immunogen did not induce antibody maturation; Instead, a novel form of vaccination, involving sequential immunization with a series of different engineered and native-looking Env-based immunogens, was required to produce antibodies that could bind and neutralize the native HIV-1 (FIG. 1A). Sequential immunization sustained multiple rounds of somatic hypermutation and selection in the germinal centers, which resulted in matured, highly mutated antibodies. These immunization experiments were performed in a pioneer immunoglobulin knock-in (Ig KI) mouse model, which was produced to carry a monoclonal B cell repertoire. All B cells in this mouse expressed the same antibody, the unmutated precursor of a human anti HIV-1 bNAb. In this scenario, there was no competition between different B cell clonotypes and immunodominance played no role (FIG. 1B). These studies were proof of concept that anti-HIV-1 bNAbs can be elicited by vaccination, but antibody maturity and selection processes are needed to ensure therapeutically effective compositions capable of inhibiting virus function. Selection of B cells that are permanently labeled by barcodes do not exist and there are limited techniques available to create or manipulate B cells to identify, isolate and assay mature antibodies.

### SUMMARY OF EMBODIMENTS

The disclosure relates to a composition comprising a cell or plurality of cells labeled with one or a plurality of exogenous barcodes, each cell labeled with an independently selected or independently selectable exogenous barcode. In some embodiments, such barcode being configured for expression with, in cis with, or sequentially with expression of an immunologic molecule, such as an antibody or antibody fragment. In some embodiments, the cell is an antigen-presenting cell. In some embodiments, the cell is a B cell. In some embodiments, the cell is a specialized or differentiated B cell, such as a memory B cell or plasma cell.

The disclosure relates to methods of labeling a cell with an endogenous barcode or endogenously expressed label comprising: (a) exposing one or a plurality of cells to an attenuated viral particle comprising a nucleic acid molecule, the nucleic acid molecule comprising an exogenous nucleic acid sequence comprising a barcode and a nucleic acid sequence encoding a gene-editing enzyme, wherein the gene editing enzyme is configured to associate with and/or edit an endogenous target DNA sequence within the cell. In some embodiments, the step of exposing is performed for a time period sufficient to allow expression of the gene-editing enzyme and editing of the endogenous DNA sequence of the cell, such that the endogenous DNA of the cell is modified to include a region of exogenous barcode DNA sequence from the nucleic acid molecule.

The disclosure also relates to methods of labeling a cell with an endogenous barcode or endogenously expressed label comprising: (a) exposing one or a plurality of cells to a viral particle comprising a chimeric amino acid sequence, wherein the chimeric amino acid sequence comprises a first amino acid domain and a second amino acid domain; wherein the first amino acid domain mediates binding of an antigen to the viral particle; and a second amino acid domain comprises an antigen. In some embodiments, the method further comprises a step of exposing the cell to a nucleic acid molecule comprising a first nucleic acid sequence comprising a barcode and a second nucleic acid sequence that encodes a gene-editing enzyme recognition site. In some embodiments, the method further comprises a step of exposing the cell to a gene-editing enzyme, or alternatively, a nucleic acid encoding a gene-editing enzyme, for a time period sufficient to allow the gene-editing enzyme to modify an endogenous DNA target of the cell with addition of the barcode. In some embodiments, the viral particle comprises one or more AAV capsid proteins and the viral particle displays SpyCatcher or a functional variant thereof. In some embodiments, the viral particle is an AAV particle that comprises SpyCatcher, a functional variant thereof, and a ligand for SpyCatcher. In some embodiments, the ligand for SpyCatcher is a chimeric protein with a first domain and a second domain; wherein the first domain is a SpyTag configured to bind or associate with SpyCatcher, and wherein the second region is an antigen. In some embodiments, the antigen comprises a tumor associated antigen, a cancer antigen or a pathogen antigen. In some embodiments, the pathogen antigen is a viral antigen. In some embodiments, the viral particle is an attenuated virus or non-pathogenic virus.

In some embodiments, the method is performed in vitro or in vivo. If the methods are performed in vivo, in some embodiments, a step of administering to a subject one or a plurality of viral particles or composition comprising viral particles herein is performed prior to the step of exposing. In some embodiments, the step of administering one or a plurality of viral particles is performed by oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intraperitoneal administration, intramuscular administration, and subcutaneous administration. Administration can be continuous or intermittent. In some embodiments, the gene editing enzyme is a meganuclease, transposase or Cas protein. In some embodiments, the gene editing enzyme is a SleepingBeauty transposase or variant thereof that is highly active as compared to the wild type SleepingBeauty amino acid sequence.

The disclosure further provides a method of tracking or identifying an antigen-specific B cell after an exposure to one or a plurality of antigens. Tracking antigen-specific B cells will illuminate the mechanisms of epitope immunodominance, memory B cell reactivation and B cell fate into the memory and plasma cell compartments. In some embodiments, methods of the disclosure include a method of identifying and/or isolating a B cell comprising: (a) exposing a subject to an antigen for a time period and under conditions required to elicit an antigen-specific immune response; and (b) exposing one or a plurality of cells to a viral particle comprising a nucleic acid molecule, the nucleic acid molecule comprising an exogenous nucleic acid sequence comprising a barcode and a nucleic acid sequence encoding a gene-editing enzyme, wherein the gene editing enzyme is configured to associate with and/or edit an endogenous DNA sequence of the cell specific for expression of an immune molecule; and wherein the step of exposing is for a time period sufficient to allow expression of the gene-editing enzyme and to allow editing of the endogenous DNA sequence of the cell, such that the endogenous DNA of the cell is modified to comprise a region of exogenous DNA sequence from the nucleic acid molecule. In some embodiments, the methods also include administering one or a plurality of pharmaceutical compositions comprising a vaccine, wherein the vaccine comprises an antigen or a nucleic acid sequence encoding an antigen before exposure to the viral particle. In some embodiments, the vaccine is exposed to a cell in vivo or in vitro for a time period sufficient to elicit an antigen-immune response against the antigen. In some embodiments, the method of isolating or identifying further comprises a step of isolating a sample from the subject after administration of the vaccine and/or exposure of the subject to the composition comprising the viral particle. In some embodiments, peripheral blood monocytes or B cells are isolated from the sample by flow cytometry after centrifuging a sample. In some embodiments, the methods further comprise isolating an activated B cell after administration of a vaccine or exposure to an antigen by taking a blood sample from the subject, isolating lymphocytes from a blood sample by centrifugation, flow cytometry, ion exchange chromatography, immunohistochemistry and/or immune precipitation. In some embodiments, methods of the disclosure include further steps of (i) analyzing the cells for production of immunoreactive antibody or antibody fragments raised against the antigen to which the cell was exposed; (ii) correlating the isolated cell with the epitope corresponding to a fragment of the antigen against which the cell was exposed; and (iii) identifying a DNA barcode sequence associated with the epitope or the immunoreactive antibody or antibody fragment, such that a DNA barcode sequence corresponds to a reactive B cell. In some embodiments, the step of identifying a DNA barcode comprises sequencing the barcode, and, in some embodiments, by polymerase chain reaction.

This disclosure also provides a method of permanently tagging antigen-recognizing immune cells upon sequential immunization, that will provide the ability to identify, classify and track these cells either in vitro or in vivo. The method comprises exposing a disclosed viral particle comprising a nucleic acid molecule comprising one or a plurality of DNA barcodes and a nucleic acid sequence encoding a gene-editing enzyme, such as a transposase, meganuclease, or a Cas protein, wherein, if the gene-editing enzyme is a Cas protein, the viral particle optionally further comprises an sgRNA molecule specific for association with the Cas protein and a sequence partially complementary to the endogenous DNA of the cell or a DNA sequence encoding an sgRNA molecule specific for association with the Cas protein and the DNA barcode and a sequence at least partially complementary to the endogenous DNA of the cell. In some embodiments, the method further comprises allowing the viral particle to infect the cell and modify the endogenous DNA of the cell, such that the DNA barcode is stably integrated within the endogenous DNA of the cell. In some embodiments, the method further comprises exposing the cell to antigen to elicit an antigen-specific immune response before or after the steps of exposing or allowing.

This disclosure also provides a method of tracking or identifying an immune cell comprising exposing a disclosed viral particle comprising a nucleic acid molecule comprising one or a plurality of DNA barcodes and a nucleic acid sequence encoding a gene-editing enzyme, such as a transposase, meganuclease, or a Cas protein, wherein, if the gene-editing enzyme is a Cas protein, the viral particle optionally further comprises an sgRNA molecule specific for association with the Cas protein and a sequence partially complementary to the endogenous DNA of the cell or a DNA sequence encoding an sgRNA molecule specific for association with the Cas protein and the DNA barcode and a sequence at least partially complementary to the endogenous DNA of the cell. In some embodiments, the method further comprises allowing the viral particle to infect the cell and modify the endogenous DNA of the cell, such that the DNA barcode is stably integrated within the endogenous DNA of the cell and the cell is labeled with the DNA barcode. In some embodiments, the method further comprises exposing the cell to an antigen to elicit an antigen-specific immune response before or after the steps of exposing or allowing. In some embodiments, the method further comprises identifying the immune cell comprising the stably integrated DNA barcode by isolating immune cells from a subject or immune cells from a population of other cells in vitro, sequencing the DNA of the cells, detecting the sequence of the DNA barcode, such that identification of the sequence of the DNA barcode is indicative of identifying the labeled cell. In some embodiments, the method further comprises correlating the identification of the labeled cell with an expression profile. In some embodiments, the expression profile comprises expression of an antibody specific to the antigen and specifically secreted by the labeled cell.

The hypothesis is that tracking antigen-specific B cells upon sequential immunization, will guide the design of efficacious vaccines against highly mutating pathogens. Tracking antigen specific B cells responding to sequential immunization will illuminate the mechanisms of epitope immunodominance, memory B cell reactivation and B cell fate into the memory and plasma cell compartments.

In some aspects, the disclosure provides a novel nucleic acid molecule, wherein the nucleic acid molecule comprises an engineered AAV genome comprising:
(i) a barcode DNA;
(ii) a nucleic acid sequence encoding a gene editing enzyme or functional variant thereof;
wherein the nucleic acid sequence encoding the gene editing enzyme or functional variant thereof is operably linked to a regulatory sequence; and
(iii) a first gene editing enzyme cleavage sequence and a second gene editing enzyme cleavage sequence; wherein the first gene editing enzyme cleavage sequence is positioned within about 20 nucleotides upstream from the 5′ end of barcode; and wherein the second gene editing enzyme cleavage sequence is positioned within about 20 nucleotides downstream from the 3′ end of the barcode DNA.

In some embodiments, the nucleic acid sequence encoding a gene editing enzyme or functional variant thereof encodes a transposase, meganuclease, a Cas protein or a functional variant thereof; wherein a first gene editing enzyme cleavage sequence and a second gene editing enzyme cleavage sequence is a transposase-, meganuclease-, or Cas protein recognition and cleavage site, respectively. In some embodiments, the nucleic acid sequence encoding a gene editing enzyme or functional variant thereof encodes a transposase or functional variant thereof. Generally, in some embodiments and in 5′ to 3′ orientation, the engineered AAV genome comprises:
(i) a nucleic acid sequence encoding a gene editing enzyme or functional variant thereof;
(ii) a first gene editing enzyme cleavage sequence;
(iii) a barcode sequence; and
(iv) a second gene editing enzyme cleavage sequence.

In some embodiments, the nucleic acid molecule comprises a first and second inverted tandem repeat sequence positioned at or proximate to the 5′ end and 3′ end of the nucleic acid molecule, respectively. In some embodiments, the first and second inverted tandem repeat (ITR) sequences comprise AAV ITR sequences.

In some embodiments, the disclosure provides a liposome, a cell or a virus comprising the nucleic acid molecule, wherein the disclosed nucleic acid molecule is positioned within the liposome, cell or virus.

In some embodiments, the virus is an AAV pseudovirus. In some embodiments, the AAV pseudovirus is replication-deficient.

In some embodiments, the disclosure relates to a cell comprising a barcode DNA within its genome comprising the AAV, ITR, at least one DNA barcode sequence; wherein the DNA barcode is detectable by PCR, qPCR and/or NGS. In some embodiments, the cell further comprises either (i) a second vector expressing AAV capsid proteins and a third vector expressing AAV helper proteins, or (ii) a second vector expressing AAV capsid proteins and AAV helper proteins. In some embodiments, the cell further comprises an antigen, or one or more epitopes from the antigen which is fused to a ligand, the ligand specific for a ligand binding protein or peptide. In some embodiments, in a population of cells, one cell comprises an antigen or one or more epitopes from an antigen and another cell comprises a different barcode, such that each cell or population of cells is differentially barcoded and each cell or population of cells expresses a different antigen or one or more different epitope of the antigen and each barcode can be paired with the antigen or epitope of the antigen to which the cell is exposed.

In some embodiments, the disclosure provides a vaccine or a composition comprising a viral particle disclosed herein comprising a nucleic acid molecule comprising: (i) a DNA barcode; and (ii) at least two gene-editing enzyme recognition sequences. In some embodiments, the composition further comprises a nucleic acid sequence encoding a gene-editing enzyme, such as a transposase. In some embodiments, the composition further comprises one or a plurality of regulatory sequence operably linked to the nucleic acid encoding a gene-editing enzyme.

In some embodiments, the disclosure provides a kit comprising:
(i) the nucleic acid molecule of the disclosure; or
(ii) a viral vector comprising the nucleic acid molecule of the disclosure; or
(iii) a plurality of viral vectors, wherein at least one viral vector comprises a nucleic acid sequence encoding a gene-editing enzyme and at least one viral vector comprises a nucleic acid molecule comprising a DNA barcode positioned between a 5′ and a 3′ viral packaging sequence. In some embodiments, the Kit comprises a plurality of pseudotyped viral particles, comprising an AAV capsid protein and a DNA barcode, wherein the DNA barcode of a first viral particle or first set of vial particles is different than a second viral particle or second set of viral particles. In some embodiments, the kit comprises a plurality of viral particles, each viral particle comprising a nucleic acid molecule comprising a different DNA barcode. In some embodiments, the DNA barcode is a DNA sequence comprising a random sequence and in other embodiments, the DNA barcode comprises a known sequence.

In some embodiments, the kit further comprises (iii) a helper plasmid. In some embodiments the kit further comprises (iv) a transfection buffer.

The disclosure also provides a method for manufacturing a viral particle comprising transfecting cells with a transfection mixture comprising the nucleic acid molecule disclosed herein, wherein at least one nucleic acid molecule comprises a nucleic acid sequence encoding an antigen or antigen fragment thereof fused to a ligand that specifically binds to a ligand-binding protein or peptide on the viral particle. After transfection with the nucleic acid molecule of the disclosure and a help plasmid, in some embodiments, the transfected cells produce viral particles that comprise a series of different barcodes and a gene-editing recognition sequence, such that upon exposure to a target cell, such as a B cell, the target cell genome is modified to include the DNA barcode.

Methods of the disclosure also include methods of labeling an immune cell by exposing the immune cell to a viral particle. In some embodiments, the viral particle is a non-pathogenic viral particle, such as AAV particle that comprises a modified genome sequence. The modification in the genome ensures that the virus is non-replicating and that it carries components that allow for gene editing in the presence of a gene-editing enzyme.

In some aspects, the disclosure provides a method for labeling an immune cell with a barcode comprising:
(a) exposing a population of immune cells to a transfection mixture comprising the composition according to the disclosure and a non-toxic carrier or diluent for a time period sufficient for the virus particle comprising the nucleic acid molecule comprising a barcode to enter the cells; and
(b) exposing the nucleic acid molecule comprising the barcode to a gene editing enzyme for a time period sufficient for the gene editing enzyme to excise the nucleic acid sequence comprising the barcode from the nucleic acid molecule; and integrate the barcode into genomic DNA of the cells. In some embodiments, the method further comprises culturing an antigen reactive cell for a time period sufficient for clonal expansion of the cell. In some embodiments, the immune cell is a B cell.

In some aspects, the disclosure provides a method for identifying antigen-specific cells comprising the method comprising: (a) exposing a population of immune cells to a transfection mixture comprising the composition according to the disclosure and a non-toxic carrier or diluent for a time period sufficient for the virus particle comprising the nucleic acid molecule comprising a barcode to enter the cells; and
(b) exposing the nucleic acid molecule comprising the barcode to a gene editing enzyme for a time period sufficient for the gene editing enzyme to excise the nucleic acid sequence comprising the barcode from the nucleic acid molecule; and integrate the barcode into genomic DNA of the cells. In some embodiments, the method further comprises isolating the cell from a culture of cells after steps (a) and (b), and sequencing the barcode. In some embodiments, the method further comprising sequencing one or more immunoreactive proteins expressed in the cell and profiling the cell as expressing the immunoreactive protein and carrying the barcode. In some embodiments, the cell is a B cell and the method further comprises a step of correlating and/or cataloguing the barcode to the cell and the immunoreactive protein being expressed by the cell. In some embodiments, the immunoreactive protein being expressed by the cell is an antibody or antibody fragment stimulated in response to the B cell being exposed to an antigen prior to, simultaneous with or subsequent to exposure to the viral particle.

The disclosure also provides a method for determining epitope specificity of B cells comprising the method comprising: (a) exposing a population of B cells to the composition according to the disclosure comprising a non-toxic carrier or diluent for a time period sufficient for the virus particle comprising the nucleic acid molecule comprising a barcode to enter the cells; and
(b) exposing the nucleic acid molecule comprising the barcode to a gene editing enzyme, or a nucleic acid sequence encoding the gene editing enzyme, for a time period sufficient for the gene editing enzyme to excise the nucleic acid sequence comprising the barcode from the nucleic acid molecule and integrate the barcode into genomic DNA of the B cells. In some embodiments, the method further comprises exposing the B cell to an antigen or fragment thereof and allowing for a time period for the B cell to respond to the antigen or fragment thereof by expressing an antigen-specific immunoreactive protein corresponding to the antigen or fragment thereof. In some embodiments, the method further comprises determining epitope specificity by sequencing the immunoreactive protein. In some embodiments, the method further comprises isolating the cell from a culture of cells after steps above, and sequencing the barcode to correlate the barcode with the B cell expressing the immunoreactive protein or proteins. In some embodiments, the method further comprises sequencing the barcode only after allowing a time period to elapse after exposure of the B cell to the antigen or fragment thereof.

The disclosure also provides a method to produce one or a plurality of immunoreactive proteins reactive against an antigen comprising: (a) exposing a population of immune cells to a composition comprising: (i) a viral particle disclosed herein comprising a nucleic acid molecule comprising a barcode; and (ii) a non-toxic carrier or diluent for a time period sufficient for the virus particle comprising the nucleic acid molecule comprising a barcode to infect the immune cells; and
(b) exposing the nucleic acid molecule comprising the barcode to a gene editing enzyme for a time period sufficient for the gene editing enzyme to excise the nucleic acid sequence comprising the barcode from the nucleic acid molecule; and integrate the barcode into genomic DNA of the cells. In some embodiments, the method further comprises exposing the immune cell to an antigen either before, during or after exposure to the nucleic acid molecule. In some embodiments, the viral particle comprises the nucleic acid molecule comprising the barcode and the viral particle also contemporaneously exposes the immune cells to an antigen due to the viral particle comprising an antigen or fragment thereof. In some embodiments, the viral particle comprises a chimeric protein on its surface, the chimeric protein comprising a first and second protein domain, wherein the first protein domain comprises an antigen or antigen fragment, and the second protein domain comprises a ligand binding domain that associates with a ligand displayed on the capsid of the viral particle surface. In some embodiments, the method further comprises isolating the cell from a culture of cells after the steps of exposing, and sequencing the barcode to correlate the barcode sequence with the immunoreactive protein the immune cell expresses in response to exposure to the antigen.

The disclosure further provides a method for determining immunodominant epitopes in a subject comprising immunizing the subject with a plurality of viral particles comprising a plurality of antigens, the plurality of viral particles further comprising a plurality of DNA barcodes, exposing the subject to a having an antibody repertoire with a mixture of differentially barcoded vaccines and determining by barcode sequencing which antigens induce preferential differentiation toward memory B cells and which antigens induce preferential differentiation toward plasma cells.

The disclosure also provides a method for determining B cell recall comprising: exposing the B cell to an antigen, labeling the B cell with a barcode by exposing the B cell to a viral particle comprising a nucleic acid molecule with the barcode, and subsequently exposing the B cell to one or a plurality of compositions comprising the antigen, and measuring the antigen-specific immune response to the antigen to determine if the B cell developed recall. In some embodiments, the method further relates to sequencing the barcode integrated within the B cell and pairing the barcode sequence to the antigen-specific immune response. In some embodiments, the steps are repeated and catalogued such that an antigen-specific exposure history can be generated.

The disclosure provides a method for identifying epitope dominance of an antigen comprising immunizing a mammal having an antigen-specific antibody repertoire with a mixture of differentially barcoded vaccines; measuring the antigen-specific immune response of the antigen or across multiple antigens; and determining by barcode sequencing which B cells epitopes induce the greatest immune response to the antigen.

In some embodiments, the disclosure provides a method for identifying and characterizing B cell clones based upon the sites and/or presence of barcode integration by the method comprising: (a) exposing a population of B cells to a composition comprising: (i) a viral particle disclosed herein comprising a nucleic acid molecule comprising a barcode; and (ii) a non-toxic carrier or diluent for a time period sufficient for the virus particle comprising the nucleic acid molecule comprising a barcode to infect the immune cells; and
(b) exposing the nucleic acid molecule comprising the barcode to a gene editing enzyme for a time sufficient for the gene editing enzyme to excise the nucleic acid sequence comprising the barcode from the nucleic acid molecule and integrate the barcode into endogenous or genomic DNA of the cell. In some embodiments, the method further comprises performing inverse PCR (iPCR) or splinkerette PCR to determine the sequence of the barcode.

The disclosure relates to a nucleic acid molecule wherein the nucleic acid molecule comprises: (i) a barcode domain; (ii) a nucleic acid sequence encoding a gene editing enzyme or functional variant thereof; wherein the nucleic acid sequence encoding the gene editing enzyme or functional variant thereof is operably linked to a regulatory sequence; and (iii) a first gene editing enzyme cleavage sequence and a second gene editing enzyme cleavage sequence; wherein the first gene editing enzyme cleavage sequence is positioned within about 20 nucleotides upstream from the 5′ end of the nucleic acid sequence encoding a gene editing enzyme or functional variant thereof; and wherein the second gene editing enzyme cleavage sequence is positioned within about 20 nucleotides downstream from the 3′ end of the nucleic acid sequence encoding a gene editing enzyme or functional variant thereof. In some embodiments, the nucleic acid sequence encoding a gene editing enzyme or functional variant thereof encodes a transposase, meganuclease, or Cas protein; and wherein the first gene editing enzyme cleavage sequence and a second gene editing enzyme cleavage sequence is a transposase, meganuclease, or Cas protein recognition and cleavage site, respectively. In some embodiments, the nucleic acid molecule, in 5′ to 3′ orientation, comprises: (i) a nucleic acid sequence encoding a gene editing enzyme or functional variant thereof; (ii) a first gene editing enzyme cleavage sequence; (iii) a barcode sequence; (iv) a second gene editing enzyme cleavage sequence. In some embodiments, the regulatory sequence is positioned with about 50 nucleotides upstream from the 5′ end of the nucleic acid sequence encoding a gene editing enzyme or functional variant thereof. In some embodiments, the regulatory sequence is a cytomegalovirus promoter. In some embodiments, the nucleic acid sequence encoding a gene editing enzyme or functional variant thereof encodes a transposase or functional variant thereof. In some embodiments, the composition furthers comprises a transposase enzyme or a nucleic acid sequence encoding a transposase. In some embodiments, the transposase enzyme is SleepingBeauty, PiggyBac, or a functional variant thereof. In some embodiments, the composition relates to the nucleic acid molecule of encodes a modified transposase enzyme is a modified Sleeping Beauty, SB100×. In some embodiments, the nucleic acid encoding a gene editing enzyme or functional variant thereof comprises a polyA tail sequence. In some embodiments, the composition relates to the nucleic acid molecule comprising a polyA tail sequence that is an SV40 polyA tail.

In some embodiments, the nucleic acid molecule comprises a first and second inverted tandem repeat sequence positioned at the 5′ end and 3′ end of the nucleic acid molecule, respectively. In some embodiments, the first and second inverted tandem repeat sequence comprises an AAV ITR sequence.

Embodiments of the disclosure also relate to a composition comprising a nucleic acid molecule, wherein the nucleic acid molecule is positioned in a liposome, cell or virus. In some embodiments, the nucleic acid molecule is encapsulated within a virus. In some embodiments, the viral particle is an AAV pseudotyped particle. In some embodiments, the composition comprises any of the disclosed nucleic acid molecules, wherein, in 5′ to 3′ orientation, the nucleic acid molecule comprises: (i) a nucleic acid sequence encoding a gene editing enzyme or functional variant thereof; (ii) a first gene editing enzyme cleavage sequence; (iii) a barcode sequence; (iv) a second gene editing enzyme cleavage sequence; wherein the nucleic acid sequence encoding a gene editing enzyme or functional variant thereof is a SleepingBeauty sequence; a first gene editing enzyme cleavage sequence comprises a SleepingBeauty cleavage sequence; and wherein each of (i), (ii), (iii), and (iv) are positioned between a first and second viral ITR. In some embodiments, the viral ITR is an AAV ITR. And, in some embodiments, the nucleic acid sequence that encodes a gene editing enzyme or functional variant thereof is operably linked to a regulatory sequence also part of the nucleic acid molecule. In some embodiments, the barcode and the nucleic acid sequence encoding the gene editing enzyme or the functional variant thereof are on two separate nucleic acid molecules The disclosure relates to a composition comprising a viral vector comprising the nucleic acid molecule of any of the above disclosed embodiments. In some embodiments, the composition relates to the composition comprises a viral vector, wherein the viral vector is an AAV vector. In some embodiments, the viral vector comprises a capsid protein comprising a targeting domain. In some embodiments, the targeting domain associates with an amino acid sequence on a B cell, a T cell or a NK cell. In some embodiments, the viral vector is replication deficient. In some embodiments, the composition relates to the composition of any of claim 19 or 20, wherein the targeting domain comprises a viral AAV VP protein or a functional variant thereof. In some embodiments, the targeting domain comprises AAV VP1, VP2 and/or VP3 or a functional variant thereof. In some embodiments, the targeting domain comprises a ligand and ligand binding partner capable of associating with the ligand. In some embodiments, the targeting domain comprises a chimeric protein, wherein the chimeric protein comprises a first domain that associates with the protein component incorporated in the viral capsid and a second domain comprising an antigen. In some embodiments the viral capsid and/or the antigen directs infection of a cell.

In some embodiments, the viral particle comprises a targeting domain with more than one peptide positioned on the outside of the viral particle, the targeting domain directing association and subsequent entry of the viral particle into a target cell. In some embodiments, the targeting domain is positioned within the GH2 loop or GH3 loop. In some embodiments, the targeting domain comprises a SpyCatcher or a functional variant thereof. In some embodiments, the targeting domain comprises SpyCatcher, SpyTag, one or more antibody fragments, biotin or another peptide optionally covalently or non-covalently bound to an antigen or antigen domain. In some embodiments, the viral vector is an AAV vector that comprises a capsid comprising VP1, VP2 and VP3 amino acids; and wherein the amino acid sequence of at least one VP protein comprises a mutation within a VP2/3 splice acceptor site. In some embodiments, the targeting domain is positioned within the GH2 loop or GH3 loop and wherein the targeting domain comprises an amino acid the associates with or binds a B cell. In some embodiments, the B cell is a human B cell. In some embodiments, the B cell is an antigen-specific plasma cell. The disclosure relates to a composition comprising a cell comprising the nucleic acid molecule disclosed herein. In some embodiments, the cell is a B cell. In some embodiments, the cell is a human cell. In some embodiments, the cell comprises a nucleic acid encoding a gene-editing enzyme and the barcode is integrated within the genomic DNA of the cell.

In some embodiments, the composition relates to a kit comprising: (i) the nucleic acid molecule disclosed herein; and (ii) a nucleic acid molecule encoding a viral vector comprising a capsid protein comprising a targeting domain. In some embodiments, the kit further comprises a helper plasmid. In some embodiments, the kit further comprises a transfection buffer.

The disclosure also relates to a method of manufacturing the composition comprising a cell comprising: transfecting a cell line with nucleic acid sequence encoding a gene-editing enzyme and/or the nucleic acid molecule comprising a barcode positioned between a gene-editing recognition site.

The disclosure also relates to a method of labeling a cell with a barcode comprising: (a) exposing a cell with the composition comprising a nucleic acid molecule disclosed herein for a time period sufficient for the nucleic acid molecule within the viral vector to enter the cell; (b) exposing the nucleic acid sequence encoding the barcode region to the gene editing enzyme for a time period sufficient to cleave the barcode region from the nucleic acid molecule at the gene editing cleavage sites; and (c) allowing a time period sufficient to allow integration of the barcode into genomic DNA of the cell. In some embodiments, the method further comprises exposing the genomic DNA of the cell to a gene editing enzyme after step (b) during the time period of step (c). In some embodiments, the method further comprises: culturing the cell for a time period for clonal expansion of the cell. In some embodiments, the method further comprises a step of detecting the presence of the barcode domain in the genomic DNA of the cell by exposing the barcode domain to a probe specific for the barcode domain sequence. In some embodiments, the cell is an immune cell. In some embodiments, the method relates to the method of any of claims 41 through 46, wherein the cell comprises a target protein or target protein complex on its surface that associates with the targeting domain on any of the disclosed viral particles. In some embodiments, the target protein is chosen from: CD27, CD154, CD19, CD20, CD21, CD40, MHC II, and B7. In some embodiments, the barcode region integrates within the genomic DNA at a site of a nucleic acid sequence that encodes one or a plurality of antibodies or antibody fragments. In some embodiments, the method relates to the method further comprises a step of exposing the cell to an antigen for a time sufficient to elicit an antigen-specific immune response in the cell. In some embodiments, steps (a) through (c) do not induce a measurable immune response against the barcode domain of DNA or the cell is free of an immune response marker specific for the barcode domain. In some embodiments, the time period sufficient for the nucleic acid molecule within the viral vector to enter the cell is no more than about 4 to about 10 minutes. In some embodiments, the time period sufficient to cleave the barcode region from the nucleic acid molecule at the gene editing cleavage sites is no more than about 2 to about 5 minutes. In some embodiments, the time period sufficient to allow integration of the barcode into genomic DNA of the cell is no more than about 1 to about 10 minutes. In some embodiments, the method relates to the method of claim 54 further comprising a step of allowing the gene editing enzyme to be expressed by the nucleic acid molecule cell after step (a). The disclosure relates to a method of identifying immune cell reactivity to an epitope comprising (a) exposing an immune cell to the composition or viral particle disclosed herein for a time period sufficient for the barcode region to integrate within genomic DNA of the immune cell; (b) exposing an antigen comprising an epitope to the immune cell for a time period sufficient to elicit an epitope-specific immune response; (c) identifying the immune cell reactivity to the epitope by correlating the epitope-specific immune response to the presence of the barcode region in the genomic DNA of the immune cell. In some embodiments, step of identifying the immune cell reactivity comprises identifying the sequence of the barcode domain by sequencing the barcode domain or detecting a probe associated with the barcode domain in the genomic DNA. In some embodiments, the immune cell is a T cell or a B cell. In some embodiments, barcode domain integrates into a nucleic acid sequence within the genomic DNA of the cell that encodes an antibody or portion of an antibody. In some embodiments, the nucleic acid sequence within the genomic DNA of the cell that encodes an antibody or portion of an antibody specific for the epitope. In some embodiments, the epitope comprises an amino acid sequence from the targeting domain. In some embodiments, the epitope-specific immune response is the activation of a B cell by stimulation of expression of an antibody or antibody fragment that associates with an epitope of the antigen. In some embodiments, the epitope comprises at least about 5 amino acids from a viral protein. In some embodiments, the viral protein is HIV-1 gp120, human influenza HA, or SARS-CoV2 spike protein. In some embodiments, the method further comprises a step of sequencing the antibody or antibody fragment from the immune cell by PCR or a sequencing reaction and identifying the immune cell reactivity by correlating the antibody or antibody fragment sequence to detection of or sequence of the barcode domain. In some embodiments, the immune cell expresses Spytag and the viral vector expresses SpyCatcher, or respective functional variants thereof. In some embodiments, the composition comprises a viral vector that is an AAV vector comprising a mutation in VP1, VP2, and/or VP3 rendering the AAV infection- and replication-deficient. In some embodiments, the VP1, VP2, and/or VP3 comprise a chimeric structure in which the amino acid sequence comprises an epitope or antigen sequence. In some embodiments, the method further comprises a step of isolating the immune cell by cell sorting after step (b).

The disclosure relates to a method of isolating an immune cell reactive to an epitope comprising: (a) exposing an immune cell to any one or more of the disclosed compositions for a time period sufficient for the barcode region to integrate within genomic DNA of the immune cell; (b) exposing an antigen comprising an epitope to the immune cell for a time period sufficient to elicit an epitope-specific immune response; (c) isolating the immune cell after steps (a) and (b). In some embodiments, the method relates to the method claim 70, wherein the step of isolating is performed by cell sorting after culturing the cell for at least about 2 days. In some embodiments, the method further comprises identifying immune cell reactivity to the epitope by correlating feature of immune reactivity to the epitope sequence. In some embodiments, the method relates to the method of claim 72, wherein the feature of immune reactivity is expression of an antibody or antibody fragment that associates with the epitope and wherein the step of identifying comprises one or a combination of: (i) sequencing the amino acid sequence of the antibody or antibody fragment; (ii) sequencing the nucleic acid sequence encoding the amino acid sequence of the antibody or antibody fragment. In some embodiments, the method relates to the method further comprises a step of identifying the immune cell by detecting the presence of the barcode domain. In some embodiments, the step of detecting is performed by polymerase chain reaction or sequencing. In some embodiments, the immune cell is a T cell or a B cell. In some embodiments, the barcode domain or a portion thereof integrates into a nucleic acid sequence within the genomic DNA of the cell that encodes an antibody or portion of an antibody. In some embodiments, the nucleic acid sequence within the genomic DNA of the cell that encodes an antibody or portion of an antibody specific for the epitope. In some embodiments, the epitope comprises an amino acid sequence from the targeting domain. In some embodiments, the epitope-specific immune response is the activation of a B cell by stimulation of expression of an antibody or antibody fragment that associates with the epitope. In some embodiments, the epitope comprises at least about 5 amino acids from a viral protein. In some embodiments, the viral protein is HIV-1 gp120, human influenza HA, or SARS-CoV2 spike protein. In some embodiments, the method relates to the method of claim 80 further comprising a step of sequencing the antibody or antibody fragment from the immune cell and identifying the immune cell reactivity by correlating the antibody or antibody fragment sequence to detection of or sequence of the barcode domain. In some embodiments, the immune cell expresses Spytag and/or the viral vector expresses SpyCatcher, or respective functional variants thereof. In some embodiments, the composition comprises a viral vector that is an AAV vector comprising a mutation in VP1, VP2, and/or VP3 rendering the AAV infection and replication deficient. In some embodiments, the VP1, VP2, and/or VP3 comprise an amino acid sequence comprising the epitope. In some embodiments, the method relates to the method of identifying an antibody sequence or antibody fragment sequence that associates with a target epitope comprising: (a) exposing an immune cell to the composition for a time period sufficient for the barcode region to integrate within genomic DNA of the immune cell; (b) exposing an antigen comprising an epitope to the immune cell for a time period sufficient to elicit an epitope-specific immune response; (c) isolating the immune cell after steps (a) and (b); and (d) sequencing the antibody sequence or antibody fragment sequence that associates with the target epitope.

### BRIEF DESCRIPTION OF THE DRAWINGS

FIGS. 1A and 1B show a scheme for sequential immunization. FIG. 1A, shows a graphical representation of the process of antibody maturation upon sequential immunization. FIG. 1B shows a comparison of the monoclonal and polyclonal B cell repertoires of Ig KI mice and wild type mice respectively.

FIG. 2A, is a graphical representation of an aptamer-antigen interaction. FIG. 2B is a, diagram describing the SELEX method for aptamer selection.

FIGS. 3A through 3D show that barcodes are internalized by B cells through the BCR and can be detected inside the cell. FIG. 3A: Graphical representation of the method used to evaluate BCR-mediated internalization of a random DNA oligonucleotide by NP-specific B cells.

FIG. 3B: Flow cytometry analysis of NP-specific B cells stimulated for 30 minutes as indicated on the figure. FIG. 3C: Graphical representation of the method used to confirm aptamer internalization through BCR. FIG. 3D, Flow cytometry plot showing fluorescein and kappa light chain (Ig Kappa) expression in Env-specific B cells, 2 hours after receiving the indicated stimuli for 30 min.

FIG. 4 shows a map of the engineered AAV-SB-barcode system designed to permanently barcode antigen-specific B cells. ITR=inverted terminal repeat; CMV=cytomegalovirus promoter; SB-100×=transposase; SB IR/DR=SB recognition sites. FIG. 5 depicts expression of SB100× in 293AAV cells upon transfection with the AAV-SB construct shown in FIG. 1 by Western blot.-indicates control. Actin is loading control.

FIGS. 6A and 6B shows a schematic of antigen pseudotyped AAV. FIG. 6A shows a schematic of an AAV particle expressing SpyCatcher on its surface which is used to establish a covalent bond with a Spytagged antigen. FIG. 6B shows an overview of the AAV-mediated barcoding system of antigen-specific B cells.

FIG. 7 shows a strategy to engineer a pseudotyped and non-infectious AAV. Top scheme represents the wild type AAV capsid gene highlighting the transcription start site for the capsid protein VP1 and the splice acceptor site for the VP2 and VP3 proteins. Also this scheme shows a 7AA site of the capsid proteins that is exposed and has been previously engineered by others and two key residues for AAV binding to heparan sulfate (R585 and R588). The construct in the middle represents our new construct designed to encode the VP1 protein fused to SpyCatcher and with mutations R585A and R588A to abrogate binding to heparan sulfate. The scheme at the bottom represents the construct encoding VP2 and VP3 with the R585A and R588A mutations. FIG. 8A through 8C shows a functional SpyCatcher is expressed on the surface of engineered AAVs. In FIG. 8A, Anti-SpyCatcher monoclonal antibody (mAb) binds to AAV particles with SpyCatcher version 3-containing (SC3), or original SpyCatcher-containing (SC) capsids. but does not bind to AAVs with wildtype (WT) capsid. Positive control is SpyCatcher-displaying Virus Like Particles. FIG. 8B shows AAVs with SC3 or SC on their capsid exhibit robust binding to two different SpyTagged HIV Envelope immunogens, but not to non-SpyTagged immunogens, suggesting that the SpyCatcher displayed on the AAV surface is functional and able to bind SpyTagged proteins. FIG. 8C shows nsEM images showing conjugation of a Spy Tagged HIV envelope trimer (RC1) to the surface of our engineered AAVs. Anti-SpyCatcher monoclonal antibody binding to AAV particles with wildtype (WT), SpyCatcher3-containing (SC3), or SpyCatcher-containing (SC) capsids. Positive control is SpyCatcher-displaying VLPs. No anti-SpyCatcher antibody binding is observed to the WT capsid, however there is detectable binding to both SC3 and SC capsids. Additionally, there is no difference in OD405 absorbance with WT capsid-expressing AAVs are incubated with anti-SpyCatcher antibody or a vehicle control, suggesting the antibody is highly specific for SpyCatcher. b, AAVs with WT, SC3, or SC capsids were incubated with proteins containing SpyTags or non-SpyTagged controls. For AAVs with WT capsids, very low binding is observed to SpyTagged proteins and non-Spytagged proteins alike. However, AAVs with SC3 or SC on their capsid exhibit robust binding to two different SpyTagged HIV Envelope immunogens, but not to non-Spy Tagged immunogens, suggesting that the SpyCatcher displayed on the AAV surface is functional and able to bind SpyTagged proteins. This will allow for a modular system whereby various Spytagged antigens can be conjugated to the AAV-SC capsid.

FIGS. 9A and 9B depict a Splinkerette PCR confirming the persistence of integrated barcodes for more than 50 days and enabling integration site discrimination. 293T cells were infected with AAV-SB, passaged for the indicated number of days, and then DNA was extracted and subjected to splinkerette PCR. One strong band was identified potentially suggesting that a single integration event in one cell during AAV-SB infection allowed it to outcompete other cells and become the dominant clone. 9B depicts Sanger sequencing of the splinkerette product revealed homology to a region of chromosome 15 (“red rectangle” is equivalent to the first box surrounding the 5′ most portion of the DNA sequence encompassing over 8 horizontal lines of DNA code on the Figure), followed by part of the AAV IR/DR (“purple rectangle” is equivalent to the box drawn around the portion of DNA corresponding with the amino acid sequence beginning with HSL) and the engineered unique barcode (“pink rectangle” is equivalent to the box drawn around the portion of DNA in the most 3′ position relative to the sequence of the FIG corresponding with the amino acid sequence beginning with LDC). 293T cell cultures were passaged for 6 and 56 days. The PCR band marked with a asterisk was purified and an integration site in chromosome 11 was identified. FIG. 9B shows that one strong band (red circle) was identified after splinkerette PCR using genomic DNA from a third cell culture. FIG. 10 depicts an illustration of a modified AAV particle capable of targeting and labeling a B cell.

FIG. 11 depicts an illustration of a modified AAV particle capable of targeting and labeling a T cell. In this embodiment, the viral particle comprises a MHC II complex on its surface (with both MHC II associated with PADRE); and the T cell comprises a TCR that mediates entry of the AAV particle through its association with PADRE and MHCII.

MHC II amino acid sequence:
MHRRRSRSCR EDQKPVMDDQ RDLISNNEQL PMLGRRPGAP
ESKCSRGALY TGFSILVTLL LAGQATTAYF LYQQQGRLDK
LTVTSQNLQL ENLRMKLPKP PKPVSKMRMA TPLLMQALPM
GALPQGPMQN ATKYGNMTED HVMHLLQNAD PLKVYPPLKG
SFPENLRHLK NTMETIDWKV FESWMHHWLL FEMSRHSLEQ
KPTDAPPKVL TKCQEEVSHI PAVHPGSFRP KCDENGNYLP
LQCYGSIGYC WCVFPNGTEV PNTRSRGHHN CSESLELEDP
SSGLGVTKQD LGPVPM

FIG. 12 depicts an illustration of a modified AAV particle capable of targeting and labeling a cancer cell. The viral particle comprises EGF on its surface, which mediates entry into the cancer cell expressing EGFR.

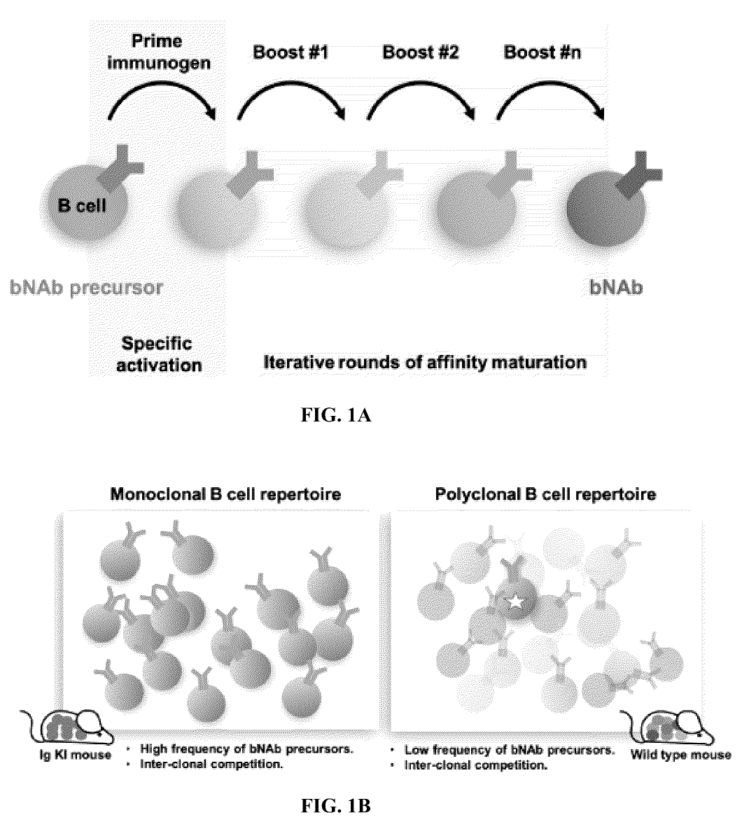

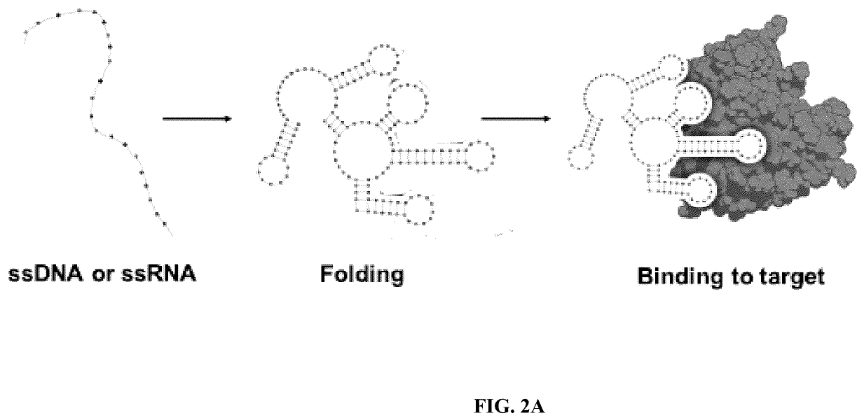

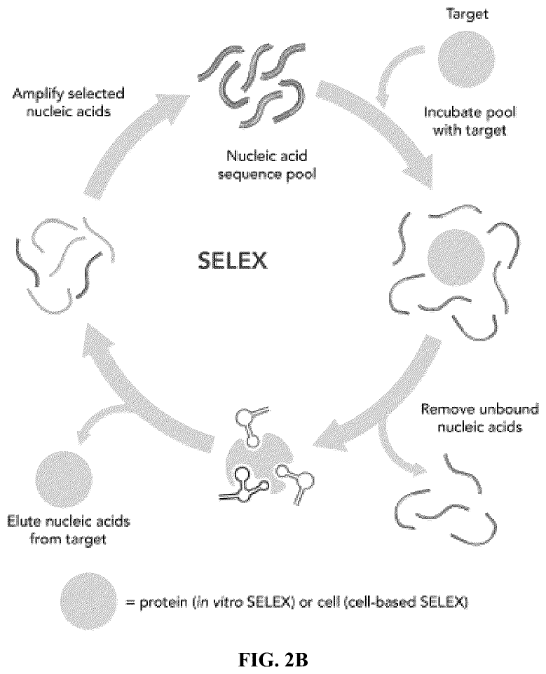

EGF amino acid sequence:
MLLTLIILLP VVSKFSFVSL SAPQHWSCPE GTLAGNGNST
CVGPAPFLIF SHGNSIFRID TEGTNYEQLV VDAGVSVIMD
FHYNEKRIYW VDLERQLLQR VFLNGSRQER VCNIEKNVSG
MAINWINEEV IWSNQQEGII TVTDMKGNNS HILLSALKYP
ANVAVDPVER FIFWSSEVAG SLYRADLDGV GVKALLETSE
KITAVSLDVL DKRLFWIQYN REGSNSLICS CDYDGGSVHI
SKHPTQHNLF AMSLFGDRIF YSTWKMKTIW IANKHTGKDM
VRINLHSSFV PLGELKVVHP LAQPKAEDDT WEPEQKLCKL
RKGNCSSTVC GQDLQSHLCM CAEGYALSRD RKYCEDVNEC
AFWNHGCTLG CKNTPGSYYC TCPVGFVLLP DGKRCHQLVS
CPRNVSECSH DCVLTSEGPL CFCPEGSVLE RDGKTCSGCS
SPDNGGCSQL CVPLSPVSWE CDCFPGYDLQ LDEKSCAASG
PQPFLLFANS QDIRHMHFDG TDYGTLLSQQ MGMVYALDHD
PVENKIYFAH TALKWIERAN MDGSQRERLI EEGVDVPEGL
AVDWIGRRFY WTDRGKSLIG RSDLNGKRSK IITKENISQP
RGIAVHPMAK RLFWTDTGIN PRIESSSLQG LGRLVIASSD
LIWPSGITID FLTDKLYWCD AKQSVIEMAN LDGSKRRRLT
QNDVGHPFAV AVFEDYVWFS DWAMPSVMRV NKRTGKDRVR
LQGSMLKPSS LVVVHPLAKP GADPCLYQNG GCEHICKKRL
GTAWCSCREG FMKASDGKTC LALDGHQLLA GGEVDLKNQV
TPLDILSKTR VSEDNITESQ HMLVAEIMVS DQDDCAPVGC
SMYARCISEG EDATCQCLKG FAGDGKLCSD IDECEMGVPV
CPPASSKCIN TEGGYVCRCS EGYQGDGIHC LDIDECQLGE
HSCGENASCT NTEGGYTCMC AGRLSEPGLI CPDSTPPPHL
REDDHHYSVR NSDSECPLSH DGYCLHDGVC MYIEALDKYA
CNCVVGYIGE RCQYRDLKWW ELRHAGHGQQ QKVIVVAVCV
VVLVMLLLLS LWGAHYYRTQ KLLSKNPKNP YEESSRDVRS
RRPADTEDGM SSCPQPWFVV IKEHQDLKNG GQPVAGEDGQ
AADGSMQPTS WRQEPQLCGM GTEQGCWIPV SSDKGSCPQV
MERSFHMPSY GTQTLEGGVE KPHSLLSANP LWQQRALDPP
HQMELTQ
EGFR amino acid sequence:
MRPSGTAGAA LLALLAALCP ASRALEEKKV CQGTSNKLTQ
LGTFEDHFLS LQRMFNNCEV VLGNLEITYV QRNYDLSFLK
TIQEVAGYVL IALNTVERIP LENLQIIRGN MYYENSYALA
VLSNYDANKT GLKELPMRNL QGQKCDPSCP NGSCWGAGEE
NCQKLTKIIC AQQCSGRCRG KSPSDCCHNQ CAAGCTGPRE
SDCLVCRKFR DEATCKDTCP PLMLYNPTTY QMDVNPEGKY
SFGATCVKKC PRNYVVTDHG SCVRACGADS YEMEEDGVRK
CKKCEGPCRK VCNGIGIGEF KDSLSINATN IKHFKNCTSI
SGDLHILPVA FRGDSFTHTP PLDPQELDIL KTVKEITGFL
LIQAWPENRT DLHAFENLEI IRGRTKQHGQ FSLAVVSLNI
TSLGLRSLKE ISDGDVIISG NKNLCYANTI NWKKLFGTSG
QKTKIISNRG ENSCKATGQV CHALCSPEGC WGPEPRDCVS
CRNVSRGREC VDKCNLLEGE PREFVENSEC IQCHPECLPQ
AMNITCTGRG PDNCIQCAHY IDGPHCVKTC PAGVMGENNT
LVWKYADAGH VCHLCHPNCT YGCTGPGLEG CPTNGPKIPS
IATGMVGALL LLLVVALGIG LFMRRRHIVR KRTLRRLLQE
RELVEPLTPS GEAPNQALLR ILKETEFKKI KVLGSGAFGT
VYKGLWIPEG EKVKIPVAIK ELREATSPKA NKEILDEAYV
MASVDNPHVC RLLGICLTST VQLITQLMPF GCLLDYVREH
KDNIGSQYLL NWCVQIAKGM NYLEDRRLVH RDLAARNVLV
KTPQHVKITD FGLAKLLGAE EKEYHAEGGK VPIKWMALES
ILHRIYTHQS DVWSYGVTVW ELMTFGSKPY DGIPASEISS
ILEKGERLPQ PPICTIDVYM IMVKCWMIDA DSRPKFRELI
IEFSKMARDP QRYLVIQGDE RMHLPSPTDS NFYRALMDEE
DMDDVVDADE YLIPQQGFFS SPSTSRTPLL SSLSATSNNS
TVACIDRNGL QSCPIKEDSF LQRYSSDPTG ALTEDSIDDT
FLPVPGEWLV WKQSCSSTSS THSAAASLQC PSQVLPPASP
EGETVADLQTQ

### DETAILED DESCRIPTION OF EMBODIMENTS

The disclosure provides a new technique to track antigen-specific B cells upon multiple repeated immunizations. Tracking antigen specific B cells responding to sequential immunization will illuminate the mechanisms of epitope immunodominance, memory B cell reactivation and B cell fate into the memory and plasma cell compartments.

This disclosure will advance novel approaches for vaccine design using a groundbreaking technology to silence immunodominant epitopes and track the history of antigen-B cell encounters upon sequential immunization in vivo.

The terminology used herein is for the purpose of describing particular embodiments only and is not intended to be limiting. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the disclosure described herein are capable of operation in other sequences than described or illustrated herein.

The following terms or definitions are provided solely to aid in the understanding of the disclosure. Unless specifically defined herein, all terms used herein have the same meaning as they would to one skilled in the art of the present disclosure. Practitioners are particularly directed to Sambrook et al., Molecular Cloning: A Laboratory Manual, 2nd ed., Cold Spring Harbor Press, Plainsview, N.Y. (1989); and Ausubel et al., Current Protocols in Molecular Biology (Supplement 47), John Wiley & Sons, New York (1999), for definitions and terms of the art. The definitions provided herein should not be construed to have a scope less than understood by a person of ordinary skill in the art.

As used in the specification and the appended claims, the singular forms “a,” “an” and “the” include plural referents unless the context clearly dictates otherwise.

“AAV virion” refers to a complete virus particle, such as for example a wild type AAV virion particle, which comprises single stranded genome DNA packaged into AAV capsid proteins. The single stranded nucleic acid molecule is either sense strand or antisense strand, as both strands are equally infectious. A “rAAV virion” refers to a recombinant AAV virus particle, i.e. a particle which is infectious but replication defective. It is composed of an AAV protein shell and comprises a rAAV vector. In the context of the present disclosure the protein shell may be of a different serotype than the rAAV vector. An AAV virion of the disclosure may thus be composed a protein shell, i.e. the icosahedral capsid, which comprises capsid proteins (VP1, VP2, and/or VP3) of one AAV serotype, e.g. AAV serotype 6, whereas the rAAV vector contained in that AAV6 virion may be any of the rAAVX vectors described above, including a rAAV6 vector. An “rAAV6 virion” comprises capsid proteins of AAV serotype 6, while e.g. a rAAV2 virion comprises capsid proteins of AAV serotype 2, whereby either may comprise any of rAAVX vectors of the disclosure. “AAV helper functions” generally refers to the corresponding AAV functions required for rAAV replication and packaging supplied to the rAAV virion or rAAV vector in trans. AAV helper functions complement the AAV functions which are missing in the rAAV vector, but they lack AAV ITRs (which are provided by the rAAV vector). AAV helper functions include the two major ORFs of AAV, namely the rep coding region and the cap coding region or functional substantially identical sequences thereof. Rep and Cap regions are well known in the art, see e.g. Chiorini et al. (1999, J. of Virology, Vol 73 (2): 1309-1319) or U.S. Pat. No. 5,139,941, incorporated herein by reference. The AAV helper functions can be supplied on a AAV helper construct. Introduction of the helper construct by into the host cell can occur e.g. by transformation or transduction prior to or concurrently with the introduction of the rAAV vector. The AAV helper constructs of the disclosure may thus be chosen such that they produce the desired combination of serotypes for the rAAV virion's capsid proteins on the one hand and for the rAAV vector replication and packaging on the other hand.

“AAV helper virus” provides additional functions required for AAV replication and packaging. Suitable AAV helper viruses include adenoviruses, herpes simplex viruses (such as HSV types 1 and 2) and vaccinia viruses. The additional functions provided by the helper virus can also be introduced into the host cell via vectors, as described in U.S. Pat. No. 6,531,456 incorporated herein by reference.

The term “about” as used herein when referring to a measurable value such as an amount, a temporal duration, and the like, is meant to encompass variations of ±20%, ±10%, ±5%,, ±1%, or ±0.1% from the specified value, as such variations are appropriate to perform the disclosed methods. For recitation of numeric ranges herein, each intervening number therebetween with the same degree of precision is explicitly contemplated. For example, for the range of 6-9, the numbers 7 and 8 are contemplated in addition to 6 and 9, and for the range from about 6.0 to about 7.0, the numbers 6.0, 6.1, 6.2, 6.3, 6.4, 6.5, 6.6, 6.7, 6.8, 6,9, and 7.0 are explicitly contemplated.

The term “antibody”, as used herein, broadly refers to any immunoglobulin (Ig) molecule comprised of four polypeptide chains, two heavy (H) chains and two light (L) chains, or any functional variant, mutant, variant, or derivative thereof, which retains the essential epitope binding features of an Ig molecule. Such mutant, variant, or derivative antibody formats are known in the art. Non-limiting embodiments of which are discussed below. In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. Immunoglobulin molecules can be of any type (e.g., IgG, IgE, IgM, IgD, IgA and IgY), class (e.g., IgG 1, IgG2, IgG 3, IgG4, IgA1 and IgA2) or subclass.

The term “antigen binding portion” or “antigen binding fragment” of an antibody (or simply “antibody portion” or “antibody fragment”), as used herein, refers to one or more fragments of an antibody that retain the ability to specifically bind to an antigen. It has been shown that the antigen-binding function of an antibody can be performed by fragments of a full-length antibody. Such antibody embodiments may also be bispecific, dual specific, or multi-specific formats; specifically binding to two or more different antigens. Examples of binding fragments encompassed within the term “antigen-binding portion” or “antigen binding fragment” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546, Winter et al., PCT publication WO 90/05144 A1 herein incorporated by reference), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” or “antigen binding fragment” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123). Such antibody binding portions are known in the art (Kontermann and Dubel eds., Antibody Engineering (2001) Springer-Verlag. New York. 790 pp. (ISBN 3-540-41354-5). Antibody fragments also include “nanobody” or “nanobodies” which are a polypeptide that consist of the variable domain of heavy-chain-only antibody. The structure was first isolated two decades ago from the serum of Camelidae family. In some embodiments, the nanobody comprises a length of about 4 nm, and a width of about 2.5 nm, and a weight of only about 14 kD to about 16 kD in molecular weight. The antigen-binding capacity of nanobodies, however, remains similar to that of conventional antibodies for the following reasons. First, the complementarity-determining region 3 (CDR3) of nanobodies is similar or even longer than that of human VH domain (variable domain of heavy immunoglobulin chain). In some embodiments, the CDR of a nanobody consists of from about 3 to about 28 amino acids in length. Second, nanobodies can form finger-like structures to recognize cavities or hidden epitopes that are not available to mAbs. This feature not only enhances the binding affinity and specificity of nanobodies, but also enables the discovery of novel pharmacological targets including the receptor-binding pockets or enzymatic active sites. Third, nanobodies of the disclosure may exhibit excellent stability, hydrophilicity, and water solubility that help maintain their binding affinity across different conditions, which can be further reinforced by mutating key AAs in the framework region. In some embodiments, viral particles of the disclosure comprise a modified VP1, VP2 and VP3 AAV capsid proteins but display one or more nanobodies on their surface in associate with an antigen. Manufacture of nanobodies is generally known. The protocols necessary to manufacture and use nanobodies are disclosed in Koch, t al., Sci Rep. 2017; 7:8390, which is incorporated by reference in its entirety.

The term “antigen” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. The term “antigen” can also refer to a molecule that an antibody or antibody-like molecule can bind to or is recognized by the antibody or antibody-like molecule.

“Cell type” means the organism, organ, and/or tissue type from which the cell is derived or sourced, state of development, phenotype or any other categorization of a particular cell that appropriately forms the basis for defining it as “similar to” or “different from” another cell or cells.

“Coding sequence” or “encoding nucleic acid” as used herein may mean refers to the nucleic acid (RNA, DNA, or RNA/DNA hybrid molecule) that comprises a nucleotide sequence which encodes a protein. The coding sequence may further include initiation and termination signals operably linked to regulatory elements including a promoter and polyadenylation signal capable of directing expression in the cells of an individual or mammal to whom the nucleic acid is administered.

“Complement” or “complementary” as used herein may mean a nucleic acid may mean Watson-Crick (e.g., A-T/U and C-G) or Hoogsteen base pairing between nucleotides or nucleotide analogs of nucleic acid molecules.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. As used herein, a vector that encodes a protein of interest refers to a vector containing a nucleotide sequence that encodes for that protein or proteins.

As used herein, the term “exogenous” refers to any material introduced from or produced outside an organism, cell, tissue or system. A protein that is referred to as a heterologous protein is expressed by exogenous material (vectors, nucleotide sequences, and the like) that has been introduced into the organism, cell, tissue or system. For the avoidance of doubt, a heterologous protein, a heterologous vector, or heterologous nucleotide molecule is not the same as that may present in the native, unmodified genome of the organism, cell, tissue or system. A heterologous protein, vector, or nucleotide sequence that may have the same or similar to a sequence already present in the organism, cell, tissue, or system, is expressed from a location or sequence that is other than the native sequence found in the genome of that organism, cell, tissue, or system

As used herein, the term “functional variant” means any portion of a polypeptide that is of a sufficient length to retain at least partial biological function that is similar to or substantially similar to the wild-type polypeptide upon which the fragment is based. In some embodiments, a functional variant of a polypeptide is a polypeptide that comprises or possesses 80, 85, 90, 95, 96, 97, 98, or 99% sequence identity to any polypeptide disclosed in Table F and has sufficient length to retain at least partial binding affinity to one or a plurality of ligands that bind to the polypeptides in Table F. In some embodiments, a functional variant of a nucleic acid is a nucleic acid that comprises or possesses 80, 85, 90, 95, 96, 97, 98, or 99% sequence identity to any nucleic acid to which it is being compared and has sufficient length to retain at least partial function related to the nucleic acid to which it is being compared. In some embodiments, the fragment is a fragment of any polypeptide disclosed in Table F and has a length of at least about 10, about 20, about 30, about 40, about 50, about 60, about 70, about 80, about 90, or about 100 contiguous amino acids. In some embodiments, the fragment is a fragment of any polypeptide disclosed in Table F and has a length of at least about 50 amino acids. In some embodiments, the fragment is a fragment of any polypeptide disclosed in Table F and has a length of at least about amino acids. In some embodiments, the fragment is a fragment of any polypeptide disclosed in Table F and has a length of at least about 150 amino acids. In some embodiments, the fragment is a fragment of any polypeptide disclosed in Table F and has a length of at least about amino acids. In some embodiments, the fragment is a fragment of any polypeptide disclosed in Table F and has a length of at least about 250 amino acids. In some embodiments, the fragment is a fragment of any polypeptide disclosed in Table F and has a length of at least about amino acids. In some embodiments, the fragment is a fragment of any polypeptide disclosed in Table F and has a length of at least about 350 amino acids. In some embodiments, the fragment is a fragment of any polypeptide disclosed in Table F and has a length of at least about amino acids. In some embodiments, the fragment is a fragment of any polypeptide disclosed in Table F and has a length of at least about 450 amino acids. In some embodiments, the fragment is a fragment of any polypeptide disclosed in Table F and has a length of at least about amino acids. In some embodiments, the fragment is a fragment of any polypeptide disclosed in Table F and has a length of at least about 550 amino acids. In some embodiments, the fragment is a fragment of any polypeptide disclosed in Table F and has a length of at least about amino acids. In some embodiments, the fragment is a fragment of any polypeptide disclosed in Table F and has a length of at least about 650 amino acids. In some embodiments, the fragment is a fragment of any polypeptide disclosed in Table F and has a length of at least about amino acids. In some embodiments, the fragment is a fragment of any polypeptide disclosed in Table F and has a length of at least about 750 amino acids. In some embodiments, the fragment is a fragment of any polypeptide disclosed in Table F and has a length of at least about amino acids. In some embodiments, the fragment is a fragment of any polypeptide disclosed in Table F and has a length of at least about 850 amino acids. In some embodiments, the fragment is a fragment of any polypeptide disclosed in Table F and has a length of at least about amino acids. In some embodiments, the fragment is a fragment of any polypeptide disclosed in Table F and has a length of at least about 950 amino acids. In some embodiments, the fragment is a fragment of any polypeptide disclosed in Table F and has a length of at least about amino acids. In some embodiments, the fragment is a fragment of any polypeptide disclosed in Table F and has a length of at least about 1050 amino acids. In some embodiments, the fragment is a fragment of any polypeptide disclosed in Table F and has a length of at least about 1250 amino acids. In some embodiments, the fragment is a fragment of any polypeptide disclosed in Table F and has a length of at least about 1500 amino acids. In some embodiments, the fragment is a fragment of any polypeptide disclosed in Table F and has a length of at least about 1750 amino acids. In some embodiments, the fragment is a fragment of any polypeptide disclosed in Table F and has a length of at least about 2000 amino acids. In some embodiments, the fragment is a fragment of any polypeptide disclosed in Table F and has a length of at least about 2250 amino acids. In some embodiments, the fragment is a fragment of any polypeptide disclosed in Table F and has a length of at least about 2500 amino acids. In some embodiments, the fragment is a fragment of any polypeptide disclosed in Table F and has a length of at least about 2750 amino acids. In some embodiments, the fragment is a fragment of any polypeptide disclosed in Table F and has a length of at least about 3000 amino acids.

The term “polypeptide” encompasses two or more naturally or non-naturally-occurring amino acids joined by a covalent bond (e.g., an amide bond). Polypeptides as described herein include full-length proteins (e.g., fully processed pro-proteins or full-length synthetic polypeptides) as well as shorter amino acid sequences (e.g., fragments of naturally-occurring proteins or synthetic polypeptide fragments).

As used herein, “sequence identity” is determined by using the stand-alone executable BLAST engine program for blasting two sequences (b12seq), which can be retrieved from the National Center for Biotechnology Information (NCBI) ftp site, using the default parameters (Tatusova and Madden, FEMS Microbiol Lett., 1999, 174, 247-250; which is incorporated herein by reference in its entirety).

The term “subject” is used throughout the specification to describe an animal from which a cell sample is taken or an animal to which a disclosed virus or viral vector has been administered. In some embodiment, the animal is a human. For diagnosis of those conditions which are specific for a specific subject, such as a human being, the term “patient” may be interchangeably used. In some instances in the description of the present disclosure, the term “patient” will refer to human patients suffering from a particular disease or disorder. In some embodiments, the subject may be a mammal which functions as a source of the isolated cell sample. In some embodiments, the subject may be a non-human animal from which a cell sample is isolated or provided, such as a mammal. The term “mammal” encompasses both humans and non-humans and includes but is not limited to humans, non-human primates, canines, felines, murines, bovines, equines, and porcines.

“Nucleic acid” or “oligonucleotide” or “polynucleotide” as used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods. In some embodiments, the nucleic acid is isolated from an organism.

The terms “polynucleotide,” “oligonucleotide” and “nucleic acid” are also used interchangeably throughout and include DNA molecules (e.g., cDNA or genomic DNA), RNA molecules (e.g., mRNA), analogs of the DNA or RNA generated using nucleotide analogs (e.g., peptide nucleic acids and non-naturally occurring nucleotide analogs), and hybrids thereof. The nucleic acid molecule can be single-stranded or double-stranded. In some embodiments, the nucleic acid molecules of the disclosure comprise a contiguous open reading frame encoding an antibody, or a fragment thereof, as described herein. “Nucleic acid” or “oligonucleotide” or “polynucleotide” as used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions. Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine Nucleic acids may be obtained by chemical synthesis methods or by recombinant methods.

A nucleic acid will generally contain phosphodiester bonds, although, in some embodiments, nucleic acid analogs may be included that may have at least one different linkage, e.g., phosphoramidate, phosphorothioate, phosphorodithioate, or O-methylphosphoroamidite linkages and peptide nucleic acid backbones and linkages. Other analog nucleic acids include those with positive backbones; non-ionic backbones, and non-ribose backbones, including those described in U.S. Pat. Nos. 5,235,033 and 5,034,506, which are incorporated by reference in their entireties. Nucleic acids containing one or more non-naturally occurring or modified nucleotides are also included within one definition of nucleic acids. The modified nucleotide analog may be located for example at the 5′-end and/or the 3′-end of the nucleic acid molecule. Representative examples of nucleotide analogs may be selected from sugar- or backbone-modified ribonucleotides. It should be noted, however, that also nucleobase-modified ribonucleotides, i.e. ribonucleotides, containing a non-naturally occurring nucleobase instead of a naturally occurring nucleobase such as uridines or cytidines modified at the 5-position, e.g. 5-(2-amino) propyl uridine, 5-bromo uridine; adenosines and guanosines modified at the 8-position, e.g. 8-bromo guanosine; deaza nucleotides, e.g. 7-deaza-adenosine; O- and N-alkylated nucleotides, e.g. N6-methyl adenosine are suitable. The 2′-OH-group may be replaced by a group selected from H, OR, R, halo, SH, SR, NH₂, NHR, N₂ or CN, wherein R is C₁-C₆ alkyl, alkenyl or alkynyl and halo is F, Cl, Br or I. Modified nucleotides also include nucleotides conjugated with cholesterol through, e.g., a hydroxyprolinol linkage as described in Krutzfeldt et al., Nature (Oct. 30, 2005), Soutschek et al., Nature 432:173-178 (2004), and U.S. Patent Publication No. 20050107325, which are incorporated herein by reference in their entireties. Modified nucleotides and nucleic acids may also include locked nucleic acids (LNA), as described in U.S. Patent Publication No. 20020115080, which is incorporated herein by reference. Additional modified nucleotides and nucleic acids are described in U.S. Patent Publication No. 20050182005, which is incorporated herein by reference in its entirety. Modifications of the ribose-phosphate backbone may be done for a variety of reasons, e.g., to increase the stability and half-life of such molecules in physiological environments, to enhance diffusion across cell membranes, or as probes on a biochip. Mixtures of naturally occurring nucleic acids and analogs may be made; alternatively, mixtures of different nucleic acid analogs, and mixtures of naturally occurring nucleic acids and analogs may be made. In some embodiments, the nucleotide sequence encoding one or more a target protein is free of modified nucleotide analogs. In some embodiments, the nucleotide sequence encoding a target protein comprises from about 1 to about nucleic acid modifications. In some embodiments, the nucleotide sequence encoding one or more target proteins comprises from about 1 to about 50 nucleic acid modifications. In some embodiments, the nucleotide sequence encoding one or more target proteins independently comprise from about 1 to about 100 nucleic acid modifications.

As used herein, the term “nucleic acid molecule” comprises one or more nucleotide sequences that encode one or more proteins. In some embodiments, a nucleic acid molecule comprises initiation and termination signals operably linked to regulatory elements including a promoter and polyadenylation signal capable of directing expression in the cells of the individual to whom the nucleic acid molecule is administered. In some embodiments, the nucleic acid molecule also is a plasmid or vector comprising one or more nucleotide sequences that encode one or plurality or gene-editing enzymes or that comprise a barcode sequence. In some embodiments, the nucleic acid molecule

The terms “polypeptide”, “peptide” and “protein” are used interchangeably herein to refer to polymers of amino acids of any length. The polymer may be linear or branched, it may comprise modified amino acids, and it may be interrupted by non-natural amino acids or chemical groups that are not amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid variants and peptidomimetics.

As used herein, “conservative” amino acid substitutions may be defined as set out in Tables A, B, or C below. The compositions, pharmaceutical compositions and method may comprise nucleic acid sequences comprising one or more conservative substitutions. In some embodiments, the compositions, pharmaceutical compositions and methods comprise nucleic acid sequences that retain from about 70% sequence identity to about 99% sequences identity to the sequence identification numbers disclosed herein but comprise one or more conservative substitutions. Conservative substitutions of the present disclosure include those wherein conservative substitutions (from either nucleic acid or amino acid sequences) have been introduced by modification of polynucleotides encoding polypeptides. Amino acids can be classified according to physical properties and contribution to secondary and tertiary protein structure. A conservative substitution is recognized in the art as a substitution of one amino acid for another amino acid that has similar properties. In some embodiments, the conservative substitution is recognized in the art as a substitution of one nucleic acid for another nucleic acid that has similar properties, or, when encoded, has similar biological effect, such as an antigen binding to the surface receptor of a target cell. Exemplary conservative substitutions are set out in Table A.

TABLE A
Conservative Substitutions I

| | Side Chain | |
|---|---|---|
| Aliphatic | Characteristics | Amino Acid |
| | Non-polar | GAPILVF |
| | Polar-uncharged | C S T M N Q |
| | Polar-charged | D E K R |
| Aromatic | | H F W Y |
| Other | | N Q D E |

Alternately, conservative amino acids can be grouped as described in Lehninger, (Biochemistry, Second Edition; Worth Publishers, Inc. NY, N.Y. (1975), pp. 71-77) as set forth in Table B.

TABLE B
Conservative Substitutions II

| Side Chain Characteristic | Amino Acid |
|---|---|
| Non-polar (hydrophobic): | |
| Aliphatic: | A L I V P |
| Aromatic: | F W Y |
| Sulfur-containing: | M |
| Borderline: | G Y |
| Uncharged-polar | |
| Hydroxyl: | S T Y |
| Amides: | N Q |
| Sulfhydry1: | C |
| Borderline: | G Y |
| Charged (Basic): | K R H |
| Charged (Acidic): | D E |

Alternately, exemplary conservative substitutions are set out in Table C.

TABLE C
Conservative Substitutions III

| Original Residue | Exemplary Substitution |
|---|---|
| Ala (A) | Val Leu Ile Met |
| Arg (R) | Lys His |
| Asn (N) | Gln |
| Asp (D) | Glu |
| Cys (C) | Ser Thr |
| Gln (Q) | Asn |
| Glu (E) | Asp |
| Gly (G) | Ala Val Leu Pro |
| His (H) | Lys Arg |
| Ile (I) | Leu Val Met Ala Phe |
| Leu (L) | Ile Val Met Ala Phe |
| Lys (K) | Arg His |
| Met (M) | Leu Ile Val Ala |
| Phe (F) | Trp Tyr Ile |
| Pro (P) | Gly Ala Val Leu Ile |
| Ser (S) | Thr |
| Thr (T) | Ser |
| Trp (W) | Tyr Phe Ile |
| Tyr (Y) | Trp Phe Thr Ser |
| Val (V) | Ile Leu Met Ala |

It should be understood that the enzymes with mutations (such as the AAV capsid protein or any gene editing enzyme) or any functional variants thereof described herein are intended to include amino acid sequences comprising polypeptides bearing one or more insertions, deletions, or substitutions, or any combination thereof, of amino acid residues as well as modifications other than insertions, deletions, or substitutions of amino acid residues, such as but not limited to conservative amino acid substitutions.

“Operably linked” as used herein may mean that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

“Promoter” as used herein may mean a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which can be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter.

“rAAV vector” as used herein refers to a recombinant vector derived from an adeno-associated virus serotype, such as AAV1, AAV2, AAV3, AAV4, AAV5, AAV6, AAV7, AAV8 and others. rAAV vectors have one or preferably all wild type AAV genes deleted, but still comprise functional ITR nucleic acid sequences. Functional ITR sequences are necessary for the replication, rescue and packaging of AAV virions. The ITR sequences may be wild type sequences or substantially identical sequences (as defined below) or may be altered by for example in insertion, mutation, deletion or substitution of nucleotides, as long as they remain functional. “rAAV vector” as used herein also refers to a recombinant AAV vector comprising the ITR nucleic acid sequences of any of the AAV serotypes, or nucleic acid sequences being substantially identical to the particular AAV serotype wild type ITR sequences, as long as they remain functional. Nucleotide sequences of choice are inserted between the AAV ITR sequences, for example expression constructs comprising an expression regulatory element operably linked to a coding sequence and a 3′ termination sequence. The term “rAAV vector” as used herein also refers to a recombinant AAV vector comprising the ITR nucleic acid sequences of the AAV serotype, or nucleic acid sequences being substantially identical to the AAV serotype wild type ITR sequences, as long as they remain functional. The term “rAAV5 vector” or “rAAV2 vector” is thus used to indicate a rAAV5 or rAAV2 vector comprising respectively the ITR nucleic acid sequences of AAV serotype 5 or serotype 2, or nucleic acid sequences substantially identical thereto. In some embodiments, the viral particle comprises a AAV2 particle comprising an AAV2 VP1 protein and a modified VP2 and VP3 protein that is infection-deficient and chimeric, such that the V2-VP3 protein comprises a protein targeting domain comprising an antigen amino acid sequence optionally positioned distally from the VP3 domain by a peptide sequence responsible for protein-protein interactions of the antigen amino acid sequence to the viral particle.

“Stringent hybridization conditions” as used herein may mean conditions under which a first nucleic acid sequence (e.g., probe) will hybridize to a second nucleic acid sequence (e.g., target), such as in a complex mixture of nucleic acids. Stringent conditions are sequence-dependent and will be different in different circumstances. Stringent conditions may be selected to be about 5-10° C. lower than the thermal melting point (Tₘ) for the specific sequence at a defined ionic strength pH. The Tₘ may be the temperature (under defined ionic strength, pH, and nucleic concentration) at which 50% of the probes complementary to the target hybridize to the target sequence at equilibrium (as the target sequences are present in excess, at Tₘ, 50%> of the probes are occupied at equilibrium). Stringent conditions may be those in which the salt concentration is less than about 1.0 M sodium ion, such as about 0.01-1.0 M sodium ion concentration (or other salts) at pH 7.0 to 8.3 and the temperature is at least about 30° C. for short probes (e.g., about 10-50 nucleotides) and at least about 60° C. for long probes (e.g., greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50%>formamide, 5×SSC, and 1% SDS, incubating at 42° C., or, 5×SSC, 1% SDS, incubating at 65° C., with wash in 0.2×SSC, and 0.1% SDS at 65° C.

“Substantially complementary” as used herein may mean that a first sequence is at least 60%, 65%, 70%, 75%, 80%, 85%, 90%, 95%, 96%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or that the two sequences hybridize under stringent hybridization conditions.

“Substantially identical” as used herein may mean that, in respect to a first and a second sequence, a first and second sequence are at least 60%, 65%, 70%, 75%, 80%, 85%, 90%, 95%, 96%, 97%, 98% or 99% identical over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or with respect to nucleic acids, if the first sequence is substantially complementary to the complement of the second sequence.

“Subtype” or “serotype”: as used herein, interchangeably, and in reference to AAV, means genetic variants of an AAV such that one subtype is less recognized by an immune system of a subject apart from a different subtype. In some embodiments, the viral vector comprises at least one cap polypeptide from an AAV serotype chosen from: AAV1, AAV2, AAV3, AAV4, AAV5, AAV6, AAV7, AAV8, AAV9, AAV10, AAV11, and AAV12. In some embodiments the viral vector comprises a polypeptide comprising VP1 from an AAV serotype chosen from: AAV1, AAV2, AAV3, AAV4, AAV5, AAV6, AAV7, AAV8, AAV9, AAV10, AAV11, and AAV12. In some embodiments the viral vector comprises a polypeptide comprising VP2 from an AAV serotype chosen from: AAV1, AAV2, AAV3, AAV4, AAV5, AAV6, AAV7, AAV8, AAV9, AAV10, AAV11, and AAV12. In some embodiments the viral vector comprises a polypeptide comprising VP3 from an AAV serotype chosen from: AAV1, AAV2, AAV3, AAV4, AAV5, AAV6, AAV7, AAV8, AAV9, AAV10, AAV11, and AAV12. In some embodiments, the viral vector comprises VP1, VP2 and VP3 polypeptides that is at least 60%, 65%, 70%, 75%, 80%, 85%, 90%, 95%, 96%, 97%, 98% or 99% identical over the VP1, VP2, and/or VP3 polypeptides from AAV6. In some embodiments, the viral vector comprises VP1, VP2 and VP3 polypeptides that is at least 60%, 65%, 70%, 75%, 80%, 85%, 90%, 95%, 96%, 97%, 98% or 99% identical over the VP1, VP2, and/or VP3 polypeptides from AAV7. In some embodiments, the viral vector comprises VP1, VP2 and VP3 polypeptides that is at least 60%, 65%, 70%, 75%, 80%, 85%, 90%, 95%, 96%, 97%, 98% or 99% identical over the VP1, VP2, and/or VP3 polypeptides from AAV8. In some embodiments, at least one, two or three VP polypeptides of the AAV capsid are mutated such that they are deficient in binding to heparan sulfate when exposed to a cell.

The term “effective amount” means that amount of compound, composition or agent that will elicit the biological response of a subject that is being sought. In some embodiments, the therapeutically effective amount is administered by a medical doctor or other clinician. The terms “specific binding” or “specifically binding”, as used herein, in reference to the interaction of an antibody, a protein, or a peptide with a second chemical species, mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

“Variant” used herein with respect to a nucleic acid means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of +2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within +2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

Nucleic acid molecules or nucleic acid sequences of the disclosure include those coding sequences comprising one or more of: any gene-editing enzymes disclosed herein or functional variants thereof that possess no less than 65, 70, 75, 80, 85, 90, 95, 96, 97, 98, or 99% sequence identity with the coding sequences of the gene editing enzymes disclosed herein.

“Vector” used herein means, in respect to a nucleic acid sequence, a nucleic acid sequence comprising a regulatory nucleic acid sequence that controls the replication of an expressible gene. A vector may be either a self-replicating, extrachromosomal vector or a vector which integrates into a host genome. Alternatively, a vector may also be a vehicle comprising the aforementioned nucleic acid sequence. A vector may be a plasmid, bacteriophage, viral particle (isolated, attenuated, recombinant, etc.). A vector may comprise a double-stranded or single-stranded DNA, RNA, or hybrid DNA/RNA sequence comprising double-stranded and/or single-stranded nucleotides. In some embodiments, the vector is a viral vector that comprises a nucleic acid sequence that is a viral packaging sequence responsible for packaging one or plurality of nucleic acid sequence that encode one or a plurality of polypeptides. In some embodiments, the vector comprises a viral particle comprising a nucleic acid sequence operably linked to a regulatory sequence, wherein the nucleic acid sequence encodes a fusion protein comprising one or a plurality of AAV VP polypeptides or fragments thereof.

“Viral vector” as disclosed herein means, in respect to a vehicle, any virus, virus-like particle, virion, viral particle, or pseudotyped virus that comprises a nucleic acid sequence that directs packaging of a nucleic acid sequence in the virus, virus-like particle, virion, viral particle, or pseudotyped virus. In some embodiments, the virus, virus-like particle, virion, viral particle, or pseudotyped virus is capable of transferring a vector (such as a nucleic acid vector) into and/or between host cells. In some embodiments, the virus, virus-like particle, virion, viral particle, or pseudotyped virus is capable of transferring a vector (such as a nucleic acid vector) into and/or between target cells, such as B cells of a subject. The chimeric vectors of the present disclosure exploit in vivo non-pathogenic infectivity of adenoassociated viruses and the permissive antigen receptors displayed on target immune cells to direct transfer of nucleic acids contained within the vector into endogenous DNA of the one or more target cells. In some embodiments, the vectors comprise a randomized or non-randomized DNA barcode and the necessary machinery to direct endogenous DNA gene-editing of B cells. The description of Retroviridae, Adenoviridae, and Parvoviridae (which include adeno-associated viruses) including genome organization and replication, is detailed in references known in the art, such as Fields Virology (Fields et al., eds.). In some embodiments, the disclosure provide viral vector comprising a barcode that is a randomized nucleic acid sequence. In some embodiments, the viral vector further comprises a nucleic acid sequence encoding a gene-editing enzyme; wherein the barcode is positioned between two gene-editing enzyme recognition sequences, such as transposons, such that, when the gene-editing enzyme is encoded by the nucleic acid sequence, the enzyme acts on the transposon sequences to excise the barcode out of the nucleic acid molecule and integrate the barcode into the genomic DNA of the cell.

A “viral particle” as that term is used herein, means a small particle from about ten nanometers to about one micrometer in diameter, comprising a structural viral protein (such as a viral core protein), around which one or a plurality of nucleic acid molecules are contained. In some embodiments, viral particles comprise a group of particles called lipoparticles which include enveloped virus-like particles. In some preferred embodiments, the lipoparticles are enveloped virus-like particles which comprise an enveloped viral core protein, a lipid bilayer, and one or more additional polypeptide on its surface. The viral particle may be from about 10 nm to about 500 nm in diameter, about 100 to about 500 nm, about 200 to about 400 nm, about to about 399 nm, about 500 nm to about 1000 nm, about 600 to about 900 nm, or about 700 to about 800 nm in diameter. In some embodiments, the viral particle does not encompass or comprise (is free of) cell membrane vesicles or cell lipids, which are typically produced using empirical methods and which are usually heterogeneous in size. In some embodiments, the lipoparticle also does not encompass liposomes, which typically lack core proteins that induce their formation. In some embodiments, the lipoparticle is dense, spherical, and/or homogeneous in size. In some embodiments, the viral particle is an AAV particle or lentiviral particle, such as HIV1 or a variant thereof.

A “time period sufficient to allow a viral particle to enter the cell” used as herein means any time period necessary for a viral particle to associate with and infect a cell after initial exposure to the cell. In some embodiments, the time period is about 30 seconds, about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, or about 30 minutes or more after exposure of the viral particle to a target cell.

A “time period sufficient to allow gene editing” in a cell used as herein means any time period necessary for a gene editing enzyme disclosed herein (e.g. a transposase) to associate with endogenous (or genomic) DNA of a target cell and edit the endogenous DNA after initial exposure to the cellular DNA. In some embodiments, the time period is about 30 seconds, about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, or about 30 minutes or more after exposure of the enzyme to the endogenous DNA of the target cell.

A “time period sufficient to allow clonal expansion” of a cell as used as herein means any time period necessary for a cell disclosed herein to divide carrying with it copies of its DNA and expand in culture after initial exposure to the labeling technique disclosed herein or after initial exposure to an antigen, such that, in the case of the latter, the cells immunoreact to the antigen and create a population of cells actively immunoreactive to the antigen. In some embodiments, the time period is about 30 minutes, about 1 hour, about 2 hours, about 3 hours, about 4 hours, about 5 hours, about 6 hours, about 7 hours, about 8 hours, about 9 hours, about hours, about 11 hours, about 12 hours, about 13 hours, about 14 hours, about 15 hours, about hours, about 17 hours, about 18 hours, about 19 hours, about 20 hours, about 21 hours, about hours, about 23 hours, about 24 hours, about 25 hours, about 26 hours, about 27 hours, about hours, about 29 hours, or about 30 hours or more after exposure of the cell to an antigen. In some embodiments, the time period sufficient to allow clonal expansion of the cell is from about hours to about 72 hours after initial exposure of the cell to the antigen. In some embodiments, the time period sufficient to allow clonal expansion of the cell is from about 1 day to about 30 days hours after initial exposure of the cell to the antigen.

### Compositions

The disclosure relates to viral particle-mediated entry of barcode DNA in immune cells for permanent labeling of the genomic DNA of the cell. Permanent labeling means that the genomic DNA of a cell becomes modified by the presence of a gene-editing enzyme recognizing sequences flanking a barcode, excising the barcode from the exogenous nucleic acid molecule and then editing the genomic DNA of the cell. Machinery for the gene-editing enzyme activity must also be transferred to the target cell.

The disclosure therefore relates to a nucleic acid molecule comprising a barcode flanked on the 5′ and 3′ side by one or more gene-editing recognition sites, and optionally, in a second nucleic acid molecule or positioned on the same nucleic acid molecule, a nucleic acid sequence encoding a gene-editing enzyme. The disclosure relates to a viral particle comprising the nucleic acid molecule or molecules disclosed herein, wherein the viral particle comprises a polypeptide capsid protein defining an outer structure of the particle and a polypeptide or polypeptides that make up a target protein complex. In some embodiments, the target protein complex comprises one or multiple polypeptides with one domain that incorporates within the outer protein shell of the particle, associates with the capsid proteins, or noncovalently associates with the capsid proteins (effectively physically connecting the complex to the viral particle by covalent or non-covalent binding); and another domain that comprises one or more antigen sequences. In some embodiments, the viral particle is deficient in infection, by, as a non-limiting example using AAV, having mutations on its VP1, VP2 and VP3 polypeptide sequences that confer an inability of the viral particle to infect a cell through heparan sulfate appearing on the surface of the cell. Instead, in some embodiments, the viral particles mediate entry into a target cell by the presence of the target protein complex through which an antigen or antigen fragment thereof binds to the immune machinery displayed on the surface of a target cell. After entry into the cell, the exogenous nucleic acid molecule comprising the barcode is edited by the presence of a gene-editing enzyme, resulting in integration of the barcode into the genomic DNA of the target cell. In some embodiments, the target cell is a B cell. In other embodiments, the target cell is primed or exposed to an antigen prior to entry by the virus resulting in antigen-specific activation of the target cell. In some embodiments, the B cell is an antigen-activated B cell.

In some embodiments, the compositions of the disclosure relate to a plurality of nucleic acid molecules, wherein at least one or a plurality of nucleic acid molecules individually or collectively comprise:
(i) a nucleic acid sequence encoding a gene editing enzyme or functional variant thereof; (ii) a first gene editing enzyme cleavage sequence; (iii) a barcode sequence; (iv) a second gene editing enzyme cleavage sequence.

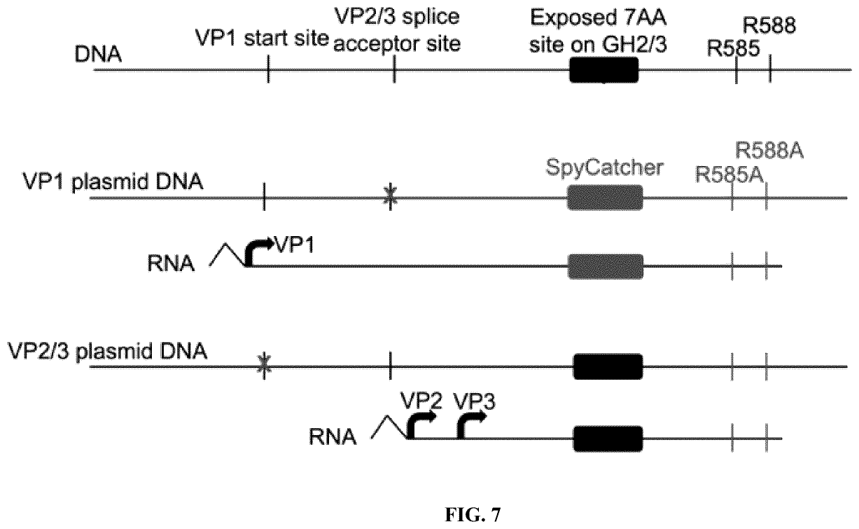

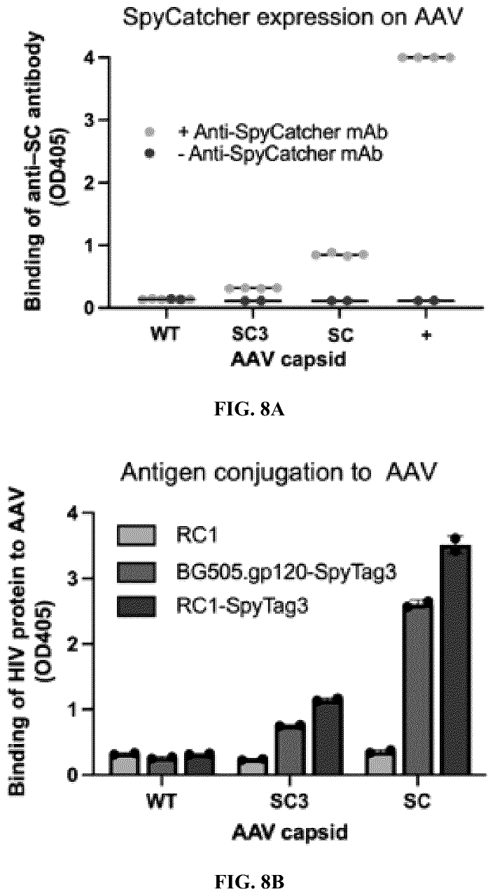

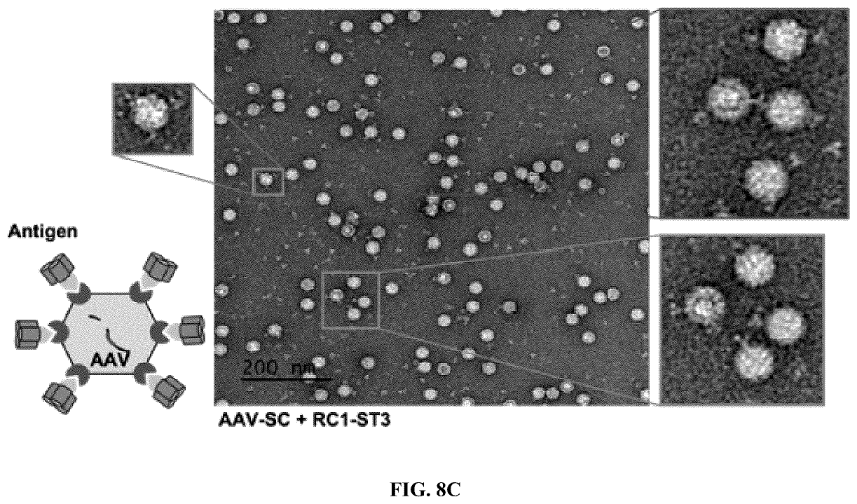

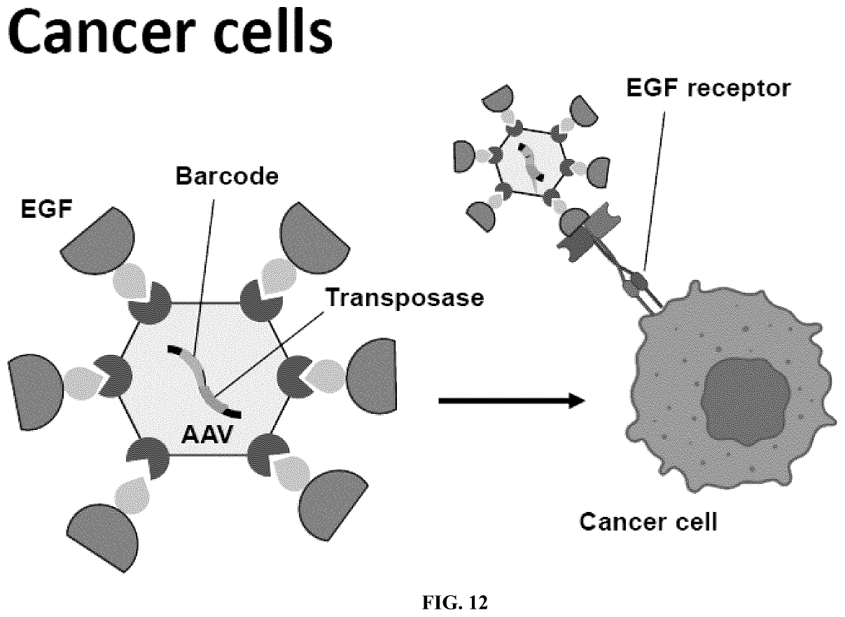

If such a plurality of nucleic acid molecules are used in manufacturing a viral particle, a single nucleic acid molecule may comprise (i) a first gene editing enzyme cleavage sequence; (ii) a barcode sequence; (iii) a second gene editing enzyme cleavage sequence, wherein the barcode sequence is positioned between the first and second gene editing enzyme cleavage sequences and those sequences (i), (ii), and (iii) are positioned between a viral ITR. Upon the presence of other individual or plurality of nucleic acid molecules comprising nucleic acid sequences encoding viral capsid proteins and other structural proteins required for viral assembly, a virus can be made encapsulating the nucleic acid molecule comprising (i), (ii) and (iii). If such a plurality of nucleic acid molecules are being used to edit a cell, the plurality of nucleic acid molecules may comprise a first nucleic acid molecule comprising: (i) a first gene editing enzyme cleavage sequence; (ii) a barcode sequence; (iii) a second gene editing enzyme cleavage sequence, wherein the barcode sequence is positioned between the first and second gene editing enzyme cleavage sequences; and a second nucleic acid molecule comprising a nucleic acid sequence encoding a gene editing enzyme or functional variant thereof; such that the cell encodes the gene editing enzyme or functional fragment thereof and another nucleic acid sequence carrying the barcode may be excised for gene editing. Compositions comprising: a viral particle comprising the barcode; and a second nucleic acid molecule comprising the sequence encoding the gene editing enzyme are contemplated by the disclosure. Compositions are also contemplated by the disclosure that relate to a first nucleic acid molecule encoding (i), (ii), (iii) above and a second nucleic acid molecule, such that both nucleic acid molecules (independent of a virus) may be transfected into a cell such that the cell expresses the gene editing enzyme and the gene editing enzyme acts on the barcode and destination of the barcode in the genomic DNA of the cell. Compositions of the disclosure also encompass those that comprise one, two, three or all four nucleic acid molecules set forth in FIG. 7. In such embodiments, all four of the nucleic acid molecules may be transfected into a cell to accomplish viral assembly of an AAV vector with a modified capsid comprising a chimeric protein or fusion protein with an antigen domain, such that the antigen may facilitate entry into the cell by presentation of the antigen on a viral particle. In some embodiments, the target cells may be targeted by the natural protein-protein interaction of the antigen on the viral particle and the antigen ligand naturally expressed on the surface of the cell. In some embodiments, the composition comprises a cell such as a cancer cell of FIG. 12 that expressed EGFR or a functional variant thereof and a viral particle displaying EGF or a functional variant thereof. In those embodiments, the cancer cell may be permanently tagged.

### Viral Particles

Viral vectors for use in the present methods and compositions include recombinant retroviruses, adenovirus, adeno-associated virus, alphavirus, and lentivirus, comprising the targeting peptides described herein and optionally a transgene for expression in a target tissue. In some embodiments, the viral vectors or particles are non-infectious. In some embodiments, the vector particles are attenuated. In some embodiments, the viral particles comprise one, two, three or more capsid proteins that are modified to remove residues responsible for binding to heparan sulfate and thereby reduce delivery of the virus into a cell by used of their endogenous polypeptide machinery. Instead, some embodiments of the disclosure relate to a viral particle comprising a targeting domain, the targeting domain comprising an antigen amino acid sequence and one domain mediating association of the antigen amino acid sequence to the viral particle. In some embodiments, the viral particle comprises a chimeric VP molecule displaying antigen on the viral particle surface, such antigen mediating association with one or more cells comprising a ligand for antigen binding.

The present disclosure provides, among other things, compositions that can be used, for example, to selectively enter an immune cell expressing specific cell surface markers that are ligands to antigens displayed on the surface of the viral particles. By utilizing viral particles to transduce the same cell type, the viral particles comprising barcodes can tag cells or label cells such that antigen-specific immune molecules such as antibodies and antibody fragments can be tracked or assigned to certain population of immune cells comprising barcodes disclosed herein.

The present disclosure also provides, among other things, compositions that can be used, for example, to selectively activate a cell expressing a ligand specific for the antigen and then track the immunoactivity of a single cell by subsequently isolating the cell, sequencing the barcode and correlating the barcode to the immunoactivity of the cell.

In some embodiments, the viral particle encodes or displays a polypeptide of interest, such as an antigen, which can also be referred to as a molecule of interest. In some embodiments, if, comprising a target protein domain, the target protein domain comprises a domain that mediates association or binding to one or more viral capsid proteins.

In some embodiments, a composition comprising an engineered viral particle comprising an engineered envelope harboring a glycoprotein, or a variant thereof, a chimeric gag protein, and an engineered targeting moiety for binding to a target cell; and a nucleic acid encoding a polypeptide of interest is provided.

In some embodiments, an engineered viral particle comprising an engineered envelope comprising a lentivirus glycoprotein, or a variant thereof, a gag-pol protein, and an engineered targeting moiety for binding to a target cell; and a nucleic acid encoding a polypeptide of interest is provided. In some embodiments, the antigen is fused to a lentiviral glycoprotein. In some embodiments, the viral particle is a replication deficient lentivirus.

Some embodiments useful for delivery of nucleic acids in the present methods is the adeno-associated virus (AAV). AAV is a tiny non-enveloped virus having a 25 nm capsid. No disease is known or has been shown to be associated with the wild type virus. AAV has a single-stranded DNA (ssDNA) genome. AAV has been shown to exhibit long-term episomal transgene expression, and AAV has demonstrated excellent transgene expression in the brain, particularly in neurons. Vectors containing as little as 300 base pairs of AAV can be packaged and can integrate. Space for exogenous DNA is limited to about 4.7 kb. An AAV vector such as that described in Tratschin et al., Mol. Cell. Biol. 5:3251-3260 (1985) can be used to introduce DNA into cells. A variety of nucleic acids have been introduced into different cell types using AAV vectors (see for example Hermonat et al., Proc. Natl. Acad. Sci. USA 81:6466-6470 (1984); Tratschin et al., Mol. Cell. Biol. 4:2072-2081 (1985); Wondisford et al., Mol. Endocrinol. 2:32-39 (1988); Tratschin et al., J. Virol. 51:611-619 (1984); and Flotte et al., J. Biol. Chem. 268:3781-3790 (1993). There are numerous alternative AAV variants (over 100 have been cloned), and AAV variants have been identified based on desirable characteristics. In some embodiments, the AAV is AAV1, AAV2, AAV4, AAV5, AAV6, AV6.2, AAV7, AAV8, AAV9, rh. 10, rh.39, rh.43 or CSp3; for CNS use, in some embodiments the AAV is AAV1, AAV2, AAV4, AAV5, AAV6, AAV8, or AAV9. As one example, AAV9 has been shown to somewhat efficiently cross the blood-brain barrier. Using the present methods, the AAV capsid can be genetically engineered to increase permeation across the BBB, or into a specific tissue, by insertion of a targeting sequence as described herein into the capsid protein, e.g., into the AAV9 capsid protein VP1 between amino acids 588 and 589.

Modifications of the VP polypeptide for conferring deficiency in heparan sulfate binding are underlined in the amino acid sequence as follows:

MAADGYLPDWLEDTLSEGIRQWWKLKPGPPPPKPAERHKDDSRGLVLPGY
KYLGPFNGLDKGEPVNEADAAALEHDKAYDRQLDSGDNPYLKYNHADAEF
QERLKEDTSFGGNLGRAVFQAKKRVLEPLGLVEEPVKTAPGKKRPVEHSP
VEPDSSSGTGKAGQQPARKRLNFGQTGDADSVPDPQPLGQPPAAPSGLGT
NTMATGSGAPMADNNEGADGVGNSSGNWHCDSTWMGDRVITTSTRTWALP
TYNNHLYKQISSQSGASNDNHYFGYSTPWGYFDFNRFHCHFSPRDWQRLI
NNNWGFRPKRLNFKLFNIQVKEVTQNDGTTTIANNLTSTVQVFTDSEYQL
PYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLSRTNT
PSGTTTQSRLQFSQAGASDIRDQSRNWLPGPCYRQQRVSKTSADNNNSEY
SWTGATKYHLNGRDSLVNPGPAMASHKDDEEKFFPQSGVLIFGKQGSEKT
NVDIEKVMITDEEEIRTTNPVATEQYGSVSTNLQAGNAQAATADVNTQGV
LPGMVWQDRDVYLQGPIWAKIPHTDGHFHPSPLMGGFGLKHPPPQILIKN
TPVPANPSTTFSAAKFASFITQYSTGQVSVEIEWELQKENSKRWNPEIQY
TSNYNKSVNVDFTVDINGVYSEPRPIGTRYLTRNL

To confer specificity of the viral particles to target cells, viral particles can be pseudotyped. Capsid proteins and envelope glycoproteins are implicated in virus attachment and interactions with cellular receptors, determining cell tropism. Manipulation of these viral surface proteins therefore may improve the transduction capacity of these vectors through an antigen or fragment thereof, expanding their tropism for immune cells and restricting their tropism through ordinary pathogenic pathways.

In some embodiments, a virus particle comprising a protein target domain comprising an antigen or fragment thereof that binds to a target on a cell. Nucleic acid molecules that encode a polypeptide of interest and package the protein into the viral particle are provided by the disclosure.

The protein targeting domain can comprise any antigen domain or cell targeting domain, or, alternatively, a plurality of polypeptides that are chimerically presented on the surface of the viral particle. In some embodiments, the polypeptide comprise a domain comprising an antigen and a domain comprising one or a combination of: an scFv, an antigen binding domain, a VHH, a DARPin, an adnectin, an affibody, an affilin, an affimer, an affitin, an alphabody, an anticalin, an aptamer, an armadillo repeat protein-based scaffold, an atrimer, an avimer, a fynomer, a knottin, a kunitz domain peptide, a monobody, a nanofitin, or any combination thereof, a Centryn, Stem Cell Factor protein (SCF, KIT-ligand, KL, or steel factor) or a moiety that binds to cKit (CD117), CD4, CD8, CD3, CD5, CD6, CD7, CD2, BCR, TCR alpha, TCR beta, TCR gamma, TCR delta, CD10, CD34, CD14, CD68, CCR7, CD62L, CD25, CCR2, CCR3, CCR4, CCR5, CCR6, CCR7, CXCR3, CD39, CD73, CTLA-4, GITR, LAG-3, LRRC32, Neurophili-1, and CX3CR1. In some embodiments, the target is cKit (CD117), CD4, CD8, CD3, CD5, CD6, CD7, CD2, TCR alpha, TCR beta, TCR gamma, TCR delta, CD10, CD34, CD14, CD68, CCR7, CD62L, CD25, CCR2, CCR3, CCR4, CCR5, CCR6, CCR7, CXCR3, CD39, CD73, CTLA-4, GITR, LAG-3, LRRC32, Neurophili-1, and CX3CR1.

In some embodiments, the targeting moiety that binds to a target, an antibody, an scFv, an antigen binding domain, a VHH, a DARPin, an adnectin, an affibody, an affilin, an affimer, an affitin, an alphabody, an anticalin, an aptamer, an armadillo repeat protein-based scaffold, an atrimer, an avimer, a fynomer, a knottin, a kunitz domain peptide, a monobody, a nanobody, a nanofitin, or any combination thereof. In some embodiments, the targeting moiety is Stem Cell Factor protein (SCF, KIT-ligand, KL, or steel factor) or a moiety that binds to cKit (CD117), (SCF, KIT-ligand, KL, or steel factor) or a moiety that binds to cKit (CD117), CD4, CD8, CD3, CD5, CD6, CD7, CD2, TCR alpha, TCR beta, TCR gamma, TCR delta, CD10, CD34, CD14, CD68, CCR7, CD62L, CD25, CCR2, CCR3, CCR4, CCR5, CCR6, CCR7, CXCR3, CD39, CD73, CTLA-4, GITR, LAG-3, LRRC32, Neurophili-1, and CX3CR1. In some embodiments, the protein targeting domain comprises a modified viral capsid protein that comprises SpyCathcer, SpyTag and an antigen. SpyCatcher is encoded by SEQ ID NO:4:

[SEQ ID NO: 5]
atgtcgtactaccatcaccatcaccatcacgattacgacatcccaacga
ccgaaaacctgtattttcagggcgccatggttgataccttatcaggttt
atcaagtgagcaaggtcagtccggtgatatgacaattgaagaagatagt
gctacccatattaaattctcaaaacgtgatgaggacggcaaagagttag
ctggtgcaactatggagttgcgtgattcatctggtaaaactattagtac
atggatttcagatggacaagtgaaagatttctacctgtatccaggaaaa
tatacatttgtcgaaaccgcagcaccagacggttatgaggtagcaactg
ctattacctttacagttaatgagcaaggtcaggttactgtaaatggcaa
agcaactaaaggtgacgctcatatttaa, or a functional
variant thereof and the ligand is SpyTag:
AHIVMVDAYKPTK

In some embodiments, the viral particle displays a polypeptide comprising at least about 75%, 80%, 85%, 90%, 91%, 92%, 93%, 94%, 95%, 96%, 97%, 98%, or 99% sequence identity to the SpyCatcher sequence. In some embodiments, the viral particle displays a polypeptide comprising at least about 75%, 80%, 85%, 90%, 91%, 92%, 93%, 94%, 95%, 96%, 97%, 98%, or 99% sequence identity to the SpyTag sequence. The disclosure also relates to an AAV viral particle comprising a modified VP1, VP2 and VP3 polypeptides, the VP1, VP2 and VP3 polypeptides comprising one or more mutations that render the viral particle replication and infection-deficient. In some embodiments, the modified VP polypeptides confer an inability of the viral particle to secure entry into a cell through heparan sulfate.

The disclosure also relates to an AAV viral particle comprising a modified VP1, VP2 and VP3 polypeptides, the VP1, VP2 and VP3 polypeptides comprising one or more mutations that render the viral particle replication and infection-deficient; and wherein the viral particle comprises a polypeptide that is a target protein binding domain, comprising a chimeric polypeptide, wherein the chimeric polypeptide comprises an AAV viral sequence, a chimeric polypetide first domain comprising SpyCatcher or a functional variant thereof, a SpyTag or a functional variant thereof and antigen; wherein the antigen is displayed on the portion of the polypeptide most distal from the viral surface. In some embodiments, the viral particle further comprises a nucleic acid molecule disclosed herein. In some embodiments, the nucleic acid molecule comprises:
a bar code domain; and a first gene editing enzyme cleavage sequence or recognition sequence and a second gene editing enzyme cleavage or recognition sequence; wherein the first gene editing enzyme cleavage or recognition sequence is positioned within about 20 nucleotides upstream from the barcode domain; and wherein the second gene editing enzyme cleavage or recognition sequence is positioned within about 20 nucleotides downstream from the 3′ end of the barcode domain. In some embodiments, the nucleic acid molecule comprises a nucleic acid sequence encoding a gene editing enzyme or functional variant thereof; wherein the nucleic acid sequence encoding the gene editing enzyme or functional variant thereof is operably linked to a regulatory sequence. In some embodiments, the nucleic acid sequence encoding the gene editing enzyme or functional variant thereof is positioned between the first and second gene editing enzyme cleavage or recognition sequence.

Chimeric and fusion proteins of the disclosure can be produced by standard recombinant DNA techniques. In some embodiments, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, e.g., Ausubel et al., supra). Moreover, many expression vectors are commercially available that already encode a fusion moiety (e.g., a GST polypeptide). A nucleic acid encoding a polypeptide of the disclosure can be cloned into such an expression vector such that the fusion moiety is linked in frame to the polypeptide of the disclosure. In some embodiments, the disclosed method comprise a method of gene editing wherein a chimeric fusion protein comprising a probe is exposed to any resulting protein targeting domain or complex, such that interaction of the probe with the domain or complex results in a non-covalent or covalent binding event between the probe and amplicon, and in such as case, the method comprises a step wherein the probe is identified or quantified by excitation of the probe to a stimulus or exposure of the probe to a chemiluminescent agent. In such embodiments, the excitation or exposure emits a detectable signal which can be quantified by known methods in the art such as quantitative fluorescence, semi-quantitative fluorescence of a Western blot exposed to an antibody or antibody fragment specific for the probe.

The disclosure provides a nucleic acid molecule, wherein the nucleic acid molecule comprises an engineered AAV genome comprising:
i.a barcode DNA;
ii.a nucleic acid sequence encoding a gene editing enzyme or functional variant thereof; wherein the nucleic acid sequence encoding the gene editing enzyme or functional variant thereof is operably linked to a regulatory sequence; and
iii.a first gene editing enzyme cleavage sequence and a second gene editing enzyme cleavage sequence; wherein the first gene editing enzyme cleavage sequence is positioned within about 20 nucleotides upstream from the 5′ end of barcode; and wherein the second gene editing enzyme cleavage sequence is positioned within about nucleotides downstream from the 3′ end of the barcode DNA.
   In some embodiments, viral particles of the disclosure comprise infection-deficient VP polypeptides and associate with a nanobody specific for a viral antigen, such as gp120, gp40, SARS spike protein. In some embodiments, viral particles of the disclosure comprise infection-deficient, chimeric VP polypeptides wherein one domain of the chimeric polypeptide is a nanobody specific for a viral antigen or cancer antigen. In such embodiments the viral antigen or the cancer antigen mediates viral entry by association and internalization of an antigen complex on the surface of a target cell comprising a ligand for the antigen. FIGs10, 11 and 12 exemplify antigen-ligand pairs. In some embodiments the viral particle is an AAV particle comprising a infection deficient VP polypeptide and an MHC II protein and PADRE. The viral particles disclosed herein may target cells expressing a T cell receptor or a B cell receptor.

### Barcodes

Embodiments of the disclosure relate to nucleic acid molecules that comprise a barcode sequence. Barcode sequences may be any one or more DNA sequences, RNA sequences or hybrid DNA/RNA sequences. Barcode sequences may be randomized or specific including one or more known primer sequences that, upon exposure to a complementary nucleic acid sequence under proper conditions can result in amplification and/or sequencing of the barcode. Barcode sequences may be generated by known methods. For example, generation of libraries of barcodes are disclosed, for example, in Scientific Reports, volume 7, Article number: 13899 (2017), which is incorporated by reference in its entirety. Briefly, barcodes may comprise three components: a batch code of length l_{b} bp, a linker of length 2 bp, and a target code of length lₜ. The term “batch code” may refer collectively to a batch code that is appended with a linker. Thus barcode length may be decomposed as L=l_{b}+lₜ+2. Barcode structure interpretation differs depending on the experimental context. In the use case when all N barcodes in a library are intended for use in a single experiment, each barcode serves to identify a unique target, so that the batch/target interpretation ceases to apply in any meaningful sense. In other words, each barcode functions as a “target code” in its own right. However, in a second use case the batch/target code construct becomes important in the context of pooled experiments where the batch code is used to identify the group to which the target molecule belongs. In this case the batch code functions as a unique identifier of an experiment batch, while a target code serves to identify a unique target within a particular batch. Examples of such applications include competition experiments and Barcode Fusion Genetics.

In general, a barcode library may be generated by nucleic acid synthesis or purchased from a commercial vendor. If generated by synthesis, the library is composed of n b linked batch codes and nₜ target codes. The N=n_{b}×nₜ barcodes in the library are made up from all pairwise concatenations of the linked batch codes with the target codes. Therefore, in a pooled experimental setting, n_{b} corresponds to the maximum number of different experiments, and nₜ the maximum number of targets in a particular experiment. The algorithm requires input parameters N (number of barcodes), n_{b} (number of batch codes), nₜ (number of target codes), L (barcode length), l_{b} (batch code length), lₜ (target code length), and generation constraints d (minimum Hamming distance), m (maximum homopolymer length), GC-content min/max bounds, and a blacklist of proscribed sequences.

Barcode library generation may be broken down into two phases: first, a set of n_{b} batch codes is produced, and second, a set of nₜ target codes are generated in such a manner that each target code when postfixed to any linked batch code passes all the filters. It is this last step of ensuring that all the filters are passed that makes for the computationally intensive part of the generation process. Individual cell types (e.g., such as cell lines or immune cells) can be labeled with any identifying nucleic acid tag, e.g., DNA barcode, and any combination of cell lines may be mixed together for assays so long as they are capable of growth in the same conditions allows optimization for different applications. A number of nucleic acid tags are known in the art; in preferred embodiments the nucleic acid tag comprises a core of a sufficient number of nucleotides to provide specificity, e.g., 20-26 nucleotides, e.g., 22-24 nucleotides, and is designed to be both unique to each cell type, readily amplifiable (e.g., lacking in substantial predicted secondary structures such as hairpins), and not readily cross-hybridizable, to give results that can be specifically interpreted with confidence. In some embodiments, the nucleic acid tags further comprise flanking sequences that allow binding of a set of primers for amplifying the variable, unique core sequence; in some embodiments, the flanking sequences are all the same in all of the cells of the plurality of genetically heterogeneous cell types (though the core sequences vary from cell type to cell type as described herein).

Here we describe a Markov chain model of order m for nucleotide sequence generation. The model is used to stochastically generate nucleotide sequences of prescribed length with maximum homopolymer length m. This model is important DNA barcode generation framework. In practice, the use of this model for nucleotide sequence generation increases the number of candidate barcodes that pass the homopolymer filter, compared to what happens when sequences are generated at random.

Let S={A, C, G, T} be the set of nucleotide bases. A nucleotide sequence of length n is defined as x₁ₙ=x₁x₂, . . . , xₙ for n realizations of xₖ∈S for 1≤k≤n where xₖxₖ₊₁ denotes concatenation. Markov chain models of order m is defined by the transition rule and energy, but it did not turn out to perform significantly better than our simpler model.

Any sequences may be designed for a barcode. Exemplified barcodes sequences follow:

Barcode ID: Seq (5′−>3′)
BC1
AGCTTGACCGGTACAGTACGCTCAGTTCTATCTGACCTATATGAGACGT
CAACTTTTTCAAGAGTGCCACGCCCGAAGGTCACGTATAGGGCAGAACT
ATATCTGTCAAAGATGACGGTACCGACACGATATTTGCTGAAGTCCATT
CCGAAGGTGATGTACGTCGATTCTTATGGAATTAATCTCGTCGCGCGAT
GTTGTGCCACAACGTCTATATGATGGCGTACGTAAAGG
BC2
AGCTTGACCGGTACAGTAAGAGCACCAGTGCCTTGATTCTACTCAGTCT
CAACTTTTTCAAGAGTGCCACGCCCGAAGGTCACGTATAGGGCAGAACT
ATATCTGTCAAAGATGACGGTACCGACACGATATTTGCTGAAGTCCATT
CCGAAGGTGATGTACGTCGATTTCGTCTGACTGAACATACGGCCTATGA
CTCGTGCCACAACGTCTATATGATGGCGTACGTAAAGG
BC3
AGCTTGACCGGTACAGTATAATGGCAAGGTGCCGCTTCCGGCAATGGAA
CAACTTTTTCAAGAGTGCCACGCCCGAAGGTCACGTATAGGGCAGAACT
ATATCTGTCACTAGAGATGACGGTACCGACACGATATTTGCTGAAGTCC
ATTCCGAAGGTGATGTACGTCGATTGCCGTAACCGAACCATTCTCGGTT
GCCTCTGTGCCACAACGTCTATATGATGGCGTACGTAAAGG
BC4
AGCTTGACCGGTACAGTACTAATGATGGTCGGCCTATCAGTCAACCATT
CAACTTTTTCAAGAGTGCCACGCCCGAAGGTCACGTATAGGGCAGAACT
ATATCTGTCAAAGATGACGGTACCGACACGATATTTGCTGAAGTCCATT
CCGAAGGTGATGTACGTCGATTGAGCGCAATAAACAAGGCGTGTATGTA
GAAGTGCCACAACGTCTATATGATGGCGTACGTAAAGG
BC5
AGCTTGACCGGTACAGTATTCTATGGTTCCTCGCAACCTGGATGCTTAT
CAACTTTTTCAAGAGTGCCACGCCCGAAGGTCACGTATAGGGCAGAACT
ATATCTGTCAAAGATGACGGTACCGACACGATATTTGCTGAAGTCCATT
CCGAAGGTGATGTACGTCGATTATGTCGTGGTAGAGTGCGGCTGCCTGG
TGGGTGCCACAACGTCTATATGATGGCGTACGTAAAGG

Gene Editing Enzymes Gene-editing enzymes are provided by the disclosure as well as nucleic acid sequences encoding the same. Any gene-editing enzyme is contemplated by the disclosure including meganucleases, transposases or Cas proteins. It should be understood that the enzymes with mutations (such as the Cas9 enzyme) or any functional variants thereof described herein are intended to include amino acid sequences comprising polypeptides bearing one or more insertions, deletions, or substitutions, or any combination thereof, of amino acid residues as well as modifications other than insertions, deletions, or substitutions of amino acid residues, such as but not limited to conservative amino acid substitutions.

CRISPR enzyme (or “Cas protein”) or a nucleotide sequence encoding one or more Cas proteins. Any protein capable of enzymatic activity in cooperation with a guide sequence is a Cas protein. In some embodiments, the disclosure relates to a system comprises a vector comprising a regulatory element operably linked to an enzyme-coding sequence encoding a CRISPR enzyme, such as a Cas protein from the Cas family of enzymes. In some embodiments, the disclosure relates to a system, composition, or pharmaceutical composition comprising any one or plurality of Cas proteins either individually or in combination with one or a plurality of guide sequences. Compositions of one or a plurality of Cas proteins may be administered to a subject with any of the disclosed guide sequences sequentially or contemporaneously. Non-limiting examples of Cas proteins include Cas1, Cas1B, Cas2, Cas3, Cas4, Cas5, Cas6, Cas7, Cas8, Cas9 (also known as Csn1 and Csx12), Cas1O, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx1O, Csx16, CsaX, Csx3, Csx1, Csx15, Csf1, Csf2, Csf3, Csf4, type V CRISPR-Cas systems, variants and fragments thereof, or modified versions thereof comprising at least 70% sequence identity to to the sequences of Table E. These enzymes are known; for example, the amino acid sequence of S. pyogenes Cas9 protein may be found in the SwissProt database under accession number Q99ZW2. In some embodiments, the unmodified CRISPR enzyme has DNA cleavage activity, such as Cas9. In some embodiments the CRISPR enzyme is Cas9, and may be Cas9 from S. pyogenes or S. pneumoniae. In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence. In some embodiments, a vector encodes a CRISPR enzyme or Cas protein that is mutated to with respect to a corresponding wild-type enzyme, such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A. In some embodiments, a Cas9 nickase may be used in combination with guide sequenc(es), e.g., two guide sequences, which target respectively sense and antisense strands of the DNA target. This combination allows both strands to be nicked and used to induce NHEJ.

As a further example, two or more catalytic domains of Cas9 (RuvC I, RuvC II, and RuvC III) may be mutated to produce a mutated Cas9 substantially lacking all DNA cleavage activity. In some embodiments, a D10A mutation is combined with one or more of H840A, N854A, or N863A mutations to produce a Cas9 enzyme substantially lacking all DNA cleavage activity. In some embodiments, a CRISPR enzyme is considered to substantially lack all DNA cleavage activity when the DNA cleavage activity of the mutated enzyme is less than about 25%, 10%, 5%, 1%, 0.1%, 0.01%, or lower with respect to its non-mutated form. Other mutations may be useful; where the Cas9 or other CRISPR enzyme is from a species other than S. pyogenes, mutations in corresponding amino acids may be made to achieve similar effects.

TABLE D

Cas proteins
Accession Numbers of Cas proteins (or those
related with Cas-like function) and Nucleic
Acids encoding the same. All amino acid and nucleic
acid sequences associated with the Accession
Numbers below as of Sep. 28, 2023, are incorporated
by reference in their entireties. Any mutants or
variants that comprise at least about 70, 75, 80, 85, 90,
95, 96, 97, 98, 99% sequence identity to the
encoded nucleic acids or amino acids set forth in the
Accession Numbers below are also incorporated by
reference in their entireties. NC_014644.1; NC_002967.9;
NC_007929.1; NC_000913.3; NC_004547.2;
NC_009380.1; NC_011661.1; NC_010175.1;
NC_010175.1; NC_010175.1; NC_003413.1;
NC_000917.1; NC_002939.5; NC_018227.2;
NC_004829.2; NC_021921.1; NC_014160.1;
NC_011766.1; NC_007681.1; NC_021592.1;
NC_021592.1; NC_021169.1; NC_020517.1;
NC_018656.1; NC_018015.1; NC_018015.1;
NC_017946.1; NC_017576.1; NC_017576.1;
NC_015865.1; NC_015865.1; NC_015680.1;
NC_015680.1; NC_015474.1; NC_015435.1;
NC_013790.1; NC_013790.1; NC_012883.1;
NC_012470.1; NC_016051.1; NC_010610.1;
NC_009515.1; NC_008942.1; NC_007181.1;
NC_007181.1; NC_006624.1; NC_006448.1;
NC_002935.2; NC_002935.2; NC_002950.2;
NC_002950.2; NC_002663.1; NC_002663.1;
NC_004557.1; NC_004557.1; NC_019943.1;
NC_019943.1; NC_019943.1; NC_017459.1;
NC_017459.1; NC_015518.1; NC_015460.1;
NC_015416.1; NC_014933.1; NC_013961.1;
NC_013202.1; NC_013158.1; NC_009464.1;
NC_008508.1; NC_007426.1; NC_000917.1;
NC_003901.1; NC_003901.1; NC_003106.2;
NC_009434.1; NC_005085.1; NC_005085.1;
NC_020247.1; NC_020247.1; NC_020246.1;
NC_020246.1; NC_018224.1; NC_015943.1;
NC_011138.3; NC_009778.1; NC_006834.1;
NC_014228.1; NC_010002.1; NC_013892.1;
NC_010296.1; NC_009615.1; NC_012632.1;
NC_012632.1; NC_012588.1; NC_012588.1;
NC_007643.1; NC_002939.5; NC_011296.1;
NC_011296.1; NC_018609.1; NC_021355.1;
NC_021355.1; NC_020800.1; NC_019942.1;
NC_019792.1; NC_015958.1; NC_015678.1;
NC_015636.1; NC_015562.1; NC_014222.1;
NC_014222.1; NC_014002.1; NC_013887.1;
NC_013156.1; NC_011832.1; NC_009953.1;
NC_009635.1; NC_009634.1; NC_008618.1;
NC_007955.1; NC_007955.1; NC_007955.1;
NC_007955.1; NC_007955.1; NC_007796.1;
NC_002754.1; NC_002754.1; NC_011835.1;
NC_013198.1; NC_000962.3; NC_002163.1;
NC_017034.1; NC_009089.1; NC_008698.1;
NC_020419.1; NC_020419.1; NC_020419.1;
NC_015847.1; NC_014374.1; NC_013520.1;
NC_010482.1; NC_009776.1; NC_009776.1;
NC_009033.1; NC_000916.1; NC_018015.1;
NC_015518.1; NC_014537.1; NC_009440.1;
NC_007644.1; NC_007644.1; NC_022246.1;
NC_019943.1; NC_016023.1; NC_016023.1;
NC_015416.1; NC_013722.1; NC_013722.1;
NC_009464.1; NC_007643.1; NC_007643.1;
NC_007643.1; NC_003106.2; NC_004342.2;
NC_018658.1; NC_017276.1; NC_017275.1;
NC_016112.1; NC_016112.1; NC_003552.1;
NC_003197.1; NC_003198.1; NC_012726.1;
NC_012623.1; NC_015964.1; NC_023069.1;
NC_023044.1; NC_022777.1; NC_022777.1;
NC_022777.1; NC_013769.1; NC_013769.1;
NC_011832.1; NC_011296.1; NC_009712.1;
NC_009634.1; NC_009439.1; NC_009135.1;
NC_008599.1; NC_007796.1; NC_007796.1;
NC_007796.1; NC_007355.1; NC_021082.1;
NC_018001.1; NC_009785.1; NC_022084.1;
NC_018092.1; NC_014804.1; NC_014147.1;
NC_009053.1; NC_000961.1; NC_000961.1;
NC_021058.1; NC_018876.1; NC_018876.1;
NC_018081.1; NC_011567.1; NC_016901.1;
NC_014500.1; NC_013715.1; NC_019977.1;
NC_019042.1; NC_017274.1; NC_015954.1;
NC_015676.1; NC_015320.1; NC_014122.1;
NC_014122.1; NC_013407.1; NC_014961.1;
NC_013926.1; NC_013926.1; NC_021353.1;
NC_008818.1; NC_021058.1; NC_015151.1;
NC_013849.1; NC_009051.1; NC_018876.1;
NC_018876.1; NC_014507.1; NC_015574.1;
NC_014500.1; NC_012622.1; NC_012589.1;
NC_009515.1; NC_017275.1; NC_000913.3;
NC_017527.1; NC_018227.2; NC_007355.1;
NC_014106.1; NC_010610.1; NC_008054.1;
NC_007164.1; NC_015760.1; NC_009953.1;
NC_010572.1; NC_009613.3; NC_014334.1;
NC_008526.1; NC_026150.1; NC_015776.1;
NC_007116.6; NC_012779.2; NC_003901.1;
NC_020892.1; NC_011832.1; NC_003143.1;
NC_003143.1; NC_008800.1; NC_011308.1;
NC_008942.1; NC_007297.1; NC_005877.1;
NC_005877.1; NC_002689.2; NC_006085.1;
NC_004116.1; NC_010397.1; NC_009917.1;
NC_012490.1; NC_006067.1; NW_004197518.1;
NC_022777.1; NC_019042.1; NC_004547.2;
NC_002695.1; NC_017634.1; NC_003143.1;
NC_002737.2; NC_002737.2; NC_000918.1;
NC_020913.1; NC_006448.1; NC_022093.1;
NC_022093.1; NC_015680.1; NC_007297.1;
NC_004350.2; NC_004350.2; NC_004350.2;
NC_004350.2; NC_003454.1; NC_000853.1;
NC_018876.1; NC_009440.1; NC_009009.1;
NC_009009.1; NC_002932.3; NC_002932.3;
NC_026150.1; NC_003552.1; NC_025263.1;
NC_016112.1; NC_011098.1; NC_007643.1;
NC_007643.1; NC_007643.1; NC_006347.1;
NC_005140.1; NC_004342.2; NC_002945.3;
NW_007382731.1; NW_007381138.1; NC_024320.1;
NW_005756335.1; NW_003384463.1; NC_019977.1;
NC_011296.1; NC_007929.1; NC_000913.3;
NC_003413.1; NC_002754.1; NC_010175.1;
NC_010175.1; NC_010175.1; NC_011661.1;
NC_014537.1; NC_012470.1; NC_004829.2;
NC_015516.1; NC_014374.1; NC_009033.1;
NC_007681.1; NC_002689.2; NC_006085.1;
NC_021592.1; NC_021592.1; NC_021169.1;
NC_020517.1; NC_018015.1; NC_018015.1;
NC_018015.1; NC_017946.1; NC_017946.1;
NC_017576.1; NC_017576.1; NC_015865.1;
NC_015865.1; NC_015847.1; NC_015680.1;
NC_015680.1; NC_015474.1; NC_015435.1;
NC_014106.1; NC_013790.1; NC_012883.1;
NC_012804.1; NC_016051.1; NC_011529.1;
NC_010482.1; NC_009515.1; NC_009440.1;
NC_008942.1; NC_008054.1; NC_007181.1;
NC_006624.1; NC_006448.1; NC_006448.1;
NC_002935.2; NC_002935.2; NC_002950.2;
NC_002663.1; NC_019943.1; NC_019943.1;
NC_017459.1; NC_017459.1; NC_016023.1;
NC_015518.1; NC_015460.1; NC_015460.1;
NC_015416.1; NC_014933.1; NC_013202.1;
NC_013158.1; NC_009464.1; NC_008508.1;
NC_003901.1; NC_009434.1; NC_005085.1;
NC_020247.1; NC_020246.1; NC_018224.1;
NC_015943.1; NC_009380.1; NC_006834.1;
NC_003552.1; NC_017276.1; NC_017275.1;
NC_010296.1; NC_009615.1; NC_012632.1;
NC_012632.1; NC_012623.1; NC_012588.1;
NC_012588.1; NC_007181.1; NC_002939.5;
NC_020247.1; NC_020246.1; NC_011296.1;
NC_011296.1; NC_011296.1; NC_018609.1;
NC_015964.1; NC_021355.1; NC_020800.1;
NC_019942.1; NC_019792.1; NC_015958.1;
NC_015760.1; NC_015678.1; NC_015636.1;
NC_015562.1; NC_014222.1; NC_014222.1;
NC_013887.1; NC_013769.1; NC_013156.1;
NC_009953.1; NC_009635.1; NC_009634.1;
NC_009135.1; NC_008618.1; NC_008599.1;
NC_007955.1; NC_007796.1; NC_007355.1;
NC_002754.1; NC_010572.1; NC_015151.1;
NC_000962.3; NC_021921.1; NC_002163.1;
NC_017034.1; NC_009089.1; NC_008698.1;
NC_020419.1; NC_020419.1; NC_020419.1;
NC_014160.1; NC_011766.1; NC_007681.1;
NC_000916.1; NC_017527.1; NC_013790.1;
NC_013790.1; NC_000917.1; NC_000917.1;
NC_004557.1; NC_004557.1; NC_022246.1;
NC_017384.1; NC_013722.1; NC_007643.1;
NC_007643.1; NC_007643.1; NC_007643.1;
NC_007643.1; NC_002967.9; NC_004342.2;
NC_016112.1; NC_016112.1; NC_005140.1;
NC_005140.1; NC_012726.1; NC_023069.1;
NC_023044.1; NC_022777.1; NC_022777.1;
NC_011296.1; NC_021355.1; NC_009634.1;
NC_007796.1; NC_007355.1; NC_021082.1;
NC_013926.1; NC_020913.1; NC_014961.1;
NC_014658.1; NC_013198.1; NC_005877.1;
NC_009785.1; NC_022084.1; NC_018092.1;
NC_014804.1; NC_000961.1; NC_021058.1;
NC_018081.1; NC_013849.1; NC_011567.1;
NC_015574.1; NC_014500.1; NC_012622.1;
NC_012589.1; NC_012589.1; NC_019977.1;
NC_019042.1; NC_017274.1; NC_017274.1;
NC_015954.1; NC_015676.1; NC_015320.1;
NC_014122.1; NC_014122.1; NC_013407.1;
NC_011835.1; NC_021353.1; NC_018001.1;
NC_008818.1; NC_000961.1; NC_015931.1;
NC_019042.1; NC_013961.1; NC_011138.3;
NC_009778.1; NC_014228.1; NC_013892.1;
NC_011832.1; NC_009439.1; NC_007955.1;
NC_007796.1; NC_013520.1; NC_016070.1;
NC_007426.1; NC_003106.2; NC_003106.2;
NC_018227.2; NC_000913.3; NC_005085.1;
NC_009613.3; NC_014334.1; NW_006726754.1;
NC_002663.1; NC_003143.1; NC_003076.8;
NC_015666.1; NC_014644.1; NC_004116.1;
NC_003454.1; NC_011567.1; NC_024905.1;
NC_003295.1; NC_008526.1; NC_012871.1;
NC_012871.1; NC_010682.1; NC_002737.2;
NC_002737.2; NC_017954.1; NC_009515.1;
NC_007297.1; NC_007297.1; NC_004350.2;
NC_004350.2; NC_000853.1; NC_009009.1;
NC_007644.1; NC_007644.1; NC_002967.9;
NC_002932.3; NC_002932.3; NC_007643.1;
NC_007606.1; NC_006347.1; NC_002945.3;
NW_006804726.1; NW_006383769.1; NC_013769.1;
NC_014644.1; NC_000913.3; NC_019943.1;
NC_019943.1; NC_011661.1; NC_010175.1;
NC_002950.2; NC_004547.2; NC_013887.1;
NC_013156.1; NC_007426.1; NC_002939.5;
NC_021169.1; NC_020517.1; NC_018015.1;
NC_017946.1; NC_015865.1; NC_015680.1;
NC_009515.1; NC_004557.1; NC_005085.1;
NC_006834.1; NC_011296.1; NC_010175.1;
NC_020800.1; NC_015958.1; NC_009635.1;
NC_008618.1; NC_007355.1; NC_009089.1;
NC_020419.1; NC_021592.1; NC_021592.1;
NC_015847.1; NC_013790.1; NC_016051.1;
NC_007644.1; NC_007644.1; NC_017459.1;
NC_015416.1; NC_013722.1; NC_007643.1;
NC_007643.1; NC_007643.1; NC_009434.1;
NC_005085.1; NC_003552.1; NC_014318.1;
NC_021355.1; NC_014222.1; NC_014222.1;
NC_011832.1; NC_009634.1; NC_009135.1;
NC_021082.1; NC_000961.1; NC_015574.1;
NC_014228.1; NC_014122.1; NC_009439.1;
NC_017459.1; NC_015460.1; NC_011138.3;
NC_009380.1; NC_017275.1; NC_013892.1;
NC_021353.1; NC_015676.1; NC_011296.1;
NC_007955.1; NC_009953.1; NC_009953.1;
NC_021921.1; NC_014160.1; NC_010482.1;
NC_009776.1; NC_009033.1; NC_016070.1;
NC_016070.1; NC_015435.1; NC_009440.1;
NC_017384.1; NC_013722.1; NC_016112.1;
NC_012726.1; NC_022777.1; NC_008698.1;
NC_008599.1; NC_007955.1; NC_007355.1;
NC_014147.1; NC_021058.1; NC_021058.1;
NC_016901.1; NC_014500.1; NC_014500.1;
NC_014961.1; NC_018001.1; NC_015931.1;
NC_015151.1; NC_013849.1; NC_013715.1;
NC_011766.1; NC_018001.1; NC_014644.1;
NC_017034.1; NC_009033.1; NC_002754.1;
NC_009089.1; NC_002939.5; NC_014106.1;
NC_010610.1; NC_008054.1; NC_003413.1;
NC_009464.1; NC_008526.1; NC_015474.1;
NC_012804.1; NC_015518.1; NC_017276.1;
NC_017275.1; NC_012632.1; NC_012623.1;
NC_012588.1; NC_015636.1; NC_015562.1;
NC_013769.1; NC_002754.1; NC_017634.1;
NC_014160.1; NC_011766.1; NC_016070.1;
NC_015435.1; NC_009440.1; NC_009440.1;
NC_012726.1; NC_012632.1; NC_012588.1;
NC_013887.1; NC_013156.1; NC_011296.1;
NC_002754.1; NC_011835.1; NC_018092.1;
NC_021058.1; NC_012622.1; NC_012589.1;
NC_015954.1; NC_013407.1; NC_018001.1;
NC_013849.1; NC_017274.1; NC_000913.3;
NC_003413.1; NC_018092.1; NC_000961.1;
NC_000918.1; NC_007796.1; NC_000868.1;
NC_022084.1; NC_018015.1; NC_015865.1;
NC_015680.1; NC_015474.1; NC_014804.1;
NC_012470.1; NC_006624.1; NC_002663.1;
NC_016023.1; NC_013202.1; NC_013158.1;
NC_008508.1; NC_000917.1; NC_015943.1;
NC_019792.1; NC_019042.1; NC_015760.1;
NC_015678.1; NC_014122.1; NC_004119.1;
NC_007681.1; NC_007681.1; NC_007297.1;
NC_002935.2; NC_002932.3; NC_003454.1;
NC_014933.1; NC_011567.1; NC_004342.2;
NC_016112.1; NC_003197.1; NC_022777.1;
NC_015320.1; NC_002695.1; NC_003143.1;
NC_002737.2; NC_012883.1; NC_010610.1;
NC_000916.1; NC_004350.2; NC_000853.1;
NC_000917.1; NC_006347.1; NC_018658.1;
NC_015870.2; NC_011751.1; NC_013961.1;
NC_009778.1; NC_020990.1; NC_016112.1;
NC_000868.1; NC_003413.1; NC_022084.1;
NC_018092.1; NC_017946.1; NC_015680.1;
NC_015680.1; NC_015474.1; NC_014106.1;
NC_012804.1; NC_009053.1; NC_008054.1;
NC_006624.1; NC_000961.1; NC_021058.1;
NC_015518.1; NC_018224.1; NC_017276.1;
NC_017276.1; NC_017275.1; NC_017275.1;
NC_010296.1; NC_010296.1; NC_009615.1;
NC_012632.1; NC_012632.1; NC_012632.1;
NC_012623.1; NC_012623.1; NC_012622.1;
NC_012622.1; NC_012589.1; NC_012589.1;
NC_012588.1; NC_012588.1; NC_012588.1;
NC_020892.1; NC_019792.1; NC_017970.1;
NC_017274.1; NC_017274.1; NC_016159.1;
NC_013887.1; NC_013769.1; NC_013156.1;
NC_002754.1; NC_002754.1; NC_002754.1;
NC_003687.1; NC_006814.3; NC_006814.3;
NC_014418.1; NC_010152.1; NC_017946.1;
NC_017954.1; NC_009776.1; NC_008818.1;
NC_008818.1; NC_000961.1; NC_000918.1;
NC_015931.1; NC_015931.1; NC_014537.1;
NC_007181.1; NC_006624.1; NC_003106.2;
NC_004342.2; NC_020247.1; NC_020246.1;
NC_018472.1; NC_012623.1; NC_012589.1;
NC_006045.2; NC_023069.1; NC_022777.1;
NC_019942.1; NC_017274.1; NC_013769.1;
NC_009953.1; NC_008698.1; NC_007493.2;
NC_002754.1; NC_002754.1; NC_005125.1;
NC_021347.1; NC_022093.1; NC_022093.1;
NC_015931.1; NC_007164.1; NC_015416.1;
NC_015151.1; NC_000917.1; NC_003106.2;
NC_002932.3; NC_014500.1; NC_004337.2;
NC_007087.3; NC_012726.1; NC_024314.1;
NW_003120284.1; NW_003120529.1; NW_003126883.1;
NW_003384275.1; NC_023069.1; NC_016567.1;
NC_009954.1; NC_000913.3; NC_000913.3;
NC_000913.3; NC_000913.3; NC_027204.1;
NC_002754.1; NC_010175.1; NC_016070.1;
NC_000868.1; NC_003413.1; NC_017527.1;
NC_002939.5; NC_018227.2; NC_007355.1;
NC_014205.1; NC_014160.1; NC_009033.1;
NC_007681.1; NC_020517.1; NC_018015.1;
NC_016070.1; NC_015865.1; NC_015847.1;
NC_015680.1; NC_015474.1; NC_015315.1;
NC_013790.1; NC_012883.1; NC_012470.1;
NC_016051.1; NC_011529.1; NC_010610.1;
NC_009515.1; NC_009440.1; NC_007181.1;
NC_006624.1; NC_019943.1; NC_019943.1;
NC_017459.1; NC_015518.1; NC_014933.1;
NC_007426.1; NC_003901.1; NC_003106.2;
NC_003106.2; NC_009434.1; NC_005085.1;
NC_020247.1; NC_020246.1; NC_006834.1;
NC_017276.1; NC_013158.1; NC_000917.1;
NC_020247.1; NC_020246.1; NC_003552.1;
NC_017275.1; NC_010296.1; NC_012632.1;
NC_012632.1; NC_012588.1; NC_012588.1;
NC_011296.1; NC_011296.1; NC_021355.1;
NC_019792.1; NC_015958.1; NC_015636.1;
NC_015562.1; NC_013887.1; NC_013156.1;
NC_007955.1; NC_007355.1; NC_002754.1;
NC_002754.1; NC_009778.1; NC_000962.3;
NC_009089.1; NC_021592.1; NC_017946.1;
NC_015680.1; NC_018015.1; NC_014537.1;
NC_014537.1; NC_012883.1; NC_012804.1;
NC_018224.1; NC_017459.1; NC_016023.1;
NC_015943.1; NC_023069.1; NC_023044.1;
NC_019942.1; NC_014222.1; NC_008599.1;
NC_002754.1; NC_022084.1; NC_022084.1;
NC_022084.1; NC_018092.1; NC_014804.1;
NC_014804.1; NC_021058.1; NC_017274.1;
NC_015320.1; NC_014122.1; NC_013407.1;
NC_014658.1; NC_000961.1; NC_000961.1;
NC_018092.1; NC_015151.1; NC_013849.1;
NC_011567.1; NC_013926.1; NC_002754.1;
NC_013520.1; NC_013520.1; NC_007181.1;
NC_007426.1; NC_003106.2; NC_020247.1;
NC_020246.1; NC_017276.1; NC_017275.1;
NC_012632.1; NC_012623.1; NC_012588.1;
NC_011296.1; NC_011296.1; NC_013769.1;
NC_013769.1; NC_012726.1; NC_021058.1;
NC_012622.1; NC_012589.1; NC_015151.1;
NC_015954.1; NC_004547.2; NC_000913.3;
NC_010175.1; NC_016070.1; NC_002950.2;
NC_009380.1; NC_009089.1; NC_009495.1;
NC_022777.1; NC_007796.1; NC_014106.1;
NC_008054.1; NC_002663.1; NC_013961.1;
NC_011138.3; NC_014228.1; NC_020990.1;
NC_013892.1; NC_015760.1; NC_009953.1;
NC_009953.1; NC_009439.1; NC_010572.1;
NC_002971.3; NC_021353.1; NC_014644.1;
NC_010610.1; NC_002935.2; NC_013722.1;
NC_009464.1; NC_007643.1; NC_002939.5;
NC_010002.1; NC_003198.1; NC_014318.1;
NC_008526.1; NC_011832.1; NC_008701.1;
NC_007955.1; NC_007796.1; NC_014147.1;
NC_009053.1; NC_014500.1; NC_016901.1;
NC_015676.1; NC_020418.1; NW_003613864.1;
NC_000011.10; NW_006800487.1; NC_006603.3;
NW_003614246.1; NC_012602.1; NC_013790.1;
NC_009515.1; NC_015574.1; NC_007871.1;
NC_006478.3; NC_004347.2; NC_009006.2;
NT_078266.2; NC_003454.1; NW_006212882.1;
NC_011913.1; NC_009917.1; NW_007675828.1;
NW_007370782.1; NW_007248774.1; NC_023642.1;
NW_006775074.1; NW_006730123.1; NW_006718075.1;
NW_006711808.1; NW_006400147.1;
NW_006408681.1; NW_006384369.1;
NW_005882764.1; NW_006200097.1; NC_022285.1;
NW_004209914.1; NC_018435.1; NC_018732.2;
NC_018165.1; NC_027879.1; NC_019830.1;
NC_013906.1; NC_009150.2; NC_000964.3;
NC_002696.2; NC_017034.1; NC_007581.1;
NC_018001.1; NC_017954.1; NC_014961.1;
NC_014961.1; NC_011766.1; NC_009776.1;
NC_007681.1; NC_007681.1; NC_005877.1;
NC_005877.1; NC_005877.1; NC_002689.2;
NC_000918.1; NC_000918.1; NC_021592.1;
NC_015931.1; NC_015931.1; NC_010482.1;
NC_009033.1; NC_000853.1; NC_015518.1;
NC_015416.1; NC_013849.1; NC_009440.1;
NC_009440.1; NC_009009.1; NC_007644.1;
NC_000917.1; NC_003106.2; NC_011916.1;
NC_007643.1; NC_006347.1; NC_004342.2;
NC_002945.3; NC_012589.1; NC_012623.1;
NC_011672.1; NC_016131.1; NW_004454187.1;
NC_019862.1; NC_010451.3; NC_015768.1;
NC_020173.2; NC_017972.1; NC_015320.1;
NC_011832.1; NC_010175.1; NC_010175.1;
NC_008599.1; NC_006461.1; NC_015637.1;
NC_009784.1; NC_016114.1; NC_001493.2;
NC_008508.1; NC_003197.1; NC_017844.1;
NW_006399893.1; NC_002695.1; NC_017634.1;
NC_003143.1; NC_017941.2; NC_004605.1;
NC_004605.1; NC_019411.1; NC_007164.1;
NC_002932.3; NC_005085.1; NC_027207.1;
NC_016452.1; NC_016112.1; NC_009784.1.

In some embodiments, a vector encodes a CRISPR enzyme comprising one or more nuclear localization sequences (NLSs), such as about (or more than about) 1, 2, 3, 4, 5, 6, 7, 8, 9, 10, or more NLSs. In some embodiments, the CRISPR enzyme comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g. one or more NLS at the amino-terminus and one or more NLS at the carboxy terminus). When more than one NLS is present, each may be selected independently of the others, such that a single NLS may be present in more than one copy and/or in combination with one or more other NLSs present in one or more copies. In a preferred embodiment of the disclosure, the CRISPR enzyme comprises at most 6 NLSs. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus.

Some embodiments of the disclosure relate to use of transposases as a gene-editing enzyme or nucleic acid sequences encoding the same. When compared with viral transduction of immune cells, such as B lymphocytes, delivery of transgenes via DNA transposases, such as piggyBac and Sleeping Beauty, offers significant advantages in ease of use, ability to delivery much larger cargo, speed to clinic and cost of production. The piggyBac DNA transposase, in particular, offers additional advantages in giving long-term, high-level and stable expression of transgenes, and in being significantly less mutagenic than a retrovirus, being non-oncogenic and being fully reversible. For example, the poor efficiency demonstrated by previous methods of using DNA transposase to deliver transgenes to T cells has resulted in the need for prolonged expansion ex vivo. Previous unsuccessful attempts by others to solve this problem have all focused on increasing the amount of DNA [symbol] sposase delivered to the immune cell, which has been a strategy that worked well for non-immune cells. This disclosure relates to methods of decreasing the amount of DNA delivered to the immune cell. Using the methods of the disclosure, the data provided herein demonstrate not only that decreasing the amount of [symbol] transposase introduced into the cell increased viability but also that this method increased the percentage of cells that harbored a transposition event, resulting in a viable commercial process and a viable transduction of B cells and T cells.

In certain embodiments, the immune cell is isolated or derived from a non-human primate. In certain embodiments of the methods of the disclosure, the transposase enzyme is a SuperPiggyBac (sPBo) transposase enzyme. The Super piggyBac (PB) transposase enzyme may comprise or consist of an amino acid sequence comprising at least about 75% sequence identity to: MGSSLDDEHILSALLQSDDELVGEDSDSEVSDHVSEDDVQSDTEEAFIDEVHEVQPTS SGSEILDEQNVIEQPGSSLASNRILTLPQRTIRGKNKHCWSTSKSTRRSRVSALNIVRSQRG PTPJVICRNIYDPLLCFKLFFTDEIISEIVKWTNAEISLKRRESMTSATFRDTNEDEIYAFFGI LVMTAVRKDNHMSTDDLFDRSLSMVYVSVMSRDRFDFLIRCLRMDDKSIRPTLRENDVF TPVRKIWDLFIHQCIQNYTPGAHLTIDEQLLGFRGRCPFRVYIPNKPSKYGIKILMMCDSG TKYMINGMPYLGRGTQTNGVPLGEYYVKELSKPVHGSCRNITCDNWFTSIPLAK LLQEPYKLTIVGTVRSNKREIPEVLKSRSRPVGTSMFCFDGPLTLVSYKPKPAIGVIVYLLS SCDEDASINESTGKPQMVMYYNQTKGGVDTLDQMCSVMTCSRKTORWPMALLYGMIN IACINSFIIYSHNVSSKGEKVQSPJ KFMRNLYMSLTSSFMRKRLE APTLKRYLRDNISNILPKEVPGTS DD STEEP VMKKRTYCTYCP S KIRRKAN AS CKKCKKVICREHNIDMCQSCF

In certain embodiments of the methods and compositions of the disclosure, the gene-editing enzyme is a Sleeping Beauty transposase enzyme (see, for example, U.S. Pat. No. 9,228,180, the contents of which are incorporated herein in their entirety). In certain embodiments, the Sleeping Beauty transposase is a hyperactive Sleeping Beauty SB100× transposase. In certain embodiments, the Sleeping Beauty transposase enzyme comprises an amino acid sequence comprising at least about 75% sequence identity to:

MGKS KEI S QDLRKKIVDLHKS GS S LGAIS KRLKVPRS
SVQTIVRKYKHHGTTQPSYRSGRRRYLSPRDERTLVRKVQINPRTTAKDL
VKMLEETGTKVSISTVKRVLYRHNLKGRSARKKPLLQNP^KKARLRFATA
HGDKDRTFWRNVLWSDETKIELFGHNDHRYVWRKKGEACKPKNTIPTVKH
GGGSIMLWGCFAAGGTGALHKIDGIMRKENYVDILKQHLKTSVRKLKLGR
KWVFQMDNDPKHTSKVVAKWLKDNKVKVLEWPSQSPDLNPIENLWAELKK
RVRARRPTNLTQLHQLCQEEWAKIHPTYCGKLVEGYPKRLTQVKQFKGNA
TKY.

Some embodiments comprise a nucleic acid sequence encoding the gene-editing enzyme or a functional variant thereof.

In certain embodiments, including those wherein the SleepingBeauty transposase is a hyperactive Sleeping Beauty SBIOOX transposase, the SleepingBeauty transposase enzyme comprises an amino acid sequence at least about 75% sequence identity to:

(SEQ ID NO: 3)
MGKSKEISQDLRKRIVDLHKSGSSLGAISKRLAVPRSSVQTIVRKYKHH
GTTQPSYRSGRRRYLSPRDERTLVRKVQINPRTTAKDLVKMLEETGTKV
SISTVKRVLYRHNLKGHSARKKPLLQNRHKKARLRFATAHGDKDRTFWR
NVLWSDETKIELFGHNDHRYVWRKKGEACKPKTIPTVKHGGGSIMLWGC
FAAGGTGALHKIDGIMDAVQYVDILKQHLKTSVRKLKLGRKWVFQHDND
PKHTSKVVAKWLKDNKVKVLEWPSQSPDLNPIENLWAELKKRVRARRPT
NLTQLHQLCQEEWAKIHPNYCGKLVEGYPKRLTQVKQFKGNATKY

In certain embodiments, the recombinant and non-naturally occurring DNA sequence comprising a DNA sequence encoding a transposon may be circular. As a nonlimiting example, the DNA sequence encoding a transposon may be a plasmid vector. As a nonlimiting example, the DNA sequence encoding a transposon may be a minicircle DNA vector. In certain embodiments of the methods of the disclosure, the recombinant and non-naturally occurring DNA sequence encoding a transposon may be linear. The linear recombinant and non-naturally occurring DNA sequence encoding a transposon may be produced in vitro. Linear recombinant and non-naturally occurring DNA sequences of the disclosure may be a product of a restriction digest of a circular DNA. In certain embodiments, the circular DNA is a plasmid vector or a minicircle DNA vector. Linear recombinant and non-naturally occurring DNA sequences of the disclosure may be a product of a polymerase chain reaction (PCR). In some embodiments, the nucleic acid sequence encoding the transposase appear on a second nucleic acid molecule exposed to the target cell under conditions conducive to transduction or on the same nucleic acid molecule comprising the barcode domain.

The nucleic acid molecules of some embodiments of the disclosure also comprises a first and a second gene editing enzyme recognition sequence, or interchangeably, a gene editing enzyme cleavage site. Transposase recognition sites are known in the art. A non-limiting examples of SleepingBeauty transposon sequences are as follows:

The 5′ outer repeat:
5′-GTTGAAGTCGGAAGTTTACATACACTTAAG-3′
The 5′ inner repeat:
5′-CAGTGQQTCAGAAGTTTACATACACTAAGG-3′
The 3′ inner repeat
5′-CAGTGGGTCAGAAGTTAACATACACTCAATT-3′
The 3′ outer repeat
5′-AGTTGAAGTCGGAAGTTTACATACACCTTAG-3′.

A preferred consensus direct repeat for SleepingBeauty activity is:

5′-CA(GT)TG(AG)GTC(AG)GAAGTTTACATACACTTAAG-3′

In one embodiment the direct repeat sequence includes at least the following sequence: ACATACAC

In some embodiments the first inverted repeat sequence, or cleavage recognition site is as follows or comprises a nucleic acid sequence comprising at least about 75%, 80%, 85%, 90%, 91%, 92%, 93%, 94%, 95%, 96%, 97%, 98%, or 99% sequence identity to:

5′-AGTTGAAGTC GGAAGTTTAC ATACACTTAA GTTGGAGTCA
TTAAAACTCG TTTTTCAACT ACACCACAAA TTTCTTGTTA
ACAAACAATA GTTTTGGCAA GTCAGTTAGG ACATCTACTT
TGTGCATGAC ACAAGTCATT TTTCCAACAA TTGTTTACAG
ACAGATTATT TCACTTATAA TTCACTGTAT CACAATTCCA
GTGGGTCAGA AGTTTACATA CACTAA-3′

In some embodiments, the second inverted repeat sequence is as follows or comprises a nucleic acid sequence comprising at least about 75%, 80%, 85%, 90%, 91%, 92%, 93%, 94%, 95%, 96%, 97%, 98%, or 99% sequence identity to:

5′-TTGAGTGTAT GTTAACTTCT GACCCACTGG GAATGTGATG
AAAGAAATAA AGCTGAAAT GAATCATTCT CTCTACTATT
ATTCTGATAT TTCACATTCT TAAAATAAAG TGGTGATCCT
AACTGACCTT AAGACAGGGA ATCTTTACTC GGATTAAATG
TCAGGAATTG TGAAAAAGTG AGTTTAAATG TATTTGGCTA
AGGTGTATGT AAACTTCCGA CTTCAACTG-3′.

The direct repeats are the portion of the inverted repeat that bind to the SB protein to permit insertion and integration of the nucleic acid fragment into the cell. The site of DNA integration for the SB proteins occurs at TA base pairs.

The inverted repeats flank a nucleic acid sequence comprising a barcode which is inserted into the DNA in a cell. The nucleic acid sequence can include all or part of an open reading from of a gene (i.e., that part of a gene encoding protein), one or more regulatory sequences alone or together with all or part of an open reading frame. Preferred expression control sequences include, but are not limited to promoters, enhancers, border control elements, locus-control regions or silencers. In another set of embodiments, the nucleic acid sequence comprises a promoter operably linked to at least a portion of an open reading frame. In some embodiments, the nucleic acid sequence encoding a gene editing enzyme or functional variant thereof encodes a transposase, meganuclease, or Cas protein; and wherein the first gene editing enzyme cleavage sequence and a second gene editing enzyme cleavage sequence is a transposase, meganuclease, or Cas protein recognition and cleavage site, respectively. In some embodiments, the nucleic acid sequence encoding a gene editing enzyme or functional variant thereof encodes a transposase or functional variant thereof. In some embodiments, the gene editing enzyme cleavage sequence is a gene editing enzyme recognition sequence through which the enzyme binds to the DNA and excises the DNA.

Generally, in 5′ to 3′ orientation, the engineered AAV genome comprises:
(i) the nucleic acid sequence encoding a gene editing enzyme or functional variant thereof;
(ii) the first gene editing enzyme cleavage sequence;
(iii) the barcode sequence; and
(iv) the second gene editing enzyme cleavage sequence. In some embodiments, the nucleic acid sequence comprises a pair of AAV ITRs that are both at the end or proximate to the 5′ and 3′ end of the nucleic acid molecule.

The nucleic acid molecule comprises a first and second inverted tandem repeat (ITR) sequence positioned at the 5′ end and 3′ end of the nucleic acid molecule, respectively. In some embodiments, the first and second inverted tandem repeat sequences comprise AAV ITR sequences: tcgactctga cggttcacta aacgagctct gcttatatag caacctgagt gatggctccg cccacgcgtg cacgtcaccg ttaceggagc aacctgacac cggctccagc [SEQ ID NO:1]

In some embodiments, the regulatory sequence is positioned about 50 nucleotides upstream from the 5′ end of the nucleic acid sequence encoding the gene editing enzyme or functional variant thereof. In some embodiments, the regulatory sequence is a cytomegalovirus promoter.

In some embodiments, the transposase is SleepingBeauty, PiggyBac, or a functional variant thereof and the first and second gene editing enzyme cleavage sequences comprise SleepingBeauty cleavage sequences or PiggyBac cleavage sequences. In some embodiments, the transposase is a modified Sleeping Beauty, SB100×: PSGHSARKKPLLQNRHKKARLRFATAHGDKDRTFWRNVLWSDETKIELFGHNDHRYVW RKKGEACKPKNTIPTVKHGGGSIMLWGCFAAGGTGALHKIDGIMDAVQYVDILKOHLK TSVRKLKLGRKWVFQHDNDPKHTSKVVAKWLKDNKVKVLEWPSQSPDLNPIENLWAE LKKRVRARRPTNLTQLHQLCQEEWAKIHPNYCGKLVEGYPKRLTQVKQFKGNATKY [SEQ ID NO 2], and the first and second gene editing enzyme cleavage sequences comprise SleepingBeauty cleavage sequences:

[SEQ ID NO. 3]
GAGCTCGGTACCCTATACAGTTGAAGTCGGAAGTTTACATACACTTAA
GTTG

In some embodiments, the nucleic acid encoding a gene editing enzyme or functional variant thereof comprises a polyA tail sequence. In some embodiments, the poly A tail sequence is an SV40 polyA tail.

In a second aspect, the disclosure provides a liposome, cell or virus comprising the nucleic acid molecule according to the first aspect of the disclosure, wherein the nucleic acid molecule is positioned within the liposome, cell or virus.

In some embodiments, the virus is an AAV pseudovirus. In some embodiments, the AAV pseudovirus is replication deficient.

In some embodiments, the cell comprises a barcode DNA within its genome that is detectable by PCR, qPCR and/or NGS. In some embodiments, further comprising either (i) a second vector expressing AAV capsid proteins and a third vector expressing AAV helper proteins, or (ii) a second vector expressing AAV capsid proteins and AAV helper proteins. In some embodiments, the cell further comprises an antigen, or one or more epitope from the antigen, fused to a ligand specific for the ligand binding protein or peptide, wherein the cell comprising the antigen or one or more epitope from the antigen is differentially barcoded from cells comprising a different antigen or one or more different epitope of the antigen.

In some embodiments, the cell is a B cell. In some embodiments, the B cell is a human cell. In some embodiments, the cell is a T cell. In some embodiments, T cell is a human cell.

In some embodiments, the cell further comprises either (i) a second vector expressing AAV capsid proteins and a third vector expressing AAV helper proteins, or (ii) a second vector expressing AAV capsid proteins and AAV helper proteins.

### Methods

The disclosure provides non-immunogenic method to label a B cell with a DNA barcode, or aptamer, in order to screen, select and/or isolate epitopes of immunogens in the B cells. The disclosure relates to (a) exposing a cell to a viral particle comprising:
(i) a barcode sequence; and
(ii) a first and a second gene editing enzyme cleavage sequence; and
(iii) a pair of viral ITRs that are positioned at the end of the nucleic acid molecule or proximate to the 5′ and 3′ end of the nucleic acid molecule. The disclosure further relates to a method comprising: (a) exposing a target cell, such as an immune cell, to a viral particle comprising a nucleic acid molecule comprising:
(i) a barcode sequence; and
(ii) a first and a second gene editing enzyme cleavage sequence;
(iii) a pair of viral ITRs that are positioned at the end of the nucleic acid molecule or proximate to the 5′ and 3′ end of the nucleic acid molecule; and
(iv) a nucleic acid sequence encoding a gene editing enzyme or functional variant thereof; wherein the step of exposing is performed for a time period sufficient to allow entry of the virus into the target cell. In some embodiments, the method further comprises a step of (b) allowing a time period for a gene editing enzyme to excise the barcode sequence from the nucleic acid molecule and edit genomic DNA of the cell such that the barcode sequence is stably integrated into the genomic DNA of the cell. In some embodiments, the cell is a B cell. In some embodiments, the viral particle is replication deficient and infection-deficient. In some embodiments, the viral particle is an AAV pseudotype comprising an AAV vector comprising:
(i) a barcode sequence; and
(ii) a first and a second gene editing enzyme cleavage sequence;
(iii) a pair of viral ITRs that are positioned at the end of the nucleic acid molecule or proximate to the 5′ and 3′ end of the nucleic acid molecule; and the viral particle surface comprises a modified VP1, VP2 and VP3 polypeptide that confers inability of the viral particle to infect a cell through heparan sulfate on a target cell and an antigen or functional variant thereof. Some embodiments further comprise the step of allowing the antigen to elicit an antigen-specific immune response; measuring the immune response and identifying the cell by its barcode sequence.

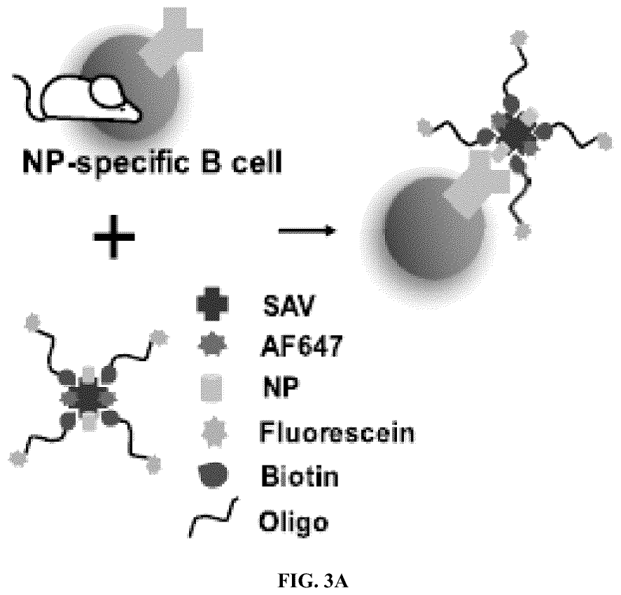

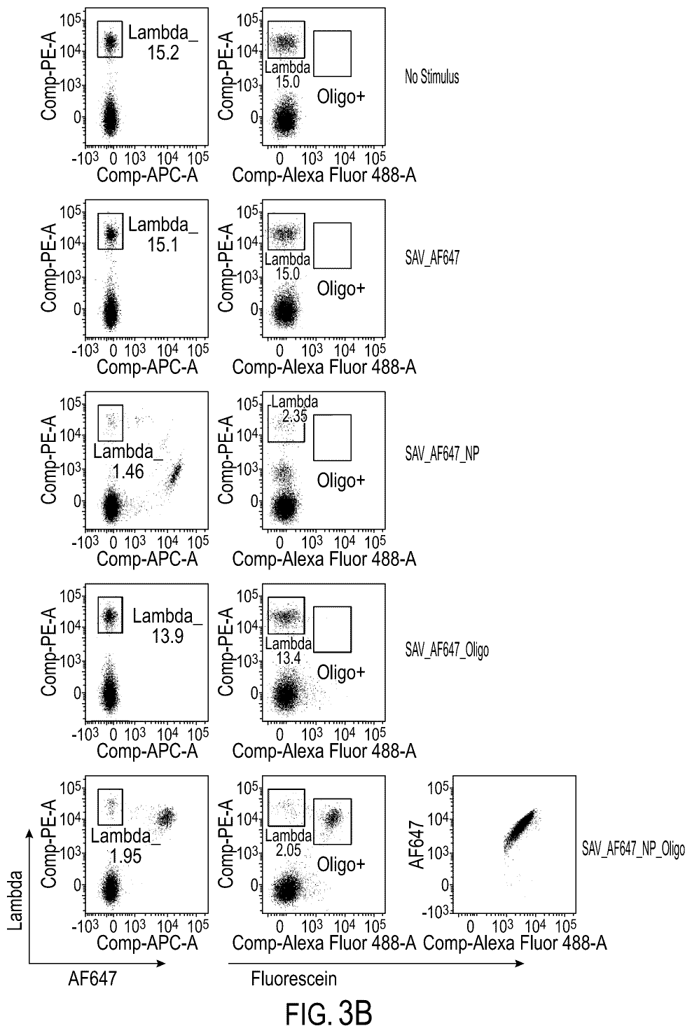

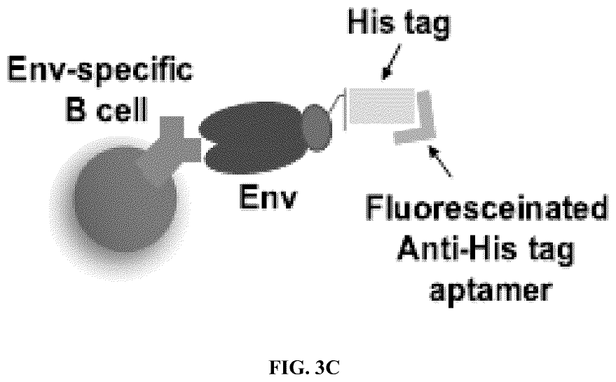

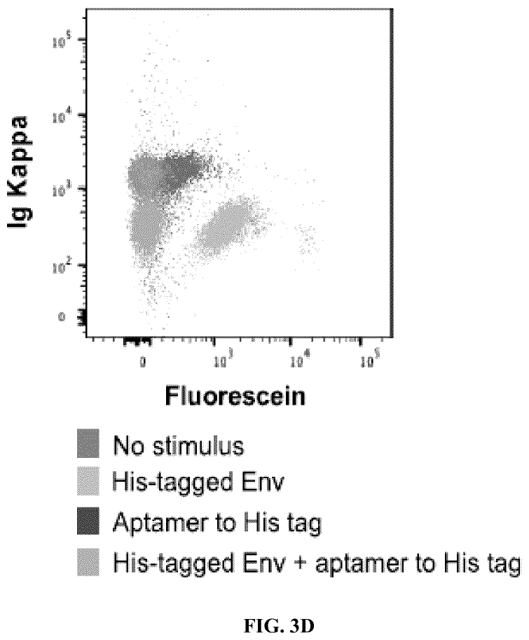

The barcodes can be integrated into the genome of the cells using methods known in the art, e.g., viral delivery vectors, e.g., retroviral or lentiviral vectors, as known in the art and described herein to achieve stable integration. Other methods can also be used, e.g., homologous or targeted integration, or integration using a recombinase such as the Cre-Lox, Flp-FRT, and zinc-finger recombinases (ZFRs); piggyBac and Sleeping Beauty transposon systems; and others. See, e.g., Gersbach et al., Nucleic Acids Research, 2011, 1-11 (doi: 10.1093/nar/gkr421); Wilson et al., Mol. Ther. 2007; 15:139-145; VandenDriessche et al., Blood 2009; 114:1461-1468; Bushman et al., Nat. Rev. Microbiol. 2005; 3:848-858; Yant et al., Nucleic Acids Res. 2007; 35: e50; Sauer et al., Proc. Natl Acad. Sci. USA 1988; 85:5166-5170; Logie et al., Proc. Natl Acad. Sci. USA 1995; 92:5940-5944; Thyagarajan et al., Mol. Cell. Biol. 2001; 21:3926-3934; Wu et al., Proc. Natl Acad. Sci. USA 2006; 103:15008-15013; and others. Antigen-specific barcodes can be integrated within immune cells are produced using the method called Systematic Evolution of Ligands by Exponential Enrichment (SELEX) (FIG. 3B). For SELEX, the antigen of interest is incubated with a pool of 1014-1016 random single-stranded oligonucleotides of typically 40-100 nucleotides containing a random region in the middle and fixed primer-annealing sequences on both ends. Non-binding oligonucleotides are discarded, and the antigen-binding oligonucleotides are eluted and amplified by PCR. This cycle is repeated multiple times, and after several rounds of selection, the resulting DNA sequences, with high affinity and specificity for the antigen, are enriched in the pool and sequenced. Either naked antigens, or antigens bound to Fabs protecting the conserved epitopes of interest, will be used as targets for SELEX. Selected barcodes will be characterized for their binding epitopes, and the antigen-barcode correspondences will be evaluated as immunogens in mice.

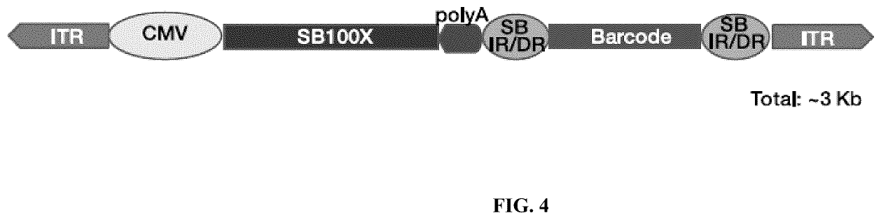

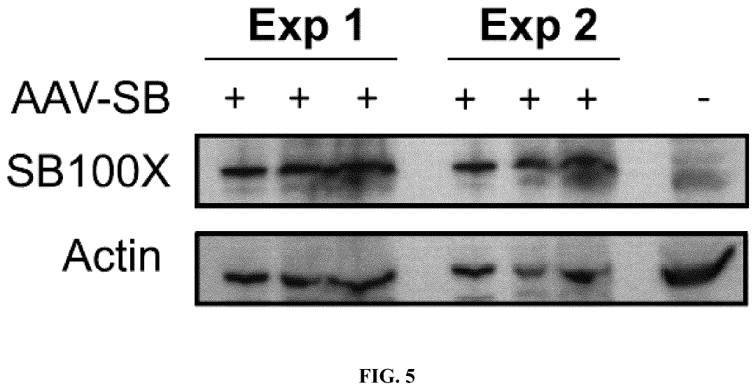

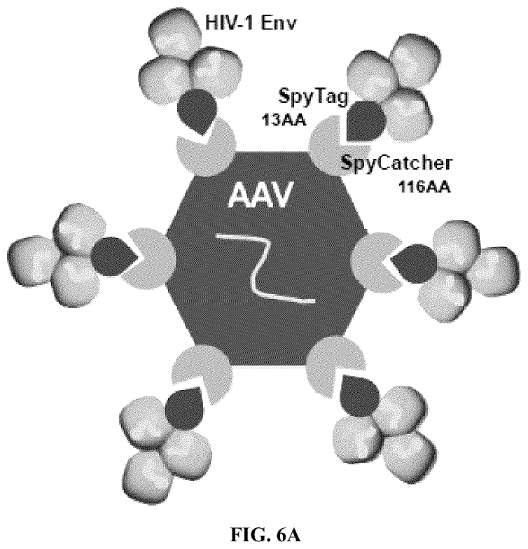

An additional bonus of the barcode technology is that it can be used for high-throughput mapping of BCRs in a similar manner to the previously reported LIBRA-seq method²⁸. LIBRA-seq uses random ssDNA tails to barcode antigens, which are subsequently identified by Next Generation Sequencing (NGS) and used to identify antigen-specific B cells. Using our technology, we can take advantage of the barcode-specific binding to antigen. Aptamers will be internalized by the cell with their specific antigens upon antigen-BCR interaction, and the aptamer and BCR sequences will be then determined by NGS. Barcode technology will allow to identify antigen- and epitope-specific B cells in large pools of cells, such as blood samples collected from virus infected individuals. This will be a great asset to studies aiming to isolate bNAbs. Incubating B cells with different antigen-barcode complexes, followed by barcode detection and sequencing in single B cells, will allow methods of classifying B cells according to their antigen or epitope specificity (FIG. 4). The barcode technology can then be used as a screening method to identify antibodies of therapeutic interest. In summary, barcode technology in combination with single-B-cell antibody sequencing, will be very valuable for the characterization of antibody responses to vaccination and infection. Methods of the disclosure relate to methods of identifying antigen-specific B cells. ELISAs, Biolayer Interferometry (BLI) and other binding assays are performed to confirm the binding of the antigens to their corresponding targets. Antibody responses elicited upon immunization with antigen complexes are analyzed, and a combination of direct and competition ELISAs using the serum of the immunized animals are used to map the specific epitope activation of the elicited antibody responses. Single B cells are isolated from the immunized animals, cloned and used to produce their specific antibodies. Vaccine elicited antibodies are characterized in ELISAs, neutralization assays and by structural studies. B cells that are tagged with barcodes can be detected upon internalization. Fluorescently labelled ssDNA oligos and antigen complexes are used in combination with flow cytometry, PCR and NGS sequencing approaches to detect barcodes inside single B cells genomic DNA.

The disclosure relates to a method of reducing or abrogating an immune response by viral display of antigen with aptamer sequence that may mask immunodominant epitopes that are not therapeutic. In some embodiments, the method comprises exposing a target cell to a viral particle disclosed herein to an aptamer sequence, such that the aptamer sequence is positioned on the antigen domain and then subsequently exposing the viral particle to an immune cell for a time period sufficient to induce an antigen-specific immune response. The method may alternatively comprise the step of: (a) exposing a target cell, such as an immune cell, to a viral particle comprising an antigen or fragment thereof and a nucleic acid molecule comprising:
(i) a barcode sequence; and
(ii) a first and a second gene editing enzyme cleavage sequence;
(iii) a pair of viral ITRs that are positioned at the end of the nucleic acid molecule or proximate to the 5′ and 3′ end of the nucleic acid molecule; and
(iv) a nucleic acid sequence encoding a gene editing enzyme or functional variant thereof; wherein the step of exposing is performed for a time period sufficient to allow entry of the virus into the target cell; and (b) exposing the target cell to an aptamer or plurality of aptamers.

An object of the disclosure is to identify antigen-specific B cells. The detection of integrated barcodes identifies antigen-specific B cells with no need of cloning their antibody genes or using bait-based FACS. Methods of the disclosure are free of steps flow cytometry and free of cloning expressible antibody or antibody fragment nucleic acid sequences. The use of differentially barcoded pseudotyped AAV-SBs as stimuli in vitro or as immunogens in vivo, allows classification of B cells according to their antigen recognition profiles. This is of particular relevance to identify antigen-specific plasma B cells differentiated upon stimulation with the pseudotyped AAV-SB. Plasma B cells downregulate the expression of BCR on their surface, thus, bait-based FACS cannot be used for identification and isolation.

Another object of the disclosure is epitope mapping. AAV-SBs pseudotyped with different immunogen subunits or mutants, for example the gp120 and gp41 subunits of Env, or the globular head and the stem of influenza hemagglutinin, is used to determine the epitope specificity of B cells with no need of antibody cloning and production.

Another object of the disclosure is designing sequential immunization protocols. Methods of the disclosure include methods to classify B cells according to their history of antigen encounters upon sequential immunization with one or more antigens. For instance, by using differentially barcoded AAV-SBs as sequential antigens, it is possible to determine what antigen or epitope within the antigen initiated the maturation of a bNAb lineage, what immunogen led to a dead-end for a lineage, or what series of immunogens was more efficient at boosting the response elicited by a priming immunogen.

Another object of the disclosure is studying B cell fate upon immunization. The disclosure allows investigation of the role of the affinity, dose, and formulation of the immunogen on the differentiation of B cells towards the memory or plasma B cell compartments. As an example, mixtures of differentially barcoded AAV-SBs, pseudotyped with antigens of different affinities for the antibody of an Ig KI mouse, will be used to determine what antigen-induced preferential differentiation towards memory or plasma cells. This is greatly relevant to design vaccines that efficiently induce a long-lasting memory response.

Another object of the disclosure is studying memory B cell recall upon sequential cell activation with antigen or antigen fragments. The role of memory B cells in secondary immune responses is a focus of intense research. There are no available tools to identify memory B cells that responded to immunization multiple times. The present disclosure will allow identification of memory B cells and determine their history of antigen encounters permanently imprinted by the integration of different antigen-specific barcodes. This will be very useful to identify series of immunogens that efficiently induced the reactivation of memory B cells, thus, inducing further antibody maturation and prolonged protection.

Another object of the disclosure is studying immunodominance. Immunization with mixtures of differentially barcoded AAV-SBs, pseudotyped with epitope mutant proteins will allow identification of immunodominant epitopes. The results of these experiments will significantly contribute to immunogen design efforts aiming to focus antibody responses to conserved epitopes of interest.

Another object of the disclosure is studying B cell clonality. Barcode insertion sites are used to identify and characterize B cell clones.

These and other objects will be recognized by the following non-limiting aspects of the disclosure.

In some aspects, the disclosure provides a vaccine comprising a virus particle produced by the cell according to the second aspect according to the disclosure. In some embodiments, the vaccine further comprises an inhibitor of the transposase. In some embodiments, the vaccine further comprises one or a plurality of aptamers that mask one or a plurality of variable immunodominant epitopes on the antigen. In some embodiments, the ligand binding protein is SpyCatcher or a functional variant thereof and the ligand is Spy Tag. In some embodiments, the inhibitor of the transposase is an inhibitor of expression of the transposase. In some embodiments, the inhibitor of expression of the transposase is a small interfering RNA (siRNA) specific for an mRNA encoding the transposase.

In some embodiments, the kit further comprises (iii) a helper plasmid. In some embodiments the kit further comprising a transfection buffer.

In some embodiments, the disclosure provides a method for manufacturing a vaccine comprising transfecting cells with a transfection mixture comprising the nucleic acid according to the first aspect of the disclosure and a plurality of expression vectors expressing fragments of an antigen fused to a ligand that specifically bind to the ligand-binding protein or peptide, wherein the transfected cells are differentially barcoded according to the fragment of the antigen they bind to, wherein the gene editing enzyme is a transposase. In some embodiments, the transposase is Sleeping Beauty or SB100× or a functional variant thereof. In some embodiments, the ligand binding protein is SpyCatcher, encoded by SEQ ID NO:4:

[SEQ ID NO 5]
atgtcgtactaccatcaccatcaccatcacgattacgacatcccaacga
ccgaaaacctgtattttcagggcgccatggttgataccttatcaggttt
atcaagtgagcaaggtcagtccggtgatatgacaattgaagaagatagt
gctacccatattaaattctcaaaacgtgatgaggacggcaaagagttag
ctggtgcaactatggagttgcgtgattcatctggtaaaactattagtac
atggatttcagatggacaagtgaaagatttctacctgtatccaggaaaa
tatacatttgtcgaaaccgcagcaccagacggttatgaggtagcaactg
ctattacctttacagttaatgagcaaggtcaggttactgtaaatggcaa
agcaactaaaggtgacgctcatatttaa, or a functional
variant thereof and the ligand is SpyTag:
AHIVMVDAYKPTK.

In some embodiments, the disclosure provides a method for labeling an immune cell with a barcode comprising:
(a) exposing a population of immune cells to a transfection mixture comprising the vaccine according to the third aspect of the disclosure and a non-toxic carrier or diluent for a time period sufficient for the virus particle to enter the cells; and
(b) exposing the barcode DNA to the gene editing enzyme for a time period sufficient for the gene editing enzyme to excise the nucleic acid sequence encoding the barcode; and integrate the nucleic acid sequence encoding the barcode into genomic DNA of the cells.

In some embodiments the method further comprises culturing an antigen reactive cell for a time period for clonal expansion of the cell.

In some embodiments, the disclosure provides a method for identifying antigen-specific cells comprising the method according to the sixth aspect, followed by sequencing the barcode in the clonally expanded cell.

In some embodiments, the disclosure provides a method for determining epitope specificity of B cells comprising the method according to the sixth aspect, followed by sequencing the barcode in the clonally expanded cell and comparing the sequence with known barcode relationships to epitope specificities.

In some embodiments, the disclosure provides a method for designing sequential immunization protocols comprising using differentially barcoded vaccines as sequential immunogens and determining by barcode sequencing which sequential immunizations produce broadly neutralizing antibodies (bNAbs).

In some embodiments, the disclosure provides a method for determining B call fate upon immunization comprising immunizing a mammal having an antigen-specific antibody repertoire with a mixture of differentially barcoded vaccines and determining by barcode sequencing which antigens induce preferential differentiation toward memory B cells and which antigens induce preferential differentiation toward plasma cells.

The disclosure provides a method for determining B cell recall upon sequential immunization comprising using barcode sequencing to identify their history of antigen encounters.

The disclosure also provides a method for identifying epitope dominance of an antigen comprising immunizing a mammal having an antigen-specific antibody repertoire with a mixture of differentially barcoded viral particles and determining by performing sequencing the barcodes of those cells which exhibit the greatest immune response to the antigen.

The disclosure provides a method for identifying and characterizing B cell clones based upon the sites of barcode integration by the method according to the sixth aspect, followed inverse PCR (iPCR) or splinkerette PCR.

To specifically target the barcoding system to antigen-specific B cells, and not generally to AAV infected cells, the VP1 protein of the AAV capsid is mutated at particular sites previously reported to prevent receptor-mediated transduction. In addition, VP1 can be engineered so that the AAV particle can be conjugated to different antigens of interest and allow antigen-specific recognition of the AAV through the BCR. VP1 is engineered to be expressed as a fusion protein with the SpyCatcher protein (116 amino acids). SpyCatcher, in the presence of a protein fused to a SpyTag peptide (13 amino acids), spontaneously reacts to form an intermolecular covalent isopeptide bond. The SpyCatcher/Spy Tag system has been widely used for irreversible conjugation of recombinant proteins. In fact, previous work showed that this system was highly efficient to conjugate Spytagged HIV-1 Env trimers to the surface of Virus Like Particles carrying the SpyCatcher subunit. The DNA sequence encoding for SpyCatcher is engineered at a specific site in the VP1 sequence, which has been previously reported to accommodate the sequence of a nanobody of 110-130 amino acids with no significant effect on AAV stability. This system can be used to conjugate any antigen of interest to the surface of the AAV particle.

In order to permanently barcode antigen specific B cells, the SB transposition system, previously used for an array of applications including transgenesis and insertional mutagenesis will be employed. The SB transposase enzyme recognizes specific DNA sites, induces the excision of the flanked DNA fragment and mediates its integration at a DNA target site. The AAV genome is engineered to encode for the SB100× transposase enzyme under the regulation of the Cytomegalovirus (CMV) promoter: CGTTACATAACTTACGGTAAATGGCCCGCCTGGCTGACCGCCCAACGACCCCCGCCC ATTGACGTCAATAATGACGTATGTTCCCATAGTAACGCCAATAGGGACTTTCCATTGAC GTCAATGGGTGGAGTATTTACGGTAAACTGCCCACTTGGCAGTACATCAAGTGTATCA TATGCCAAGTACGCCCCCTATTGACGTCAATGACGGTAAATGGCCCGCCTGGCATTAT GCCCAGTACATGACCTTATGGGACTTTCCTACTTGGCAGTACATCTACGTATTAGTCAT CGCTATTACCATGGTGATGCGGTTTTGGCAGTACATCAATGGGCGTGGATAGCGGTTT GACTCACGGGGATTTCCAAGTCTCCACCCCATTGACGTCAATGGGAGTTTGTTTTGGC ACCAAAATCAACGGGACTTTCCAAAATGTCGTAACAACTCCGCCCCATTGACGCAAA TGGGCGGTAGGCGTGTACGGTGGGAGGTCTATATAAGCAGAGCT [SEQ ID NO. 6], and a DNA barcode flanked by inverted terminal repeats (ITRs) containing the SB recognition sites. Upon BCR-mediated internalization of the antigen-pseudotyped AAV, the SB transposase is expressed and induces the integration of the DNA barcode in the genome of the B cell. This way, the B cell remains permanently labelled with a specific barcode, which is easily detected by regular PCR, q-PCR or NGS approaches. Since cells are expected to engulf multiple AAV particles, multiple copies of the barcode are integrated, further facilitating detection. Barcodes are permanently integrated in the B cell genome; therefore, they are inherited by the progeny upon proliferation. Different barcodes are used in combination with different pseudotyped AAVs to identify and classify B cells according to their reactivity. An additional advantage of this technology is that we can identify integration sites and use these sites as unique features to define and categorize B cell clones.

The AAV-SB system is used to barcode cells in some embodiments. To do this, infectious AAV2 particles carrying the SB transposase and the barcode system are used to transduce permissive Ramos B cells in vitro. Upon in vitro transduction, the cells are lysed and PCR, q-PCR and/or NGS is used to detect the presence of the barcode. This is done at different time points after transduction to confirm permanent barcoding. Previously reported methods can be used to identify the barcode integration sites, including inverse PCR (iPCR) or splinkerette PCR4, 5. Once the barcoding system is validated, non-infectious, Env-pseudotyped AAVs are produced and used to barcode antigen-specific mouse primary B cells. The pseudotyped AAVs are tested in cultures of B cells isolated from Ig KI mice carrying anti-Env antibodies2, and in polyclonal B cells isolated from wild type mice. To test the barcoding system in vivo, the Env-pseudotyped AAVs are used to immunize mice and eventually macaques. The elicited antibody and B cell responses are characterized and the presence of integrated barcodes in B cells are analyzed. Initially analyzed are the mouse responses to a single Env-AAV carrying one barcode, and then extended to analyze the responses to sequential immunization using series of different Env-AAVs carrying different barcodes. The AAV technology is used with the aptamer technology to produce Env-aptamer-AAVs and evaluate them in macaques. This first-of-its-kind technology opens a myriad of exciting avenues of investigation related to vaccine design and the B cell and antibody responses to vaccination.

The following Examples are intended to further illustrate certain embodiments of the disclosure and are not to be construed as limiting the scope of the disclosure in any way.

### Example 1

### Evaluation of the Suitability of Aptamers for Epitope Masking

To investigate the suitability of aptamers to identify antigen-specific B cells, a random 100-mer ssDNA oligo which is biotinylated at the 5′ end and fluoresceinated at the 3′ end was used. This oligo was incubated with SAV conjugated to the 4-hydroxy-3-nitrophenyl acetyl (NP) hapten, and to the fluorophore Alexa Fluor 647 (SAV-AF647-NP) (FIG. 3A). SAV-AF647-NP-oligo, and different variants lacking the NP or oligo components were used as controls, to stimulate transgenic B cells carrying an anti-NP specific antibody (B1-8hi Lambda antibody) in vitro. Using this system, B cells acquiring the oligo through NP-BCR interaction and internalization, are labelled with the AF647 and fluorescein fluorophores. In addition, lower surface expression of the lambda BCR further indicates antigen-BCR interaction and internalization. In fact, the analysis of the stimulated B cells by flow cytometry showed that B cells stimulated with the SAV-AF647-NP-oligo, but not those stimulated with SAV-AF647, SAV-AF647-NP or SAV-AF647-oligo, internalized the fluoresceinated oligo, indicating that the internalization was dependent on specific NP-BCR interaction (FIG. 3B).

To investigate whether aptamers are internalized in complex with their specific antigens, and whether the aptamers can be detected inside the cells, complexes of a His-tagged HIV-1 Env protein and a fluoresceinated anti-His-tag aptamer (FIG. 3C) were prepared. These complexes were used to stimulate B cells expressing an anti-Env antibody¹³ in vitro. Flow cytometry analysis has shown that only the B cells incubated with the Env-aptamer complexes, but not those incubated with the individual components, internalize the aptamer as evidenced by fluorescein detection and lower surface expression of BCR(FIG. 3D).

Altogether, these results indicate that aptamers: 1) bind and mask epitopes; 2) are internalized with antigen upon antigen-BCR interaction; and 3) can be detected inside the cell. In summary, the technology based on the use of aptamers shows great promise to modulate and characterize antibody responses to vaccination, which will importantly contribute to guide vaccine design efforts aiming to induce bNAbs.

### Example 2

### Production of AAV-SB System Encoding SB100× Transposase and Barcode

In order to track antigen-specific B cells in response to our model of sequential immunization, we will develop a new technology based on the use of pseudotyped Adeno Associated Viruses (AAVs) and the Sleeping Beauty (SB) system of gene transposition³⁶. We will combine this technology with the use of aptamers to mask immunodominant epitopes of antigens, which will result in a remarkable immunogen to guide and characterize the process of bNAb development upon vaccination. We will develop a pioneer technology to permanently barcode antigen-specific B cells in vivo. This approach will allow to track antigen-B-cell interactions upon vaccination as long as the B cell is alive. We will use this system to analyze the B cell response to sequential immunization. In particular, we will investigate the biological processes of immunodominance, antibody maturation, B cell fate and memory B cell recall upon sequential immunization. To do this, we will design a new immunogen system based on an AAV particle, which will expose the antigen of interest, optionally covered with specific barcodes on its surface or carrying barcodes within its genome. The AAV genome will be engineered to encode for the SB100× transposase, which will mediate the transposition of a DNA barcode provided in cis into the genome of the antigen-specific B cell (FIGS. 7B and 8).

In order to design a system to permanently barcode antigen-specific B cells, we will take advantage of AAVs and their ability to carry their genomes to the cell nucleus. AAVs are ssDNA viruses which have been widely used for gene therapy approaches. AAVs can infect a broad diversity of cell types in a receptor-mediated fashion.

To specifically target our barcoding system to antigen-specific B cells, and not generally to AAV infected cells, we will mutate the VP1 protein of the AAV capsid at particular sites previously reported to prevent receptor-mediated transduction38. In addition, we will engineer VP1 so that the AAV particle can be conjugated to different antigens of interest, and allow antigen-specific recognition of the AAV through the BCR. We will engineer VP1 to be expressed as a fusion protein with the SpyCatcher protein (116 amino acids) (FIG. 7). SpyCatcher, in the presence of a protein fused to a SpyTag peptide (13 amino acids), spontaneously reacts to form an intermolecular covalent isopeptide bond. The SpyCatcher/Spy Tag system has been widely used for irreversible conjugation of recombinant proteins. In fact, our previous work showed that this system was highly efficient to conjugate Spytagged HIV-1 Env trimers to the surface of Virus Like Particles carrying the SpyCatcher subunit12, 40. We will insert the DNA sequence encoding for SpyCatcher at a specific site in the VP1 sequence, which has been previously reported to accommodate the sequence of a nanobody of 110-130 amino acids with no significant effect on AAV stability. This system can be used to conjugate any antigen of interest to the surface of the AAV particle; we will evaluate our approach using already available Spytagged HIV-1 Env proteins that we will produce at The Wistar Institute, and use the Env-pseudotyped AAVs to investigate Env-specific B cell responses as explained below.

A schematic of the AAV-SB construct encoding the SB transposase and a DNA barcode is shown in FIG. 4. In order to evaluate the barcoding system, AAV-SB particles were produced with a regular, non-Env pseudotyped AAV2 capsid. To produce AAV-SB particles, AAV cells were co-transfected with a plasmid containing the engineered AAV genome and two additional plasmids encoding for the AAV capsid and helper proteins. The AAV titers obtained with this new construct were comparable to other previous AAV productions. The expression of the SB100× transposase was confirmed in the transfected 293AAV cells by western blot, indicating that the new construct is functional (FIG. 5). Infectious AAV2 particles carrying the SB transposase and the barcode system, are used to transduce permissive Ramos B cells in vitro. Upon in vitro transduction, cells are lysed and PCR, q-PCR and/or NGS is used to detect the presence of the barcode. This is done at different time points after transduction to confirm permanent barcoding. To identify the barcode integration sites, inverse PCR (iPCR) or splinkerette PCR is used. To test the barcoding system in vivo, the Env-pseudotyped AAVs is utilized to immunize mice. The elicited antibody and B cell responses are characterized, and the presence of integrated barcodes in B cells is analyzed.

### Example 3

### Tracking B-Cell Responses to Guide Vaccine Design

In this proposal, we propose a novel technology to identify and characterize antigen-specific B cells, record their history of B-cell-antigen encounters, and track the development of broadly neutralizing antibodies (bNAbs) against HIV-1 upon vaccination in vivo.

Previous studies have shown that a novel vaccination strategy involving sequential immunization was required to elicit bNAbs against HIV-1. This immunization approach required prime immunization with an engineered HIV-1 envelope (Env) protein followed by a series of boost immunizations with gradually more native-looking Envs. Sequential immunization was necessary to initiate and sustain a germinal center reaction where B cell receptors (BCRs) could gradually mature towards bNAb development through multiple rounds of affinity maturation. These studies were performed using a pioneering immunoglobulin knock-in (Ig KI) mouse where all B cells expressed the same antibody, the precursor of a human bNAb. Remarkably, this precursor became a highly mutated and potent bNAb upon sequential immunization. However, this Ig KI mouse model significantly simplified the B cell responses to immunization, since it circumvented the effects of epitope immunodominance and the competition by polyclonal B cells in the germinal centers. Unfortunately, no sequential immunization protocol has been able to induce protective levels of bNAbs in the context of a wild type B-cell repertoire, where multiple different competing B cell clonotypes respond to immunization.

We hypothesize that tracking the evolution of the B cell and antibody responses upon sequential immunization will guide the design of efficacious vaccines that can shepherd the development of bNAbs against highly mutating pathogens.

We will develop a groundbreaking technology to track antigen-specific B cells in response to our model of sequential immunization. Our innovative system will be based on the use of pseudotyped Adeno Associated Viruses (AAVs) and the Sleeping Beauty (SB) system of gene transposition and will allow to permanently barcode antigen-specific B cells in vivo (FIG. 4 and FIG. 5).

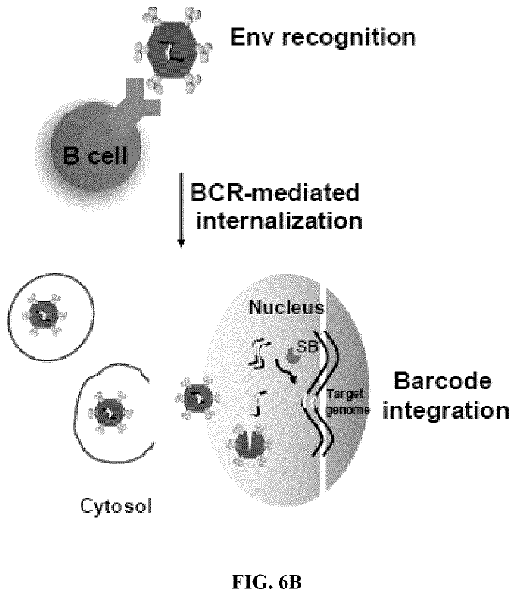

We will track antigen-specific B cells in response to our model of sequential immunization. Our innovative system will be based on the use of pseudotyped Adeno Associated Viruses (AAVs) and the Sleeping Beauty (SB) system of gene transposition and will allow to permanently barcode antigen-specific B cells in vivo (FIGS. 6A and 6B). We will use our new technology to investigate the biological processes of epitope immunodominance, antibody maturation, B cell fate and memory B cell recall upon sequential immunization.

We propose the following specific aims:
Designing a new system based on AAVs and the SB transposase to barcode antigen-specific B cells upon immunization.
Evaluating the use of the new AAV-SB system to identify, track and characterize antigen-specific B cells upon sequential immunization.

This system involves an antigen pseudotyped and non-infectious AAV particle which genome encodes a transposase enzyme and a transposon of known sequence (barcode). Upon specific recognition of the antigen on the AAV surface, a B cell will internalize the AAV particle, the transposase will be expressed and will induce the random integration of the barcode in the genome of the cell (FIG. 6B). This barcode and its integration site can be subsequently identified. The identification of a barcode in a B cell will indicate that this B cell specifically recognized the antigen on the AAV surface. The barcode integration sites in different B cells will allow us to establish clonal relationships between cells, since cells with identical integration sites will only be possible through proliferation of a founder cell.

The first goal during this reporting period was to generate and validate an antigen pseudotyped and non-infectious AAV particle.

First, we have engineered a new AAV particle which capsid can be conjugated to different antigens. To do this, we took advantage of the SpyCatcher-Spytag conjugation system. The SpyCatcher protein and the Spytag peptide establish spontaneous covalent bonds. Thus, we engineered the AAV capsid to express SpyCatcher on its surface. In this way, we could conjugate any Spytagged antigen to the AAV particle (FIGS. 6A and 6B). The AAV capsid is formed by 3 different capsid proteins, VP1, VP2 and VP3 which are splicing variants. To express SpyCatcher on the AAV capsid, we engineered VP1 to be expressed as a fusion protein with SpyCatcher while VP2 and VP3 remained wild type (FIG. 7). We have produced AAV particles expressing the VP1-SpyCatcher fusion protein. We have confirmed that the AAV particles tolerate well this modification. We have not observed a significant impact on the titers of our AAV productions when comparing wild type and SpyCatcher-AAVs. Moreover, we have confirmed that SpyCatcher is expressed on the AAV surface through an ELISA assay using an anti-SpyCatcher antibody (FIG. 8A). We have also confirmed that Spytagged proteins can be conjugated to the surface of the SpyCatcher-AAV. To show this, we have done ELISA assays and negative staining Electron Microscopy (nsEM) using our engineered AAVs. Our ELISA results show that Spytagged HIV-1 Envelope proteins can be conjugated to the surface of our SpyCatcher-AAVs. (FIG. 8B). Our nsEM images show that a Spytagged HIV-1 Envelope protein is efficiently conjugated to our engineered SpyCatcher-AAVs (FIG. 8C).

Second, we have engineered the AAV capsid to abolish the capacity of the AAV to interact with heparan sulfate, thus its capacity to infect cells (FIG. 8C and FIG. 7). This is necessary to ensure that barcoding will only occur in B cells that specifically recognize the antigen conjugated to the AAV surface and to prevent barcoding cells through infection. To achieve this, we mutated residues of the VP1, VP2 and VP3 capsid proteins that were previously reported to mediate AAV interaction with heparan sulfate. We have produced AAV particles carrying mutated VP1, VP2 and VP3 capsid proteins. We have confirmed that AAV particles using this engineered capsid assemble well. We have also confirmed that these mutations render the AAV unable to transduce cells in culture (data not shown).

Another main goal during this reporting period has been to evaluate and validate the use of the SB transposase enzyme to induce the random and permanent integration of a barcode in the genome of a cell.

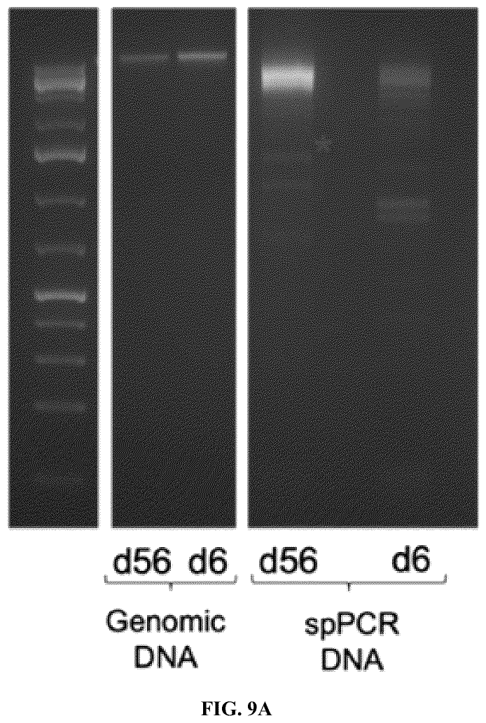

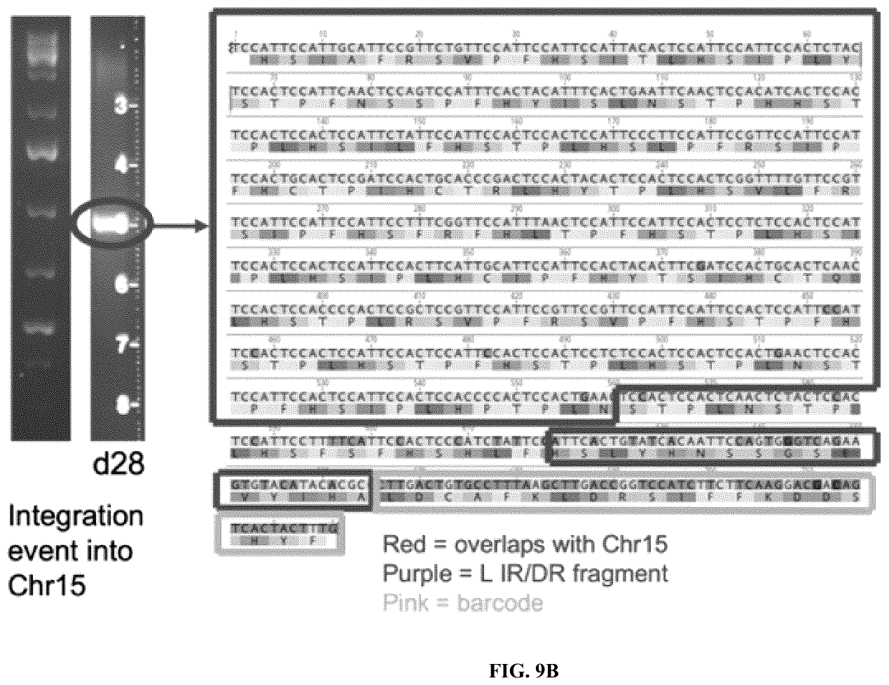

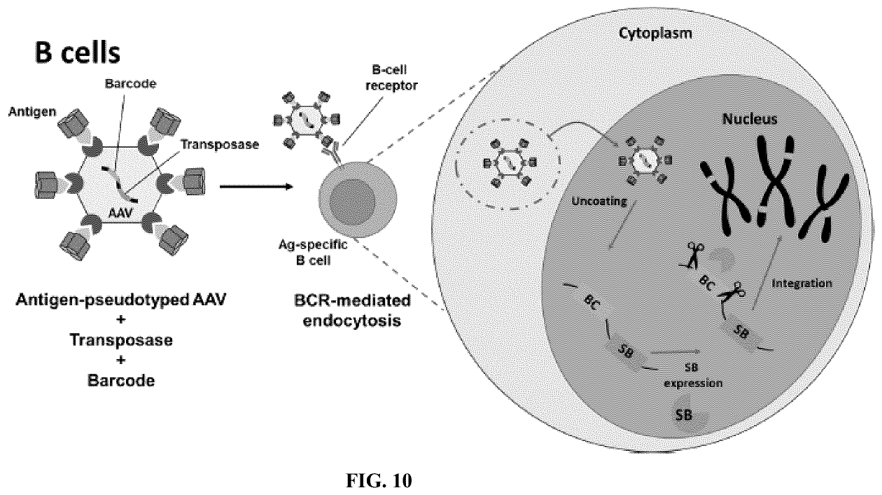

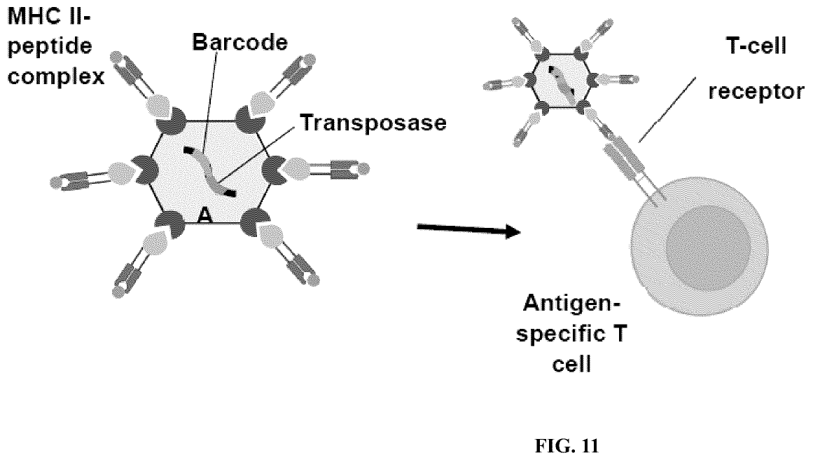

To investigate whether our SB-AAV system was able to induce the integration of barcodes in the genome of cells, we used a sophisticated PCR technique called Splinkerette PCR. We incubated HEK293 cells with our engineered SB-AAVs and obtained bulk genomic DNA from these cells at different time points after AAV transduction. We then used this genomic DNA for Splinkerette PCR. This method involved the digestion of genomic DNA with specific restriction enzymes and the ligation of Splinkerette oligo adapters of known sequence to one of the edges of the resulting genomic DNA fragments. Then, using primers annealing to the splinkerette adapters and the barcode, we were able to amplify fragments of genomic DNA containing integrated barcodes (FIG. 9A). Our preliminary data showed that our SB-AAV system is able to induce the integration of barcodes in the genome of cells and that we can detect the presence of barcodes for more than 50 days (FIG. 9B). To investigate whether we could identify barcode integration sites, we purified the DNA bands amplified through Splinkerette PCR in the experiments above and sequenced them. Our preliminary results showed that we can identify the genomic regions flanking integrated barcodes, thus identify integration sites (FIG. 9B).

The experiments detailed above were performed using bulk cells, however, our system will need to work at the single cell level. To investigate whether we can identify barcodes and integration sites in single cells exposed to our engineered AAVs, we have designed a new reporter SB-AAV that will allow us to identify cells that acquired AAV particles through the expression of the fluorescent protein mCherry. Since the efficiency of cell transduction or AAV acquisition is not 100% in cell cultures, the use of our new mCherry SB-AAV will allow us to specifically sort cells that internalized the AAV and study cell populations with potential to have integrated barcodes. To evaluate our new mCherry SB-AAV, we incubated this AAV with Ramos B cells engineered to express an anti-HIV antibody. Our preliminary data show that we can identify mCherry positive cells by flow cytometry (data not shown). We are currently sorting single mCherry cells and using them to investigate the presence of barcodes in the genomes of single cells. In this regard, we have optimized a method to purify and amplify genomic DNA from single cells. Using this method, we have been successful at amplifying different amplicons from single cell genomic DNA. We are currently using this method to amplify integrated barcodes by splinkerette PCR.

### References, Each of which is Incorporated by Reference in its Entirety

1 Escolano, A. et al. Sequential Immunization Elicits Broadly Neutralizing Anti-HIV-1 Antibodies in Ig Knockin Mice. Cell 166, 1445-1458 e1412, doi: 10.1016/j.cell.2016.07.030 (2016).
2 Steichen, J. M. et al. HIV Vaccine Design to Target Germline Precursors of Glycan-Dependent Broadly Neutralizing Antibodies. Immunity 45, 483-496, doi: 10.1016/j.immuni.2016.08.016 (2016).
3 Victora, G. D. & Nussenzweig, M. C. Germinal centers. Annu Rev Immunol 30, 429-457, doi: 10.1146/annurev-immunol-020711-075032 (2012).
4 Mates, L. et al. Molecular evolution of a novel hyperactive Sleeping Beauty transposase enables robust stable gene transfer in vertebrates. Nat Genet 41, 753-761, doi: 10.1038/ng.343 (2009).
5 Zakeri, B. et al. Peptide tag forming a rapid covalent bond to a protein, through engineering a bacterial adhesin. Proc Natl Acad Sci USA 109, E690-697, doi: 10.1073/pnas.1115485109 (2012).
6 Wang, D., Tai, P. W. L. & Gao, G. Adeno-associated virus vector as a platform for gene therapy delivery. Nat Rev Drug Discov 18, 358-378, doi: 10.1038/s41573-019-0012-9 (2019).
7 Uren, A. G. et al. A high-throughput splinkerette-PCR method for the isolation and sequencing of retroviral insertion sites. Nat Protoc 4, 789-798, doi: 10.1038/nprot.2009.64 (2009).

1. A nucleic acid molecule, wherein the nucleic acid molecule comprises:
(i) a barcode domain;
(ii) a nucleic acid sequence encoding a gene editing enzyme or functional variant thereof; wherein the nucleic acid sequence encoding the gene editing enzyme or functional variant thereof is operably linked to a regulatory sequence; and
(iii) a first gene editing enzyme cleavage sequence and a second gene editing enzyme cleavage sequence; wherein the first gene editing enzyme cleavage sequence is positioned within about 20 nucleotides upstream from the 5′ end of the nucleic acid sequence encoding a gene editing enzyme or functional variant thereof, and wherein the second gene editing enzyme cleavage sequence is positioned within about 20 nucleotides downstream from the 3′ end of the nucleic acid sequence encoding a gene editing enzyme or functional variant thereof.

2. The nucleic acid molecule of claim 1, wherein the nucleic acid sequence encoding a gene editing enzyme or functional variant thereof encodes a transposase, meganuclease, or Cas protein; and wherein the first gene editing enzyme cleavage sequence and a second gene editing enzyme cleavage sequence is a transposase, meganuclease, or Cas protein recognition and cleavage site, respectively.

3. The nucleic acid molecule of claim 1, wherein, in 5′ to 3′ orientation, the nucleic acid molecule comprises:
(i) a nucleic acid sequence encoding a gene editing enzyme or functional variant thereof;
(ii) a first gene editing enzyme cleavage sequence;
(iii) a barcode sequence;
(iv) a second gene editing enzyme cleavage sequence

4. The nucleic acid molecule of claim 1, wherein the regulatory sequence is positioned with about 50 nucleotides upstream from the 5′ end of the nucleic acid sequence encoding a gene editing enzyme or functional variant thereof.

5. A composition comprising a viral vector comprising the nucleic acid molecule of claim 1.

6. The composition of claim 5, wherein the viral vector is an AAV vector.

7. The composition of claim 5, wherein the viral vector comprises a capsid protein comprising a targeting domain.

8. The composition of claim 5, wherein the targeting domain associates with an amino acid sequence on a B cell, a T cell or a NK cell.

9. The composition of claim 5, wherein the targeting domain comprises a viral ENV protein or a functional variant thereof.

10. The composition of claim 5, wherein the targeting domain comprises gp120 or a functional variant thereof.

11. The composition of claim 5, wherein the targeting domain comprises a SpyCatcher domain.

12. The composition of claim 5, wherein the viral vector is an AAV vector that comprises a capsid comprising VP1, VP2 and VP3 amino acids; and wherein the amino acid sequence of at least one VP protein comprises a mutation within a VP2/3 splice acceptor site.

13. The composition claim 5 further comprising a cell.

14. A cell comprising the nucleic acid molecule of claim 1.

15. The composition of claim 14, wherein the cell is a B cell.

16. A method of identifying immune cell reactivity to an epitope comprising
(a) exposing an immune cell to the composition of claim 5 for a time period sufficient for the barcode region to integrate within genomic DNA of the immune cell;
(b) exposing an antigen comprising an epitope to the immune cell for a time period sufficient to elicit an epitope-specific immune response;
(c) identifying the immune cell reactivity to the epitope by correlating the epitope-specific immune response to the presence of the barcode region in the genomic DNA of the immune cell.

17. The method of claim 16 wherein the step of identifying the immune cell reactivity comprises identifying the sequence of the barcode domain by sequencing the barcode domain or detecting a probe associated with the barcode domain in the genomic DNA.

18. The method of claim 16, wherein the immune cell is a T cell or a B cell.

19. The method of claim 16, wherein the epitope-specific immune response is the activation of a B cell by stimulation of expression of an antibody or antibody fragment that associates with the epitope.

20. The method of claim 16 further comprising a step of sequencing the antibody or antibody fragment from the immune cell by PCR or a sequencing reaction and identifying the immune cell reactivity by correlating the antibody or antibody fragment sequence to detection of or sequence of the barcode domain.

21. The method of claim 16, wherein the immune cell expresses Spytag and the viral vector expresses SpyCatcher, or respective functional variants thereof.

22. The method of claim 16 further comprising a step of isolating the immune cell by cell sorting after step (b).


SEQUENCE LISTING (27 sequences)

SEQ ID NO: 1 - DNA, 110 bp, Adeno-associated virus
  source 1..110: mol_type = genomic DNA
tcgactctgacggttcactaaacgagctctgcttatatagcaacctgagtgatggctccgcccacgcgtgcacgtcaccgttaccggagcaacctgacaccggctccagc

SEQ ID NO: 2 - protein, 229 aa, synthetic construct
PSGHSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMDAVQYVDILKQHLKTSVRKLKLGRKWVFQHDNDPKHTSKVVAKWLKDNKVKVLEWPSQSPDLNPIENLWAELKKRVRARRPTNLTQLHQLCQEEWAKIHPNYCGKLVEGYPKRLTQVKQFKGNATKY

SEQ ID NO: 3 - protein, 339 aa, synthetic construct
MGKSKEISQDLRKRIVDLHKSGSSLGAISKRLAVPRSSVQTIVRKYKHHGTTQPSYRSGRRRYLSPRDERTLVRKVQINPRTTAKDLVKMLEETGTKVSISTVKRVLYRHNLKGHSARKKPLLQNRHKKARLRFATAHGDKDRTFWRNVLWSDETKIELFGHNDHRYVWRKKGEACKPKTIPTVKHGGGSIMLWGCFAAGGTGALHKIDGIMDAVQYVDILKQHLKTSVRKLKLGRKWVFQHDNDPKHTSKVVAKWLKDNKVKVLEWPSQSPDLNPIENLWAELKKRVRARRPTNLTQLHQLCQEEWAKIHPNYCGKLVEGYPKRLTQVKQFKGNATKY

SEQ ID NO: 4 - DNA, 420 bp, synthetic construct
  source 1..420: mol_type = other DNA
atgtcgtactaccatcaccatcaccatcacgattacgacatcccaacgaccgaaaacctgtattttcagggcgccatggttgataccttatcaggtttatcaagtgagcaaggtcagtccggtgatatgacaattgaagaagatagtgctacccatattaaattctcaaaacgtgatgaggacggcaaagagttagctggtgcaactatggagttgcgtgattcatctggtaaaactattagtacatggatttcagatggacaagtgaaagatttctacctgtatccaggaaaatatacatttgtcgaaaccgcagcaccagacggttatgaggtagcaactgctattacctttacagttaatgagcaaggtcaggttactgtaaatggcaaagcaactaaaggtgacgctcatatttaa

SEQ ID NO: 5 - protein, 13 aa, synthetic construct
AHIVMVDAYKPTK

SEQ ID NO: 6 - DNA, 508 bp, synthetic construct
  source 1..508: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 7 - protein, 1091 aa, synthetic construct
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQGQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPGEWLVWKQSCSSTSSTHSAAASLQCPSQVLPPASPEGETVADLQTQ

SEQ ID NO: 8 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQAGNAQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 9 - DNA, 234 bp, synthetic construct
  source 1..234: mol_type = other DNA
agcttgaccggtacagtacgctcagttctatctgacctatatgagacgtcaactttttcaagagtgccacgcccgaaggtcacgtatagggcagaactatatctgtcaaagatgacggtaccgacacgatatttgctgaagtccattccgaaggtgatgtacgtcgattcttatggaattaatctcgtcgcgcgatgttgtgccacaacgtctatatgatggcgtacgtaaagg

SEQ ID NO: 10 - DNA, 234 bp, synthetic construct
  source 1..234: mol_type = other DNA
agcttgaccggtacagtaagagcaccagtgccttgattctactcagtctcaactttttcaagagtgccacgcccgaaggtcacgtatagggcagaactatatctgtcaaagatgacggtaccgacacgatatttgctgaagtccattccgaaggtgatgtacgtcgatttcgtctgactgaacatacggcctatgactcgtgccacaacgtctatatgatggcgtacgtaaagg

SEQ ID NO: 11 - DNA, 237 bp, synthetic construct
  source 1..237: mol_type = other DNA
agcttgaccggtacagtataatggcaaggtgccgcttccggcaatggaacaactttttcaagagtgccacgcccgaaggtcacgtatagggcagaactatatctgtcactagagatgacggtaccgacacgatatttgctgaagtccattccgaaggtgatgtacgtcgattgccgtaaccgaaccattctcggttgcctctgtgccacaacgtctatatgatggcgtacgtaaagg

SEQ ID NO: 12 - DNA, 234 bp, synthetic construct
  source 1..234: mol_type = other DNA
agcttgaccggtacagtactaatgatggtcggcctatcagtcaaccattcaactttttcaagagtgccacgcccgaaggtcacgtatagggcagaactatatctgtcaaagatgacggtaccgacacgatatttgctgaagtccattccgaaggtgatgtacgtcgattgagcgcaataaacaaggcgtgtatgtagaagtgccacaacgtctatatgatggcgtacgtaaagg

SEQ ID NO: 13 - DNA, 234 bp, synthetic construct
  source 1..234: mol_type = other DNA
agcttgaccggtacagtattctatggttcctcgcaacctggatgcttatcaactttttcaagagtgccacgcccgaaggtcacgtatagggcagaactatatctgtcaaagatgacggtaccgacacgatatttgctgaagtccattccgaaggtgatgtacgtcgattatgtcgtggtagagtgcggctgcctggtgggtgccacaacgtctatatgatggcgtacgtaaagg

SEQ ID NO: 14 - protein, 596 aa, synthetic construct
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTPJVICRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKLLQEPYKLTIVGTVRSNKREIPEVLKSRSRPVGTSMFCFDGPLTLVSYKPKPAIGVIVYLLSSCDEDASINESTGKPQMVMYYNQTKGGVDTLDQMCSVMTCSRKTORWPMALLYGMINIACINSFIIYSHNVSSKGEKVQSPJKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 15 - protein, 340 aa, synthetic construct
MGKSKEISQDLRKKIVDLHKSGSSLGAISKRLKVPRSSVQTIVRKYKHHGTTQPSYRSGRRRYLSPRDERTLVRKVQINPRTTAKDLVKMLEETGTKVSISTVKRVLYRHNLKGRSARKKPLLQNPAKKARLRFATAHGDKDRTFWRNVLWSDETKIELFGHNDHRYVWRKKGEACKPKNTIPTVKHGGGSIMLWGCFAAGGTGALHKIDGIMRKENYVDILKQHLKTSVRKLKLGRKWVFQMDNDPKHTSKVVAKWLKDNKVKVLEWPSQSPDLNPIENLWAELKKRVRARRPTNLTQLHQLCQEEWAKIHPTYCGKLVEGYPKRLTQVKQFKGNATKY

SEQ ID NO: 16 - DNA, 30 bp, synthetic construct
  source 1..30: mol_type = other DNA
gttgaagtcggaagtttacatacacttaag

SEQ ID NO: 17 - DNA, 30 bp, synthetic construct
  source 1..30: mol_type = other DNA
cagtgggtcagaagtttacatacactaagg

SEQ ID NO: 18 - DNA, 31 bp, synthetic construct
  source 1..31: mol_type = other DNA
cagtgggtcagaagttaacatacactcaatt

SEQ ID NO: 19 - DNA, 31 bp, synthetic construct
  source 1..31: mol_type = other DNA
agttgaagtcggaagtttacatacaccttag

SEQ ID NO: 20 - DNA, 30 bp, synthetic construct
  source 1..30: mol_type = other DNA
caktgrgtcrgaagtttacatacacttaag

SEQ ID NO: 21 - DNA, 226 bp, synthetic construct
  source 1..226: mol_type = other DNA
agttgaagtcggaagtttacatacacttaagttggagtcattaaaactcgtttttcaactacaccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaa

SEQ ID NO: 22 - DNA, 228 bp, synthetic construct
  source 1..228: mol_type = other DNA
ttgagtgtatgttaacttctgacccactgggaatgtgatgaaagaaataaagctgaaatgaatcattctctctactattattctgatatttcacattcttaaaataaagtggtgatcctaactgaccttaagacagggaatctttactcggattaaatgtcaggaattgtgaaaaagtgagtttaaatgtatttggctaaggtgtatgtaaacttccgacttcaactg

SEQ ID NO: 23 - DNA, 725 bp, synthetic construct
  source 1..725: mol_type = other DNA
tccattccattgcattccgttctgttccattccattccattacactccattccattccactctactccactccattcaactccagtccatttcactacatttcactgaattcaactccacatcactccactccactccactccattctattccattccactccactccattcccttccattccgttccattccattccactgcactccgatccactgcacccgactccactacactccactccactcggttttgttccgttccattccattccattcctttcggttccatttaactccattccattccactcctctccactccattccactccactccattccacttcattgcattccattccactacacttcgatccactgcactcaactccactccaccccactccgctccgttccattccgttccgttccattccattccactccattccattccactccactccattccactccattccactccactcctctccactccactccactgaactccactccattccactccattccactccaccccactccactgaacccactccactcaactctactccactccattccttttcattccactcccatctattccattcactgtatcacaattccagtgggtcagaagtgtacatacacgcccttgactgtgcctttaagcttgaccggtccatcttcttcaaggacgacagtcactactttg

SEQ ID NO: 24 - protein, 242 aa, synthetic construct
HSIAFRSVPFHSITLHSIPLYSTPFFNSSPFHYISLNSTPHHSTPLHSILFHSTPLHSLPFRSIPFHCTPIHCTRLHYTPLHSVLFRSIPFHSFRFHLTPFHSTPLHSIPLHSIPLHCIPFHYTSIHCTQLHSTPLRSVPFRSVPFHSTPFHSTPLHSTPFHSTPLHSTPLNSTPFHSIPLHPTPLNSTPLNSTPLHSFSFHSHLFHSLYHNSSGSEVYIHALDCAFKLDRSIFFKDDSHFY

SEQ ID NO: 25 - protein, 1207 aa, synthetic construct
MLLTLIILLPVVSKFSFVSLSAPQHWSCPEGTLAGNGNSTCVGPAPFLIFSHGNSIFRIDTEGTNYEQLVVDAGVSVIMDFHYNEKRIYWVDLERQLLQRVFLNGSRQERVCNIEKNVSGMAINWINEEVIWSNQQEGIITVTDMKGNNSHILLSALKYPANVAVDPVERFIFWSSEVAGSLYRADLDGVGVKALLETSEKITAVSLDVLDKRLFWIQYNREGSNSLICSCDYDGGSVHISKHPTQHNLFAMSLFGDRIFYSTWKMKTIWIANKHTGKDMVRINLHSSFVPLGELKVVHPLAQPKAEDDTWEPEQKLCKLRKGNCSSTVCGQDLQSHLCMCAEGYALSRDRKYCEDVNECAFWNHGCTLGCKNTPGSYYCTCPVGFVLLPDGKRCHQLVSCPRNVSECSHDCVLTSEGPLCFCPEGSVLERDGKTCSGCSSPDNGGCSQLCVPLSPVSWECDCFPGYDLQLDEKSCAASGPQPFLLFANSQDIRHMHFDGTDYGTLLSQQMGMVYALDHDPVENKIYFAHTALKWIERANMDGSQRERLIEEGVDVPEGLAVDWIGRRFYWTDRGKSLIGRSDLNGKRSKIITKENISQPRGIAVHPMAKRLFWTDTGINPRIESSSLQGLGRLVIASSDLIWPSGITIDFLTDKLYWCDAKQSVIEMANLDGSKRRRLTQNDVGHPFAVAVFEDYVWFSDWAMPSVMRVNKRTGKDRVRLQGSMLKPSSLVVVHPLAKPGADPCLYQNGGCEHICKKRLGTAWCSCREGFMKASDGKTCLALDGHQLLAGGEVDLKNQVTPLDILSKTRVSEDNITESQHMLVAEIMVSDQDDCAPVGCSMYARCISEGEDATCQCLKGFAGDGKLCSDIDECEMGVPVCPPASSKCINTEGGYVCRCSEGYQGDGIHCLDIDECQLGEHSCGENASCTNTEGGYTCMCAGRLSEPGLICPDSTPPPHLREDDHHYSVRNSDSECPLSHDGYCLHDGVCMYIEALDKYACNCVVGYIGERCQYRDLKWWELRHAGHGQQQKVIVVAVCVVVLVMLLLLSLWGAHYYRTQKLLSKNPKNPYEESSRDVRSRRPADTEDGMSSCPQPWFVVIKEHQDLKNGGQPVAGEDGQAADGSMQPTSWRQEPQLCGMGTEQGCWIPVSSDKGSCPQVMERSFHMPSYGTQTLEGGVEKPHSLLSANPLWQQRALDPPHQMELTQ

SEQ ID NO: 26 - protein, 296 aa, Adeno-associated virus
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKVLTKCQEEVSHIPAVHPGSFRPKCDENGNYLPLQCYGSIGYCWCVFPNGTEVPNTRSRGHHNCSESLELEDPSSGLGVTKQDLGPVPM

SEQ ID NO: 27 - DNA, 52 bp, synthetic construct
  source 1..52: mol_type = other DNA
gagctcggtaccctatacagttgaagtcggaagtttacatacacttaagttg